SEQUENCE LISTING (196 sequences)

SEQ ID NO: 1 - DNA, 8 bp, Artificial Sequence
  Synthetic DNA  15-Amy 3
ccatgttg

SEQ ID NO: 2 - DNA, 8 bp, Artificial Sequence
  Synthetic DNA  15-Amy 6
acaccggc

SEQ ID NO: 3 - DNA, 8 bp, Artificial Sequence
  Synthetic DNA  15-Amy 11
gtatccta

SEQ ID NO: 4 - DNA, 8 bp, Artificial Sequence
  Synthetic DNA  15-Amy 13
ggatgagc

SEQ ID NO: 5 - DNA, 8 bp, Artificial Sequence
  Synthetic DNA  15-Amy_14
gagactag

SEQ ID NO: 6 - DNA, 8 bp, Artificial Sequence
  Synthetic DNA  15-Amy 15
ggcctcta

SEQ ID NO: 7 - DNA, 8 bp, Artificial Sequence
  Synthetic DNA  15-Amy 17
caatgata

SEQ ID NO: 8 - DNA, 8 bp, Artificial Sequence
  Synthetic DNA  15-Amy 21
cctatcca

SEQ ID NO: 9 - DNA, 8 bp, Artificial Sequence
  Synthetic DNA  15-Amy 22
ttgatata

SEQ ID NO: 10 - DNA, 8 bp, Artificial Sequence
  Synthetic DNA  15-Amy 23
agcgatat

SEQ ID NO: 11 - DNA, 8 bp, Artificial Sequence
  Synthetic DNA  15-Amy 26
cctacagt

SEQ ID NO: 12 - DNA, 8 bp, Artificial Sequence
  Synthetic DNA  15-Amy 27
atgacagt

SEQ ID NO: 13 - DNA, 8 bp, Artificial Sequence
  Synthetic DNA  15-Amy 30
agtgtaca

SEQ ID NO: 14 - DNA, 8 bp, Artificial Sequence
  Synthetic DNA  15-Amy 31
ctggcacg

SEQ ID NO: 15 - DNA, 8 bp, Artificial Sequence
  Synthetic DNA  15-Amy 37
cgcctaac

SEQ ID NO: 16 - DNA, 8 bp, Artificial Sequence
  Synthetic DNA  15-Amy 38
ctcgtcgt

SEQ ID NO: 17 - DNA, 8 bp, Artificial Sequence
  Synthetic DNA  15-Amy 40
tacagaca

SEQ ID NO: 18 - DNA, 8 bp, Artificial Sequence
  Synthetic DNA  15-Amy 41
cagtacca

SEQ ID NO: 19 - DNA, 8 bp, Artificial Sequence
  Synthetic DNA  15-Amy_45
aaggtatc

SEQ ID NO: 20 - DNA, 8 bp, Artificial Sequence
  Synthetic DNA  15-Amy 46
aattgaat

SEQ ID NO: 21 - DNA, 8 bp, Artificial Sequence
  Synthetic DNA  15-Amy_47
cgcaagag

SEQ ID NO: 22 - DNA, 8 bp, Artificial Sequence
  Synthetic DNA  15-Amy 48
ctcgataa

SEQ ID NO: 23 - DNA, 8 bp, Artificial Sequence
  Synthetic DNA  15-Amy 49
ttgttctc

SEQ ID NO: 24 - DNA, 8 bp, Artificial Sequence
  Synthetic DNA  15-Amy_50
tgacatct

SEQ ID NO: 25 - DNA, 8 bp, Artificial Sequence
  Synthetic DNA  15-Amy 52
ttctgttc

SEQ ID NO: 26 - DNA, 8 bp, Artificial Sequence
  Synthetic DNA  15-Amy 54
tcagcacc

SEQ ID NO: 27 - DNA, 8 bp, Artificial Sequence
  Synthetic DNA  15-Amy 56
gttatcac

SEQ ID NO: 28 - DNA, 8 bp, Artificial Sequence
  Synthetic DNA  15-Amy 57
acgtgtcc

SEQ ID NO: 29 - DNA, 8 bp, Artificial Sequence
  Synthetic DNA  15-Amy 60
tggctcct

SEQ ID NO: 30 - DNA, 8 bp, Artificial Sequence
  Synthetic DNA  15-Amy 61
atgcgaag

SEQ ID NO: 31 - DNA, 8 bp, Artificial Sequence
  Synthetic DNA  15-Amy 62
aactacct

SEQ ID NO: 32 - DNA, 8 bp, Artificial Sequence
  Synthetic DNA  15-Amy 65
tggtcata

SEQ ID NO: 33 - DNA, 8 bp, Artificial Sequence
  Synthetic DNA  15-Amy 68
acataaca

SEQ ID NO: 34 - DNA, 8 bp, Artificial Sequence
  Synthetic DNA  15-Amy_69
acaggcat

SEQ ID NO: 35 - DNA, 8 bp, Artificial Sequence
  Synthetic DNA  15-Amy 72
acctctct

SEQ ID NO: 36 - DNA, 8 bp, Artificial Sequence
  Synthetic DNA  15-Amy 88
agatgatt

SEQ ID NO: 37 - DNA, 8 bp, Artificial Sequence
  Synthetic DNA  15-Amy 92
agactctt

SEQ ID NO: 38 - DNA, 8 bp, Artificial Sequence
  Synthetic DNA  15-Amy_93
actagcag

SEQ ID NO: 39 - DNA, 8 bp, Artificial Sequence
  Synthetic DNA  15-Amy 95
atacacgt

SEQ ID NO: 40 - DNA, 8 bp, Artificial Sequence
  Synthetic DNA  15-Amy 96
acagcatt

SEQ ID NO: 41 - DNA, 8 bp, Artificial Sequence
  Synthetic DNA  15-Amy 102
ataattag

SEQ ID NO: 42 - DNA, 8 bp, Artificial Sequence
  Synthetic DNA  15-Amy 108
tctagacc

SEQ ID NO: 43 - DNA, 8 bp, Artificial Sequence
  Synthetic DNA  15-Amy 109
ctacagac

SEQ ID NO: 44 - DNA, 8 bp, Artificial Sequence
  Synthetic DNA  15-Amy 111
ctagttgc

SEQ ID NO: 45 - DNA, 8 bp, Artificial Sequence
  Synthetic DNA  15-Amy 115
cattgtac

SEQ ID NO: 46 - DNA, 8 bp, Artificial Sequence
  Synthetic DNA  15-Amy 116
agtatgat

SEQ ID NO: 47 - DNA, 8 bp, Artificial Sequence
  Synthetic DNA  15-Amy 117
agaatcaa

SEQ ID NO: 48 - DNA, 8 bp, Artificial Sequence
  Synthetic DNA  15-Amy 118
ttcattga

SEQ ID NO: 49 - DNA, 8 bp, Artificial Sequence
  Synthetic DNA  15-Amy 120
atcactta

SEQ ID NO: 50 - DNA, 8 bp, Artificial Sequence
  Synthetic DNA  15-Amy 121
tcaatcat

SEQ ID NO: 51 - DNA, 8 bp, Artificial Sequence
  Synthetic DNA  15-Amy 125
agatgtca

SEQ ID NO: 52 - DNA, 8 bp, Artificial Sequence
  Synthetic DNA  15-Amy 127
gtaatatg

SEQ ID NO: 53 - DNA, 8 bp, Artificial Sequence
  Synthetic DNA  15-Amy 129
ccacagca

SEQ ID NO: 54 - DNA, 8 bp, Artificial Sequence
  Synthetic DNA  15-Amy 131
catccacc

SEQ ID NO: 55 - DNA, 8 bp, Artificial Sequence
  Synthetic DNA  15-Amy_133
cagactca

SEQ ID NO: 56 - DNA, 8 bp, Artificial Sequence
  Synthetic DNA  15-Amy 135
acttcata

SEQ ID NO: 57 - DNA, 8 bp, Artificial Sequence
  Synthetic DNA  15-Amy 137
ccaacgga

SEQ ID NO: 58 - DNA, 8 bp, Artificial Sequence
  Synthetic DNA  15-Amy_141
accaatcc

SEQ ID NO: 59 - DNA, 8 bp, Artificial Sequence
  Synthetic DNA  15-Amy 145
tagcataa

SEQ ID NO: 60 - DNA, 8 bp, Artificial Sequence
  Synthetic DNA  15-Amy 146
tgacagga

SEQ ID NO: 61 - DNA, 8 bp, Artificial Sequence
  Synthetic DNA  15-Amy 147
catgaagt

SEQ ID NO: 62 - DNA, 8 bp, Artificial Sequence
  Synthetic DNA  15-Amy 150
tatagtag

SEQ ID NO: 63 - DNA, 8 bp, Artificial Sequence
  Synthetic DNA  15-Amy 152
accacatc

SEQ ID NO: 64 - DNA, 8 bp, Artificial Sequence
  Synthetic DNA  15-Amy 153
aattatag

SEQ ID NO: 65 - DNA, 8 bp, Artificial Sequence
  Synthetic DNA  15-Amy 156
ttccacat

SEQ ID NO: 66 - DNA, 8 bp, Artificial Sequence
  Synthetic DNA  15-Amy 158
caggcata

SEQ ID NO: 67 - DNA, 8 bp, Artificial Sequence
  Synthetic DNA  15-Amy 162
tagttaac

SEQ ID NO: 68 - DNA, 8 bp, Artificial Sequence
  Synthetic DNA  15-Amy 163
ccgcatct

SEQ ID NO: 69 - DNA, 8 bp, Artificial Sequence
  Synthetic DNA  15-Amy_164
atgaatct

SEQ ID NO: 70 - DNA, 8 bp, Artificial Sequence
  Synthetic DNA  15-Amy_168
tgtgactt

SEQ ID NO: 71 - DNA, 8 bp, Artificial Sequence
  Synthetic DNA  15-Amy 169
aggcttac

SEQ ID NO: 72 - DNA, 8 bp, Artificial Sequence
  Synthetic DNA  15-Amy 170
ctgtcctg

SEQ ID NO: 73 - DNA, 8 bp, Artificial Sequence
  Synthetic DNA  15-Amy_171
gatacatt

SEQ ID NO: 74 - DNA, 8 bp, Artificial Sequence
  Synthetic DNA  15-Amy 172
accggagt

SEQ ID NO: 75 - DNA, 8 bp, Artificial Sequence
  Synthetic DNA  15-Amy 173
tgaccttc

SEQ ID NO: 76 - DNA, 8 bp, Artificial Sequence
  Synthetic DNA  15-Amy 177
aggactaa

SEQ ID NO: 77 - DNA, 8 bp, Artificial Sequence
  Synthetic DNA  15-Amy 183
tcattgac

SEQ ID NO: 78 - DNA, 8 bp, Artificial Sequence
  Synthetic DNA  15-Amy 184
caggacat

SEQ ID NO: 79 - DNA, 8 bp, Artificial Sequence
  Synthetic DNA  15-Amy 185
taatactc

SEQ ID NO: 80 - DNA, 8 bp, Artificial Sequence
  Synthetic DNA  15-Amy 187
tatgcttc

SEQ ID NO: 81 - DNA, 8 bp, Artificial Sequence
  Synthetic DNA  15-Amy 195
ttaggaga

SEQ ID NO: 82 - DNA, 8 bp, Artificial Sequence
  Synthetic DNA  15-Amy 199
ggctaaga

SEQ ID NO: 83 - DNA, 8 bp, Artificial Sequence
  Synthetic DNA  15-Amy_201
tagtgagt

SEQ ID NO: 84 - DNA, 8 bp, Artificial Sequence
  Synthetic DNA  15-Amy 207
ccatcact

SEQ ID NO: 85 - DNA, 8 bp, Artificial Sequence
  Synthetic DNA  15-Amy 208
ttatagtt

SEQ ID NO: 86 - DNA, 8 bp, Artificial Sequence
  Synthetic DNA  15-Amy 210
agtacacc

SEQ ID NO: 87 - DNA, 8 bp, Artificial Sequence
  Synthetic DNA  15-Amy_211
cacttgag

SEQ ID NO: 88 - DNA, 8 bp, Artificial Sequence
  Synthetic DNA  15-Amy 213
agtccaag

SEQ ID NO: 89 - DNA, 8 bp, Artificial Sequence
  Synthetic DNA  15-Amy 215
tcactaca

SEQ ID NO: 90 - DNA, 8 bp, Artificial Sequence
  Synthetic DNA  15-Amy 216
agaattcc

SEQ ID NO: 91 - DNA, 8 bp, Artificial Sequence
  Synthetic DNA  15-Amy 218
aattaagc

SEQ ID NO: 92 - DNA, 8 bp, Artificial Sequence
  Synthetic DNA  15-Amy 219
acacctat

SEQ ID NO: 93 - DNA, 8 bp, Artificial Sequence
  Synthetic DNA  15-Amy 221
attgcaat

SEQ ID NO: 94 - DNA, 8 bp, Artificial Sequence
  Synthetic DNA  15-Amy 225
tggataat

SEQ ID NO: 95 - DNA, 8 bp, Artificial Sequence
  Synthetic DNA  I5-Amy  250
caatcgtc

SEQ ID NO: 96 - DNA, 8 bp, Artificial Sequence
  Synthetic DNA  I5-Amy  256
tagaagtc

SEQ ID NO: 97 - DNA, 8 bp, Artificial Sequence
  Synthetic DNA  17-Amy 1006
gctgtgtt

SEQ ID NO: 98 - DNA, 8 bp, Artificial Sequence
  Synthetic DNA  17-Amy 1017
atggcgac

SEQ ID NO: 99 - DNA, 8 bp, Artificial Sequence
  Synthetic DNA  17-Amy 1018
ggcgagca

SEQ ID NO: 100 - DNA, 8 bp, Artificial Sequence
  Synthetic DNA  17-Amy 1019
gctgtccg

SEQ ID NO: 101 - DNA, 8 bp, Artificial Sequence
  Synthetic DNA  17-Amy_1030
cgagtgaa

SEQ ID NO: 102 - DNA, 8 bp, Artificial Sequence
  Synthetic DNA  17-Amy 1033
ccatcact

SEQ ID NO: 103 - DNA, 8 bp, Artificial Sequence
  Synthetic DNA  17-Amy 1036
agtacacc

SEQ ID NO: 104 - DNA, 8 bp, Artificial Sequence
  Synthetic DNA  17-Amy 1049
tcgctgat

SEQ ID NO: 105 - DNA, 8 bp, Artificial Sequence
  Synthetic DNA  17-Amy 1052
ggcggtaa

SEQ ID NO: 106 - DNA, 8 bp, Artificial Sequence
  Synthetic DNA  17-Amy 1056
ccgccgaa

SEQ ID NO: 107 - DNA, 8 bp, Artificial Sequence
  Synthetic DNA  17-Amy 1057
gattgcga

SEQ ID NO: 108 - DNA, 8 bp, Artificial Sequence
  Synthetic DNA  17-Amy 1058
acattctc

SEQ ID NO: 109 - DNA, 8 bp, Artificial Sequence
  Synthetic DNA  17-Amy 1065
ccactggt

SEQ ID NO: 110 - DNA, 8 bp, Artificial Sequence
  Synthetic DNA  17-Amy 1078
cctgccaa

SEQ ID NO: 111 - DNA, 8 bp, Artificial Sequence
  Synthetic DNA  17-Amy 1080
atacgtcc

SEQ ID NO: 112 - DNA, 8 bp, Artificial Sequence
  Synthetic DNA  17-Amy 1091
tcaactct

SEQ ID NO: 113 - DNA, 8 bp, Artificial Sequence
  Synthetic DNA  17-Amy 1095
accgctac

SEQ ID NO: 114 - DNA, 8 bp, Artificial Sequence
  Synthetic DNA  17-Amy 1097
gcaatgct

SEQ ID NO: 115 - DNA, 8 bp, Artificial Sequence
  Synthetic DNA  17-Amy_1100
ggaccgcg

SEQ ID NO: 116 - DNA, 8 bp, Artificial Sequence
  Synthetic DNA  17-Amy 1101
cctactta

SEQ ID NO: 117 - DNA, 8 bp, Artificial Sequence
  Synthetic DNA  17-Amy_1102
gatgatct

SEQ ID NO: 118 - DNA, 8 bp, Artificial Sequence
  Synthetic DNA  17-Amy 1112
cagtggaa

SEQ ID NO: 119 - DNA, 8 bp, Artificial Sequence
  Synthetic DNA  17-Amy 1115
gttgacat

SEQ ID NO: 120 - DNA, 8 bp, Artificial Sequence
  Synthetic DNA  17-Amy_1125
gccataga

SEQ ID NO: 121 - DNA, 8 bp, Artificial Sequence
  Synthetic DNA  17-Amy 1134
tctggaat

SEQ ID NO: 122 - DNA, 8 bp, Artificial Sequence
  Synthetic DNA  17-Amy 1160
tgccgatc

SEQ ID NO: 123 - DNA, 8 bp, Artificial Sequence
  Synthetic DNA  17-Amy 1173
atgtagca

SEQ ID NO: 124 - DNA, 8 bp, Artificial Sequence
  Synthetic DNA  17-Amy 1174
gtcaccaa

SEQ ID NO: 125 - DNA, 8 bp, Artificial Sequence
  Synthetic DNA  17-Amy 1193
ctaagagt

SEQ ID NO: 126 - DNA, 8 bp, Artificial Sequence
  Synthetic DNA  17-Amy 1195
cctctctc

SEQ ID NO: 127 - DNA, 8 bp, Artificial Sequence
  Synthetic DNA  17-Amy 1199
gctaatga

SEQ ID NO: 128 - DNA, 8 bp, Artificial Sequence
  Synthetic DNA  17-Amy 1203
ctggtgat

SEQ ID NO: 129 - DNA, 8 bp, Artificial Sequence
  Synthetic DNA  17-Amy 1209
ctgatagc

SEQ ID NO: 130 - DNA, 8 bp, Artificial Sequence
  Synthetic DNA  17-Amy_1214
aatgccgg

SEQ ID NO: 131 - DNA, 8 bp, Artificial Sequence
  Synthetic DNA  17-Amy 1230
gcacattg

SEQ ID NO: 132 - DNA, 8 bp, Artificial Sequence
  Synthetic DNA  17-Amy 1233
tgttgcac

SEQ ID NO: 133 - DNA, 8 bp, Artificial Sequence
  Synthetic DNA  17-Amy 1234
gcctatcg

SEQ ID NO: 134 - DNA, 8 bp, Artificial Sequence
  Synthetic DNA  17-Amy_1244
gccttcgg

SEQ ID NO: 135 - DNA, 8 bp, Artificial Sequence
  Synthetic DNA  17-Amy 1249
tcggtgtc

SEQ ID NO: 136 - DNA, 8 bp, Artificial Sequence
  Synthetic DNA  17-Amy 1258
gcgacgta

SEQ ID NO: 137 - DNA, 8 bp, Artificial Sequence
  Synthetic DNA  17-Amy 1269
gcacgatt

SEQ ID NO: 138 - DNA, 8 bp, Artificial Sequence
  Synthetic DNA  17-Amy 1272
ctgctact

SEQ ID NO: 139 - DNA, 8 bp, Artificial Sequence
  Synthetic DNA  17-Amy 1274
gcttacaa

SEQ ID NO: 140 - DNA, 8 bp, Artificial Sequence
  Synthetic DNA  17-Amy 1276
tgaacaac

SEQ ID NO: 141 - DNA, 8 bp, Artificial Sequence
  Synthetic DNA  17-Amy 1281
acttgtaa

SEQ ID NO: 142 - DNA, 8 bp, Artificial Sequence
  Synthetic DNA  17-Amy 1310
tctgcgac

SEQ ID NO: 143 - DNA, 8 bp, Artificial Sequence
  Synthetic DNA  17-Amy 1311
ggccgagt

SEQ ID NO: 144 - DNA, 8 bp, Artificial Sequence
  Synthetic DNA  17-Amy 1312
cacattac

SEQ ID NO: 145 - DNA, 8 bp, Artificial Sequence
  Synthetic DNA  17-Amy 1321
gattccag

SEQ ID NO: 146 - DNA, 8 bp, Artificial Sequence
  Synthetic DNA  17-Amy 1331
tccgcggt

SEQ ID NO: 147 - DNA, 8 bp, Artificial Sequence
  Synthetic DNA  17-Amy 1343
actgacga

SEQ ID NO: 148 - DNA, 8 bp, Artificial Sequence
  Synthetic DNA  17-Amy 1351
gcacacat

SEQ ID NO: 149 - DNA, 8 bp, Artificial Sequence
  Synthetic DNA  17-Amy 1354
cctaggat

SEQ ID NO: 150 - DNA, 8 bp, Artificial Sequence
  Synthetic DNA  17-Amy 1356
cttaacga

SEQ ID NO: 151 - DNA, 8 bp, Artificial Sequence
  Synthetic DNA  17-Amy_1357
ccaccatc

SEQ ID NO: 152 - DNA, 8 bp, Artificial Sequence
  Synthetic DNA  17-Amy 1359
tgagccgc

SEQ ID NO: 153 - DNA, 8 bp, Artificial Sequence
  Synthetic DNA  17-Amy 1366
tactccac

SEQ ID NO: 154 - DNA, 8 bp, Artificial Sequence
  Synthetic DNA  17-Amy_1369
ggcagccg

SEQ ID NO: 155 - DNA, 8 bp, Artificial Sequence
  Synthetic DNA  17-Amy 1375
ttcgactc

SEQ ID NO: 156 - DNA, 8 bp, Artificial Sequence
  Synthetic DNA  17-Amy 1386
tacgaata

SEQ ID NO: 157 - DNA, 8 bp, Artificial Sequence
  Synthetic DNA  17-Amy 1392
aggtcctt

SEQ ID NO: 158 - DNA, 8 bp, Artificial Sequence
  Synthetic DNA  17-Amy 1397
cagcgagg

SEQ ID NO: 159 - DNA, 8 bp, Artificial Sequence
  Synthetic DNA  17-Amy 1398
gacctcag

SEQ ID NO: 160 - DNA, 8 bp, Artificial Sequence
  Synthetic DNA  17-Amy 1408
gctaggcg

SEQ ID NO: 161 - DNA, 8 bp, Artificial Sequence
  Synthetic DNA  17-Amy 1414
tgcacgga

SEQ ID NO: 162 - DNA, 8 bp, Artificial Sequence
  Synthetic DNA  17-Amy 1427
taacgacc

SEQ ID NO: 163 - DNA, 8 bp, Artificial Sequence
  Synthetic DNA  17-Amy 1436
aacggttc

SEQ ID NO: 164 - DNA, 8 bp, Artificial Sequence
  Synthetic DNA  17-Amy 1437
tgcaatgc

SEQ ID NO: 165 - DNA, 8 bp, Artificial Sequence
  Synthetic DNA  17-Amy_1439
attcgagc

SEQ ID NO: 166 - DNA, 8 bp, Artificial Sequence
  Synthetic DNA  17-Amy_1440
tgcgttcc

SEQ ID NO: 167 - DNA, 8 bp, Artificial Sequence
  Synthetic DNA  17-Amy 1447
atgatcca

SEQ ID NO: 168 - DNA, 8 bp, Artificial Sequence
  Synthetic DNA  17-Amy 1448
ggaacgat

SEQ ID NO: 169 - DNA, 8 bp, Artificial Sequence
  Synthetic DNA  17-Amy_1451
tccgaagc

SEQ ID NO: 170 - DNA, 8 bp, Artificial Sequence
  Synthetic DNA  17-Amy 1453
ctgccaac

SEQ ID NO: 171 - DNA, 8 bp, Artificial Sequence
  Synthetic DNA  17-Amy 1462
aaccgcgg

SEQ ID NO: 172 - DNA, 8 bp, Artificial Sequence
  Synthetic DNA  17-Amy 1466
agagcgag

SEQ ID NO: 173 - DNA, 8 bp, Artificial Sequence
  Synthetic DNA  17-Amy 1470
tcgtatgt

SEQ ID NO: 174 - DNA, 8 bp, Artificial Sequence
  Synthetic DNA  17-Amy 1473
ctcgcttc

SEQ ID NO: 175 - DNA, 8 bp, Artificial Sequence
  Synthetic DNA  17-Amy 1490
tggagcgc

SEQ ID NO: 176 - DNA, 8 bp, Artificial Sequence
  Synthetic DNA  17-Amy 1491
gtggccgt

SEQ ID NO: 177 - DNA, 8 bp, Artificial Sequence
  Synthetic DNA  17-Amy 1493
tggccacc

SEQ ID NO: 178 - DNA, 8 bp, Artificial Sequence
  Synthetic DNA  17-Amy 1506
gcgcagtt

SEQ ID NO: 179 - DNA, 8 bp, Artificial Sequence
  Synthetic DNA  17-Amy_1507
tctccgta

SEQ ID NO: 180 - DNA, 8 bp, Artificial Sequence
  Synthetic DNA  17-Amy 1509
gcgttgcg

SEQ ID NO: 181 - DNA, 8 bp, Artificial Sequence
  Synthetic DNA  17-Amy 1511
gatagcat

SEQ ID NO: 182 - DNA, 8 bp, Artificial Sequence
  Synthetic DNA  17-Amy 1512
aaccaggt

SEQ ID NO: 183 - DNA, 8 bp, Artificial Sequence
  Synthetic DNA  17-Amy_1536
catgacta

SEQ ID NO: 184 - DNA, 8 bp, Artificial Sequence
  Synthetic DNA  17-Amy 1541
gtctcgga

SEQ ID NO: 185 - DNA, 8 bp, Artificial Sequence
  Synthetic DNA  17-Amy 1543
ctctaagt

SEQ ID NO: 186 - DNA, 8 bp, Artificial Sequence
  Synthetic DNA  17-Amy 1560
catcgtgt

SEQ ID NO: 187 - DNA, 8 bp, Artificial Sequence
  Synthetic DNA  17-Amy 1574
gcaacctt

SEQ ID NO: 188 - DNA, 8 bp, Artificial Sequence
  Synthetic DNA  17-Amy 1577
gagattct

SEQ ID NO: 189 - DNA, 8 bp, Artificial Sequence
  Synthetic DNA  17-Amy 1586
cactgctt

SEQ ID NO: 190 - DNA, 8 bp, Artificial Sequence
  Synthetic DNA  17-Amy 1621
aggtacga

SEQ ID NO: 191 - DNA, 8 bp, Artificial Sequence
  Synthetic DNA  17-Amy  1635
accgagtc

SEQ ID NO: 192 - DNA, 8 bp, Artificial Sequence
  Synthetic DNA  17-Amy  1645
cacaagta

SEQ ID NO: 193 - DNA, 20 bp, Artificial Sequence
  Synthetic DNA
aatgatacggcgaccaccga

SEQ ID NO: 194 - DNA, 21 bp, Artificial Sequence
  Synthetic DNA
caagcagaagacggcatacga

SEQ ID NO: 195 - DNA, 51 bp, Artificial Sequence
  Synthetic DNA
  misc_feature (30)..(37): n is a, c, g, or t
aatgatacggcgaccaccgagatctacacnnnnnnnntcgtcggcagcgtc

SEQ ID NO: 196 - DNA, 47 bp, Artificial Sequence
  Synthetic DNA
  misc_feature (25)..(32): n is a, c, g, or t
caagcagaagacggcatacgagatnnnnnnnngtctcgtgggctcgg

(19) United States
(12) Patent Application Publication
(10) Pub. No.: US 2018/0127804 A1
(43) Pub. Date: May 10, 2018

(54) HIGH-THROUGHPUT SEQUENCING OF POLYNUCLEOTIDES

(71) Applicant: AMYRIS, INC., Emeryville, CA (US)

(72) Inventors: Erik Jedediah DEAN, Emeryville, CA (US); Victor HOLMES, Emeryville, CA (US); Christopher REEVES, Emeryville, CA (US); Elaine SHAPLAND, Emeryville, CA (US)

(21) Appl. No.: 15/532,865

(22) PCT Filed: Dec. 4, 2015
(86) PCT No.: PCT/US2015/064029

§ 371 Date: Jun. 2, 2017

Related U.S. Application Data
(60) Provisional application No. 62/088,416, filed on Dec. 5, 2014.
(60) Provisional application No. 62/144,174, filed on Apr. 7, 2015.

(51) Int. Cl. C12Q 1/68; C12N 15/10; C12N 15/66

(52) U.S. Cl. CPC C12Q 1/6806; C12N 15/1093; C12N 15/66; C12Q 2521/507; C12Q 2525/191; C12Q 2535/122; C12Q 2563/179; C12N 15/1003

(57) ABSTRACT

Provided herein are methods, compositions, and kits for simultaneously sequencing polynucleotides from a plurality of samples in a single sequencing run. In an embodiment, the present invention improves efficiency of the next-generation sequencing process, in part, by reducing reaction volumes to a sub-microliter range and generating and using a set of novel barcode sequences to tag a plurality of polynucleotides. In addition, the sample preparation processes have been simplified to save time and cost, while providing high-quality sequence coverage for all samples.

### CROSS REFERENCE TO RELATED APPLICATION

This application claims priority to U.S. Provisional Patent Application No. 62/088,416 filed Dec. 5, 2014 and U.S. Provisional Patent Application No. 62/144,174, filed Apr. 7, 2015, which are incorporated herein by reference.

### U.S. GOVERNMENT LICENSE RIGHTS

This invention was made with Government support under Agreement HR0011-12-3-0006, awarded by DARPA. The Government has certain rights in the invention.

### FIELD OF THE INVENTION

The methods and compositions provided herein generally relate to the fields of molecular biology and genetic engineering.

### BACKGROUND

Synthetic biologists routinely assemble well-characterized DNA parts into larger constructs and introduce those DNA assemblies into host organisms to achieve desired phenotypes. See Weenink and Ellis (2013) Methods Mol. Biol. 1073: 51-60; Polizzi (2013) Methods Mol. Biol. 1073: 3-6; Munnelly (2013) ACS Synth Biol. 2: 213-215; Stephanopoulos (2012) ACS Synth. Biol. 1: 514-525. This is often a trial-and-error process that requires building and testing tens to thousands of DNA assemblies. For example, a comprehensive combinatorial exploration of five genes each expressed at five levels would require 3125 DNA assemblies. At synthetic biology companies, it is common to build many constructs to test diverse hypotheses or to optimize a multi-gene pathway using iterative design-build-test-learn cycles similar to strategies described previously. See Gardner et al. (U.S. Pat. Nos. 8,859,261; 8,415,136); Du et al. (2014) ACS Chem. Biol. 9: 2748-2754; Ajikumar et al. (2010) Science 330: 70-74. At this scale, quality control (QC) of large numbers of DNA assemblies creates logistical and economic challenges.

High-throughput strain engineering facilities routinely use automated workflows to assemble thousands of DNA constructs ranging in size from 3-30 kb and containing 2-12 DNA parts. The DNA assemblies must hence undergo rigorous QC to avoid building and testing incorrectly engineered strains, which could lead to erroneous conclusions regarding genotype-phenotype relationships. Because no assembly method is perfect, finding a correct assembly requires QC analysis to be performed on multiple clones. Until recently, this involved comparing the observed restriction endonuclease fragment sizes to those computationally predicted for four colonies, followed by Sanger sequencing of the chosen clone. To achieve 2× coverage across a 10 kb assembly using Sanger sequencing requires at least 24 reads spaced appropriately across the assembly and costs at least $72 at present day value. This is too expensive and logistically onerous for a high throughput operation.

Next-generation sequencing (NGS) technology has greatly reduced the cost of sequencing whole genomes, but its application for the simultaneous sequencing of multiple plasmid constructs or other smaller size DNA constructs has been limited. Thus, there remains a need for high-throughput, low-cost sequencing methods for less than genome-scale applications.

### SUMMARY

Provided herein are methods, compositions, and kits for preparing and simultaneously sequencing a plurality of polynucleotides (e.g., plasmids comprising DNA assemblies) in a single sequencing run of a sequencing instrument. In certain embodiments, a next-generation sequencing platform is combined with an acoustic liquid handling instrument to provide a rigorous, low-cost QC method that enables complete sequencing of almost every DNA assembly built by a high throughput operation. Embodiments of the present invention increase the efficiency of sequencing operations by simplifying workflow and reducing cost and hands-on time to perform experiments, as compared to known sequencing methods. The Illumina MiSeq sequencer can provide about 5 gigabases (GB) of data in a 24 hour run using the 300-cycle v2 kit (Perkins et al. (2013) PLoS One 8: e67539; Loman et al. (2012) Nat. Biotechnol. 30: 434-439), theoretically allowing 25,000 plasmids of 10 kb average size to be sequenced. However, there were several obstacles to overcome before even a fraction of this high level of multiplexing can be achieved.

The Illumina Nextera method for preparing sequencing libraries is convenient and robust (Caruccio (2011) Methods Mol. Biol. 733: 241-255). However, cost-effective sequencing of plasmids in the 3 to 30 kb range requires hundreds of barcode primers and a significant reduction in the use of the expensive Nextera reagents. A recent report described a Nextera workflow in which reaction volumes were reduced eight-fold relative to the Illumina protocol (Lamble (2013) BMC Biotechnol. 13: 104). Here, in addition to showing that the volume of the tagmentation reaction can be reduced 100-fold using acoustic droplet ejection, it has been demonstrated that thousands of uniquely barcoded samples can be handled with the appropriate automation infrastructure. It has also been demonstrated that over 4000 plasmids with an average size of 8 kb (largest about 20 kb) can be simultaneously sequenced at a consumables cost of less than $3 per plasmid. Furthermore, embodiments of the present invention include systems and software to track the samples and associated sequence data and to rapidly identify correctly assembled constructs having the fewest defects. This NGS quality control (QC) process should be of value to any group operating a high-throughput molecular biology pipeline.

Thus, in one aspect, provided herein is a method of preparing a plurality of polynucleotides for simultaneous sequencing. The method comprises, for each input polynucleotide of a plurality of input polynucleotides, (a) amplifying the input polynucleotide by rolling circle amplification (RCA) in an RCA solution to generate a target polynucleotide; (b) diluting the RCA solution comprising the target polynucleotide by a standard dilution factor; (c) generating a reaction mixture having a volume of about 0.005 μL to about 2 μL and comprising tagged polynucleotide fragments by contacting the diluted RCA solution comprising the target polynucleotide with transposases pre-loaded with transposon end sequences to fragment and tag the target polynucleotide; (d) removing the transposases from the tagged polynucleotide fragments, thereby generating a reaction solution; and (e) performing a polymerase chain reaction (PCR) with the reaction solution comprising the tagged polynucleotide fragments, wherein the PCR utilizes adapter primers comprising barcode sequences that are capable of hybridizing to the tagged polynucleotide fragments to generate barcoded polynucleotide fragments.

In one embodiment, the method further comprises: (f) combining the barcoded polynucleotide fragments generated for each input polynucleotide of the plurality of input polynucleotides; (g) sequencing the combined barcoded polynucleotide fragments in step (f) in a single sequencing run to generate sequence reads; (h) sorting the sequence reads from the sequencing run using the barcode sequences associated with each input polynucleotide; and (i) aligning and assembling the sequence reads for each input polynucleotide to generate a consensus sequence of the input polynucleotide.

In another embodiment, the barcode sequences are selected from the group consisting of SEQ ID NO: 1 through SEQ ID NO: 192.

In another embodiment, the plurality of input polynucleotides is at least 1000, at least 2000, at least 3000, or at least 4000.

In another embodiment, the input polynucleotide is a plasmid DNA.

In another embodiment, the input polynucleotide comprises a DNA assembly of a plurality of DNA components.

In another embodiment, the input polynucleotide is a plasmid and the combined barcoded polynucleotide fragments are generated from at least 1000 plasmids.

In another embodiment, the input polynucleotide is a plasmid and the combined barcoded polynucleotide fragments are generated from at least 4000 plasmids.

In another embodiment, less than 2 percent of the plasmids had less than 15 times average sequencing coverage.

In another embodiment, the reaction mixture has a volume of about 0.5 μL. In another embodiment, the reaction mixture has a volume of less than about 1 μL. In another embodiment, the reaction mixture has a volume of less than about 2 μL.

In another embodiment, the standard dilution factor is determined by: (a) measuring a concentration of the target polynucleotide in the RCA solution for at least a portion of the plurality of input polynucleotides; (b) determining an average concentration of the target polynucleotides in the RCA solution for the at least the portion of the plurality of input polynucleotides; and (c) calculating the standard dilution factor by dividing the average concentration by 5 ng/μL.

In another embodiment, the diluted RCA solution comprises the target polynucleotide at a concentration between about 3 ng/μL and about 10 ng/μL.

In another embodiment, the transposases are removed from the tagged polynucleotide fragments by treating the reaction mixture from step (c) under a dissociation condition.

In another embodiment, the treating the reaction mixture from step (c) under the dissociation condition comprises adding a dissociation solution to the reaction mixture.

In another embodiment, the dissociation solution comprises sodium dodecyl sulfate (SDS). In another embodiment, a concentration of the SDS in the reaction solution is between about 0.05% to about 0.3%.

In another embodiment, the dissociation solution comprises sodium dodecyl sulfate (SDS) and a concentration of the SDS in the reaction solution is about 0.1%.

In another embodiment, the method further comprises diluting the reaction solution by at least 10-fold with an aqueous solution prior performing the PCR.

In another embodiment, the transposases are removed from the tagged polynucleotide fragments without using solid phase extraction or centrifugation.

In another embodiment, the method further comprises, after the PCR, (f) removing small polynucleotide fragments from PCR products; (g) quantifying a concentration of the barcoded polynucleotide fragments from step (f) for each input polynucleotide; and (h) determining a volume of the barcoded polynucleotide fragments in step (f) to add to a pool assuming an average polynucleotide fragment size of 500 base pairs and normalizing for a length of the input polynucleotide.

In another embodiment, the method further comprises filtering the combined barcoded polynucleotide fragments to remove small fragments having a size less than about 300 base pairs.

In another aspect, provided herein is a method of preparing a plurality of polynucleotides for sequencing, the method comprising: (a) generating a reaction mixture having a volume of about 0.005 μL to about 2 μL and comprising tagged polynucleotide fragments by contacting a target polynucleotide with transposases pre-loaded with transposon end sequences to fragment and tag the target polynucleotide; and (b) performing a polymerase chain reaction (PCR) with a reaction solution comprising the reaction mixture comprising the tagged polynucleotide fragments and adapter primers comprising barcode sequences capable of hybridizing to the tagged polynucleotide fragments to generate barcoded polynucleotide fragments.

In one embodiment, the method further comprises: (c) repeating steps (a) and (b) described above to generate barcoded polynucleotide fragments from a plurality of target polynucleotides, wherein the barcoded polynucleotide fragments from each of the plurality of target polynucleotides comprise a unique barcode sequence; (d) combining the barcoded polynucleotide fragments generated from the plurality of target polynucleotides; and (e) sequencing the combined barcoded polynucleotide fragments in a single sequencing run to generate sequence reads.

In another aspect, provided herein is a method of preparing a plurality of polynucleotides for sequencing, the method comprising: for each input polynucleotide of a plurality of input polynucleotides, (a) amplifying the input polynucleotide by rolling circle amplification (RCA) in an RCA solution to generate a target polynucleotide; (b) diluting the RCA solution comprising the target polynucleotide by a standard dilution factor; (c) generating a reaction mixture having a volume of about 0.005 μL to about 2 μL and comprising tagged polynucleotide fragments by contacting the diluted RCA solution comprising the target polynucleotide with transposases pre-loaded with transposon end sequences to fragment and tag the target polynucleotide; (d) adding a dissociation solution to the reaction mixture to remove the transposases from the tagged polynucleotide fragments, thereby generating a reaction solution; (e) diluting the reaction solution with an aqueous solution; (f) adding to the diluted reaction solution a pair of adapter primers comprising barcode sequences capable of hybridizing to the tagged polynucleotide fragments; (g) performing a polymerase chain reaction (PCR) with the diluted reaction solution and terminal primers to generate barcoded polynucleotide fragments, wherein the terminal primers are capable of hybridizing to the barcoded polynucleotide fragments; (h) combining the barcoded polynucleotide fragments generated in step (g) for each input polynucleotide of the plurality of input polynucleotides; (i) sequencing the combined barcoded polynucleotide fragments of step (h) in a single sequencing run to generate sequence reads; (j) sorting the sequence reads from the sequencing using the barcode sequences associated with each input polynucleotide to assign each of the sequence reads to each input polynucleotide; and (k) aligning and assembling the sorted sequence reads for each of the input polynucleotide to generate a consensus sequence of each input polynucleotide.

In certain embodiments, the reaction mixture is generated using an acoustic liquid handling instrument.

In another aspect, provided herein is a kit comprising: (a) a plurality of barcoded adapter primers produced by the method described herein; and (b) reagents to perform polymerase chain reaction. In certain embodiments, the kit comprises at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, or at least 190 different adapter primers.

In an embodiment, the barcode sequences may be selected from the group consisting of SEQ ID NO: 1 to SEQ ID NO: 192.

In another embodiment, the barcoded polynucleotide fragments comprise combined barcoded polynucleotide fragments generated from a plurality of target polynucleotides, and wherein the barcoded polynucleotide fragments from each of the plurality of target polynucleotides comprise a first barcode sequence selected from the group consisting of SEQ ID NO: 1-96 and a second barcode sequence selected from the group consisting of SEQ ID NO: 97-192.

In another aspect, provided herein is a composition comprising a library of barcoded polynucleotide fragments comprising a barcode sequence produced by the method described herein. In an embodiment, the barcode sequences may be selected from the group consisting of SEQ ID NO: 1 to SEQ ID NO: 192. In certain embodiments, the plurality of target polynucleotides are generated from at least 1000, at least 2000, at least 3000, or at least 4000 samples of plasmid DNA.

### BRIEF DESCRIPTION OF THE FIGURES

FIG. 1 illustrates the reactions involved in sequencing library generation using the tagmentation process. A mixture of transposomes carrying two different sequences inserts those sequences into a target DNA, a process known as tagmentation. After removing the transposases from the DNA, fragment ends are repaired and a few cycles of polymerase chain reaction (PCR) are used to attach additional sequences required for multiplex sequencing.

FIG. 2 illustrates a schematic diagram of the next-generation sequencing quality control workflow according to an embodiment of the present invention. The type of liquid dispenser robot system used at each step according to one embodiment is indicated in the parenthesis.

FIG. 3A illustrates distribution and statistics of read coverage for 768 samples prepared from DNA of 384 plasmids prepared by rolling circle amplification (RCA) (diamonds—a lower curve) or miniprep (MP; squares—an upper curve) according to an embodiment of the present invention. The horizontal line that meets at the y-axis indicates the 15× coverage threshold. MAD is the median absolute deviation.

FIG. 3B illustrates the comparison of DNA size ranges for RCA prepared nucleic acids that are normalized versus not normalized according to an embodiment of the present invention. The size distributions of RCA DNA that had been normalized before tagmentation were very similar to those that had not been normalized. This suggests that DNA amplified by RCA is of even concentration across many samples.

FIG. 4 illustrates the effect of RCA DNA concentration in the tagmentation reactions on the percentage of reads assigned based on the barcodes according to an embodiment of the present invention. Each point represents the average of 48 samples; error bars are standard deviation. The expected average for the 384 samples is 0.26%.

FIG. 5 illustrates the distribution of read coverage and statistics for a run containing 4078 plasmid samples according to an embodiment of the present invention.

FIG. 6 illustrates exemplary sequence data plots for samples from the run of 4078 samples according to an embodiment of the present invention. The numbers in thousands along the x-axis on the top of each sequence data plot represent nucleotide positions. The numbers along the y-axis on the left of each sequence data plot represent read coverage depth. The top two sequence data plots (D17736 and D17985) show samples with differences between the reads and the reference, while the bottom two sequence data plots (D17804 and D21147) show samples that match the reference perfectly (not counting the vector portions). The green region shows the depth of coverage (represented by an area underneath jagged lines). Red and blue vertical bars along the x-axis indicate a single nucleotide polymorphism (SNP) in the forward and reverse reads. Purple and yellow vertical bars along the x-axis indicate an indel in the forward and reverse reads. Note that even with less than 15× average coverage (bottom right sequence data plot D21147), it is sometimes possible to obtain reliable QC data. At the bottom of each plot are the DNA assembled parts in green (shown as blank horizontal bars along the x-axis—e.g., R39309 for plot D17736; R40174 and R2663 for plot D17985; R40200 and R2663 for plot D17804; and R29189, R20770, R39300, and R2662 for plot D21147) and the vector portions in yellow (shown as hatched bars along the x-axis—e.g., V25745R and V25745L for all four sequence data plots). In these exemplary embodiments, different DNA parts and vector portions are joined using linkers.

FIG. 7A illustrates optimum SDS and Triton X-100 concentrations for removal of the transposase after tagmentation according to an embodiment of the present invention. Shown in FIG. 7A is a response surface plot of the concentration of DNA amplified by PCR relative to that obtained using Zymo column purification. The DNA concentration in a selected size range was determined using a Bioanalyzer. SDS was added to the tagmentation reaction to different final concentrations, as shown along the horizontal axis, followed after 10 minutes at 75° C. by dilution with TritonX-100 solutions giving concentrations between 0 and 2%, as shown along the vertical axis. The black dots are the actual data points specified by the design of experiment using JMP (SAS Institute, Inc., Cary, N.C.). The maximum recovery was found to be 57% of the Zymo column control at 0.1% SDS, 0% Triton. It was later found that heating to 75° C. was unnecessary.

FIGS. 7B1 through 7B3 illustrate superimposed fragment analyzer traces of samples treated with the Zymo kit, with 0.2% SDS final concentration, or with 0.1% SDS final concentration. All samples were incubated at room temperature. DNA fragment size is shown along the horizontal axis and DNA concentration is shown along the vertical axis (RFU=Relative Fluorescence Units). Zymo-treated samples have the majority of fragments (by moles) below 600 base pairs. SDS-treated samples have the majority of fragments (by moles) above 600 base pairs.

FIG. 8 illustrates PCR efficiency using Vent polymerase and primers ordered from IDT or the Nextera kit reagents NPM and PPC according to an embodiment of the present invention. The template was tagmented DNA following the Illumina Nextera kit protocol. PCR efficiency is defined as ([DNA]_{final}/[DNA]ᵢₙᵢₜᵢₐₗ)(1/N), where N is the number of cycles of PCR. Perfect efficiency is 2 and no amplification is 1. The concentration of DNA in a chosen size range before and after PCR was measured with a Bioanalyzer 2100 and a high sensitivity chip.

FIG. 9 illustrates a demonstration of transfer of RCA DNA by the Echo acoustic liquid transfer system according to an embodiment of the present invention. A source plate containing precise concentrations of DNA prepared by RCA of a single plasmid construct (actual ng/μL) was used to transfer one μL to the same wells of a low volume black assay plate (Costar 3677) on the Echo. The amount of transferred DNA was then assayed by Picogreen fluorescence. For each data point N=48 and the error bars are standard deviation.

FIG. 10 illustrates correlation of read coverage comparing two separate MiSeq runs of the same plasmids prepared for sequencing by the protocol according to an embodiment of the present invention.

FIG. 11A is a schematic diagram showing a flowchart of designing barcode sequences and barcoded adapter primers according to an embodiment of the present invention.

FIG. 11B is a schematic diagram illustrating a flowchart for analyzing sequence data according to an embodiment of the present invention.

FIG. 12 is a schematic diagram showing a computer system according to an embodiment of the present invention.

### DETAILED DESCRIPTION OF THE EMBODIMENTS

The rapid growth in the field of synthetic biology over the last decade has been driven in large part by advances in the synthesis and sequencing of DNA sequences. A decade ago, synthesizing DNA, such as simple oligonucleotides, was tedious and could cost hundreds of dollars, but today these DNA parts are ordered automatically and delivered next-day for tens of dollars. The DNA sequencing technology has also progressed, particularly through the extensive automation and scaling of Sanger sequencing technology. However, the progress in DNA sequencing technology has lagged behind DNA synthesis technology and has become cost-limiting for many researchers in this field.

Recent commercialization of so-called next-generation sequencing technologies promise to overcome this lag and dramatically increase the amount of DNA read per dollar. Next-generation sequencing technologies include instruments capable of parallelizing the sequencing process, producing thousands or millions of sequence reads concurrently per instrument run. For genome-size DNA templates, this promise of increasing the amount of DNA read per dollar has been fulfilled by commercially available kits. For smaller size DNA samples, such as plasmid DNA, no workflow has yet been developed that can reap the cost benefits of next-generation sequencing.

The methods, compositions, and kits provided herein improve the efficiency of next-generation sequencing process for samples with input polynucleotides having a small size (e.g., 3-30 kb range) by increasing sample throughput, simplifying workflow, and decreasing the cost. The compositions and methods described herein bridges the power of next-generation sequencing to the plasmid libraries and other smaller size DNAs used in gene synthesis, DNA assembly, enzyme engineering, amplicon sequencing, library deconvolution, and the like. Here, the efficiency of sequencing workflow has improved dramatically, in part, due to reducing sample reaction volumes and reducing the amount of key reagents for each reaction. As a result, the cost of sample preparation is significantly reduced. Furthermore, by increasing the number of samples combined into a single sequencing run, the throughput of sample processing is significantly increased. In particular, there are three main aspects of the present invention that contribute to low-cost, high-throughput processing of thousands of samples.

In one aspect, methods and compositions described herein can provide at least 100-fold reduction in reaction volume for a standard DNA tagmentation reaction. By using an acoustic liquid transfer system, a reaction usually performed at a volume of 50 μL can be reduced down to a volume of 2 μL or less, or even to a volume of about 0.5 μL. The second and third aspects of the invention have been developed to further accommodate this small reaction volume.

In another aspect, the methods and compositions described herein provide concomitant reduction in volume of both target polynucleotide derived from a sample and tagmentation enzyme to reduce overall cost of the reaction. The decreased polynucleotide concentration can be compensated for by increasing the number of cycles in the subsequent PCR step. Although a shift in the size distribution of DNA fragments is observed with increasing PCR cycles, no significant change in sequence quality was observed due to the reduction in a reaction volume during tagmentation.

In another aspect, the methods and compositions described herein provide novel barcode sequences, which increase the number of samples that can be combined together into a single sequencing run. These barcode sequences also decrease the sequencing cost and provide higher throughput, as fewer sequencing runs are required to sequence a large number of samples.

By utilizing the above described and other features of methods and compositions described herein, a workflow has been developed so that a high-quality sequence coverage can be provided for thousands of samples per week. Such high quality sequence coverage can be provided at a reasonable cost, for example, less than $3 per plasmid at present day value. This cost represents more than a 25-fold reduction over the alternative Sanger sequencing technology. The compositions and methods provided herein provide many advantages in the field of synthetic biology as well as other technical areas. These and other aspects of the present invention are described more fully throughout the specification below.

### Definitions

As used herein, the term “transposon” refers to a nucleic acid segment, which is recognized by a transposase and which is a component of a functional nucleic acid-protein complex (i.e., a transposome or transposition complex) capable of transposition.

As used herein, the term “transposase” or “fragmentation and labeling enzyme” refers to an enzyme, which is a component of a functional nucleic acid-protein complex capable of transposition and which is mediating transposition.

As used herein, the term “transposon end” or “transposon end sequence” refers to a double stranded DNA that exhibits nucleotide sequences that are necessary to form the complex with the transposase enzyme that is functional in an in vitro transposition reaction. The transposon end sequences are responsible for identifying the transposon for transposition. A transposon end forms a transposome or transposition complex with a transposase to perform transposition reaction. In certain embodiments, the transposon end sequence may further include additional sequences such as primer binding sites or other functional sequences.

As used herein, the term “transposome” or “transposition complexes” refers to the formation between a transposase enzyme and a fragment of double stranded DNA that contains a specific binding sequence of the enzyme, termed “transposon end.” The complex formed between a transposase enzyme and transposon end capable of mediating transposition and fragmentation of a target polynucleotide is also referred to as transposases “pre-loaded” with transposon end sequences.

As used herein, the term “rolling circle amplification” refers to nucleic acid amplification reactions where a circular nucleic acid template is replicated in a single long strand with tandem repeats of the sequence of the circular template. This first, directly produced tandem repeat strand is referred to as tandem sequence DNA and its production is referred to as rolling circle replication. Rolling circle amplification refers to both to rolling circle replication and to processes involving both rolling circle replication and additional forms of amplification.

As used herein, the term “amplification” refers to a method or process that increases the representation of a population of specific nucleotide sequences in a sample.

As used herein, the term “standard dilution factor” refers to a number that is used to uniformly dilute all solutions comprising target polynucleotides to be simultaneously sequenced. For example, all solutions comprising target polynucleotides may be diluted by a “standard dilution factor” of 1:5 by adding 20 μL of water to 5 μL of each of the solutions, regardless of the concentration of DNA in each solution.

The terms “nucleic acid” or “polynucleotide” refers to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically, or biochemically modified, non-natural, or derivatized nucleotide bases.

As used herein, the term “input polynucleotide” can refer to a nucleic acid molecule from a sample of interest and/or a known nucleic acid sequence, and it may be a source material for generating a target polynucleotide.

As used herein, the terms “target polynucleotide” or “target DNA” may be used to refer to nucleic acid molecules that are derived from an input polynucleotide. The target polynucleotide or target DNA may be subject to fragmentation and/or tagging with adapters and/or barcode sequences. The target polynucleotide may be essentially any nucleic acid of known or unknown sequence. For example, the target polynucleotide may be prepared from a plasmid containing a DNA assembly of known genes and other functional elements. If rolling circle amplification is used to prepare a sample, then the target polynucleotide may include tandem repeats of the sequence of the circular template, such as a plasmid. In some embodiments, a target polynucleotide may include sequences of a vector and a polynucleotide insert (e.g., a DNA assembly).

In an embodiment, an input polynucleotide and a target polynucleotide may be the same. For example, if a plasmid mini-preparation procedure is used to amplify and isolate plasmid DNA, then an input polynucleotide (i.e., a plasmid) and target polynucleotide (i.e., a plasmid) generated from the mini-preparation may be the same. In another embodiment, an input polynucleotide and a target polynucleotide may be different. For example, if a plasmid DNA is subject to rolling circle amplification to generate a concatemer of a plasmid DNA, then the initial plasmid DNA may be referred to as an input polynucleotide, and the concatemer of the plasmid DNA, which is subject to fragmentation and tagging, is referred to a target polynucleotide.

As used herein, the term “sample” generally refers to anything capable of being analyzed by the methods provided herein that contains an input polynucleotide, a target polynucleotide, or any fragments thereof. In an embodiment, a sample may refer to a source for a particular input polynucleotide and/or target polynucleotide. For example, two plasmids comprising two different DNA assemblies may be referred to as two different samples. In some embodiments, replicates or clones comprising the same plasmid DNA may be referred to as separate samples.

As used herein, the term “consensus sequence” is a sequence determined after alignment of sequence reads associated with an input polynucleotide or a target polynucleotide generated from a sequencer by determining the base which is the most commonly found at each position in the compared, aligned sequence reads.

As used herein, the term “tagged DNA fragment,” “tagmented DNA fragment,” “tagged polynucleotide,” or “tagmented polynucleotide” refers to a piece of DNA or polynucleotide which has been fragmented and tagged or appended with one or more additional components, such as a transposon end sequence. In an embodiment, the tagged DNA fragment or tagged polynucleotide fragment may be generated during a tagmentation reaction while incubating a target DNA or a target polynucleotide with transposomes or transposition complexes.

As used herein, the term “tagmentation reaction” refers to incubation of a target polynucleotide with transposomes or transposition complexes to tag and fragment the target polynucleotide with transposon ends.

As used herein, the term “tagmentation reaction mixture” refers to a reaction mixture that includes a mixture of tagged polynucleotide fragments, transposases, unreacted components of a tagmentation reaction, and other components generated from a tagmentation reaction. The term “reaction mixture” is also used herein to refer to a “tagmentation reaction mixture,” and any discussions related to a tagmentation reaction mixture provided herein also applies to a reaction mixture.

As used herein, the term “tagmentation reaction solution” refers to a reaction solution comprising the tagmentation reaction mixture that has been treated under a dissociation condition to remove transposases from tagged polynucleotide fragments. The term “reaction solution” is also used herein to refer to a “tagmentation reaction solution,” and any discussions related to a tagmentation reaction solution provided herein also applies to a reaction solution.

As used herein, the term “dissociation condition” refers to a condition that can be used to treat the tagmentation reaction mixture to dissociate or remove transposases from tagged polynucleotide fragments generated from a tagmentation reaction. The dissociation condition can include, for example, treatment with heat or adding a solution, such as a dissociation or denaturing solution comprising a surfactant, which promote transposases to become unbound from tagged polynucleotide fragments.

As used herein, the term “primer” refers to a polynucleotide sequence that is capable of specifically hybridizing to a polynucleotide template sequence, e.g., a primer binding segment, and is capable of providing a point of initiation for synthesis of a complementary polynucleotide under conditions suitable for synthesis, i.e., in the presence of nucleotides and an agent that catalyzes the synthesis reaction (e.g., a DNA polymerase). The primer is complementary to the polynucleotide template sequence, but it need not be an exact complement of the polynucleotide template sequence. For example, a primer can be at least about 80, 85, 90, 95, 96, 97, 98, or 99% identical to the complement of the polynucleotide template sequence.

As used herein, the term “adapter” refers to a non-target nucleic acid component, generally DNA, which is joined to a target polynucleotide fragment and serves a function in subsequent analysis of the target polynucleotide fragment. In an embodiment, an adapter may include a nucleotide sequence that permits identification, recognition, and/or molecular or biochemical manipulation of the polynucleotide to which the adapter is attached. For example, an adapter may include a sequence which may be used as a primer binding site to read the sequence of the polynucleotide fragments. In another example, an adapter may include a barcode sequence which allows barcoded polynucleotide fragments to be identified.

As used herein, the term “adapter primer” refers to a primer that is capable of specifically hybridizing to a portion of a tagged polynucleotide fragment (e.g., to its primer binding segment, which may include a transposon end sequence), and is capable of providing a point of initiation for synthesis of a complementary polynucleotide under conditions suitable for synthesis. The adapter primer may be used in embodiments of the invention to append an adapter to a tagged polynucleotide fragment to generate a barcoded polynucleotide fragment.

As used herein, the term “barcode sequence” (also referred to as index) may be a known sequence used to associate a polynucleotide fragment with the input polynucleotide or target polynucleotide from which it is produced. It can be a sequence of synthetic nucleotides or natural nucleotides. In some embodiment, a barcode sequence is contained within adapter sequences such that the barcode sequence is contained in the sequencing reads. Each barcode sequence may include at least 4, 5, 6, 7, 8, 9, 10, 11, 12, 13, 14, 15, 16, or more nucleotides in length. In an embodiment, a barcode sequence may include 8 nucleotides in length. Generally, barcode sequences are of sufficient length and sufficiently different from one another to allow the identification of samples based on barcode sequences with which they are associated.

As used herein, “a sample specific barcode sequence” may refer to a barcode sequence specifically used for a particular sample and is different from barcode sequences used for other samples. A sample specific barcode sequence allows the identification of polynucleotide fragments derived from a particular sample (e.g., input or target polynucleotide) from another. In an embodiment, barcoded polynucleotide fragments from each sample may receive a unique combination of two barcode sequences so that sequence reads generated by a sequencer can be assigned to the correct samples (i.e., input polynucleotides) based on the combination of barcode sequences.

As used herein, the term “barcoded adapter primer” refers to an adapter primer which comprises a barcode sequence.

As used herein, the term “tagged polynucleotide fragment” refers to a polynucleotide fragment resulting from a tagmentation reaction. The tagged polynucleotide fragment is “tagged” with transposon end sequences during tagmentation and may further include additional sequences added during extension during a few cycles of PCR.

As used herein, the term “barcoded polynucleotide fragment” refers to a polynucleotide fragment which comprises a barcode sequence. The barcoded polynucleotide fragment may be appended with one or more barcode sequences. The barcoded polynucleotide fragment may be appended with one or more adapters which include barcode sequences.

As used herein, the term polynucleotide “fragment” refers to a polynucleotide including part but not all of the polynucleotide from which it is derived. For example, a polynucleotide fragment may include a piece of a target polynucleotide which is tagmented, cut, or sheared. In some embodiments, a polynucleotide fragment may be generated by amplifying a particular target region from a genome or other sequences.

As used herein, the term “library” refers to a plurality of nucleic acids, and may be used to refer to nucleic acids derived from the same input polynucleotide, target polynucleotide and/or same sample.

As used herein, the term “sequencing run” refers to any step or portion of a sequencing experiment performed to determine some information related to at least one nucleic acid molecule.

As used herein, the term “next-generation sequencing” is a method for sequencing nucleic acid sequences at high speed and at low cost than the previously used Sanger sequencing. The term “next-generation sequencing” platform refers to massive parallel sequencing platforms that allow millions of nucleic acid molecules to be sequenced simultaneously.

A “next-generation sequencer” refers to a sequencer which is capable of next-generation sequencing. A next-generation sequencer can include a number of different sequencers based on different technologies, such as Illumina (Solexa) sequencing, Roche 454 sequencing, Ion torrent sequencing, SOLiD sequencing, and the like.

As used herein, the term “sequence reads” refers to a sequence or data representing a sequence of nucleotide bases, in other words, the order of monomers in a polynucleotide, which is determined by a sequencer.

As used herein, “depth (coverage)” in DNA sequencing refers to the number of times a nucleotide is read during the sequencing process. Deep sequencing indicates that the total number of reads is many times larger than the length of the sequence under study.

As used herein, “average coverage” refers to an average or median of all the per base coverage values. For example, a plasmid with 30× coverage will have an average of 30 reads spanning any given position within the plasmid. Some regions will have higher coverage, and some will have lower coverage. In an embodiment, an average coverage of 15× is set as a threshold to determine the quality of a consensus sequence generated from the sequence reads.

The term “simultaneously” or “concurrently” as used herein refers to any two or more processes that are occurring more or less at the same time. It is not intended that each process begins and ends precisely together, but only that their respective durations may overlap.

The singular forms “a,” “an,” and “the” include plural references unless the context clearly dictates otherwise. Thus, for example, reference to “an adapter primer” includes a single adapter primer as well as a plurality of adapter primers.

### Methods of Preparing Samples and Generating Sequencing Libraries

In one aspect of the invention, provided herein is a method of preparing polynucleotides and generating polynucleotide fragments for highly multiplexed sequencing. The present invention is particularly useful for simultaneously sequencing small-sized input polynucleotides (e.g., about 3 kb to 30 kb range) from hundreds to thousands of samples. The small sized input polynucleotide includes, for example, a plasmid DNA, PCR amplicons, and 16 rRNA. In one embodiment, an input polynucleotide in a sample may be a plasmid DNA comprising an assembled polynucleotide produced by stitching several DNA components. In some embodiments, the assembled polynucleotide in a plasmid may be produced using compositions and methods described in U.S. Pat. Nos. 8,546,136, 8,221,982, and 8,110,360, each of which is incorporated herein by reference in its entirety.

The plurality of input polynucleotides can be processed, combined, and sequenced together in a single sequencing run of a sequencing instrument in a cost effective and time efficient manner. In an embodiment, polynucleotides from many samples (e.g., 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 10100, 10200, 10300, 10400, 10500, 10600, 10700, 10800, 10900, 11000, 11100, 11200, 11300, 11400, 11500, 11600, 11700, 11800, 11900, 12000, 12100, 12200, 12300, 12400, 12500, 12600, 12700, 12800, 12900, 13000, 13100, 13200, 13300, 13400, 13500, 13600, 13700, 13800, 13900, 14000, 14100, 14200, 14300, 14400, 14500, 14600, 14700, 14800, 14900, 15000, 15100, 15200, 15300, 15400, 15500, 15600, 15700, 15800, 15900, 16000, 16100, 16200, 16300, 16400, 16500, 16600, 16700, 16800, 16900, 17000, 17100, 17200, 17300, 17400, 17500, 17600, 17700, 7800, 17900, 18000, 18100, 18200, 18300, 18400, 18500, 18600, 18700, 18800, 18900, 19000, 19100, 19200, 19300, 19400, 19500, 19600, 19700, 19800, 19900, 20000, or more) can be prepared to generate target polynucleotides which are then fragmented and tagged with unique barcode sequences. Thereafter, the barcoded polynucleotide fragments from different samples can be combined together and sequenced in a single sequencing run. The sequence reads generated from the sequencer can then be sorted according to the unique barcode sequences associated with each sample (i.e., input polynucleotide).

In embodiments of the present invention, any suitable methods can be used to tag target polynucleotides with barcode sequences. In one embodiment, target polynucleotides may be initially fragmented because a next-generation sequencer can typically read only about 10 to 1,000 base pairs. Generally, fragmentation can include enzymatic, chemical, or mechanical methods which are well known and available in the art. For example, polynucleotides can be fragmented by acoustic shearing, nebulization, sonication, restriction enzymes, or transposomes. See, e.g., U.S. Patent Application Publication Nos. 2010/0120098 and 2012/0264228. Thereafter, polynucleotide fragments can be appended with one or more adapters at their 5′ and/or 3′ ends, each adapter comprising a unique barcode sequence as well as additional functional sequences. The functional sequences, such as primer binding sites, may be used during subsequent library amplification and sequencing.

Adapters comprising barcode sequences may be attached to polynucleotide fragments using a variety of standard techniques known and available in the art. For example, adapters can be attached to polynucleotide fragments by a ligase or a polymerase. The ligase may be any enzyme capable of ligating an adapter sequence or any oligonucleotide to polynucleotides. Suitable ligases include T4 DNA ligase, which is commercially available. See, e.g., New England Biolas (Ipswich, Mass.). Methods for using ligases are also well known in the art. Exemplary methods are described in, for example, Bentley et al., Nature 456:49-51 (2008); WO 2008/023179; U.S. Pat. No. 7,115,400; and U.S. Patent Application Publication Nos. 2007/0128624; 2009/0226975; 2005/0100900; 2005/0059048; 2007/0110638; and 2007/0128624, each of which is incorporated herein by reference in its entirety.

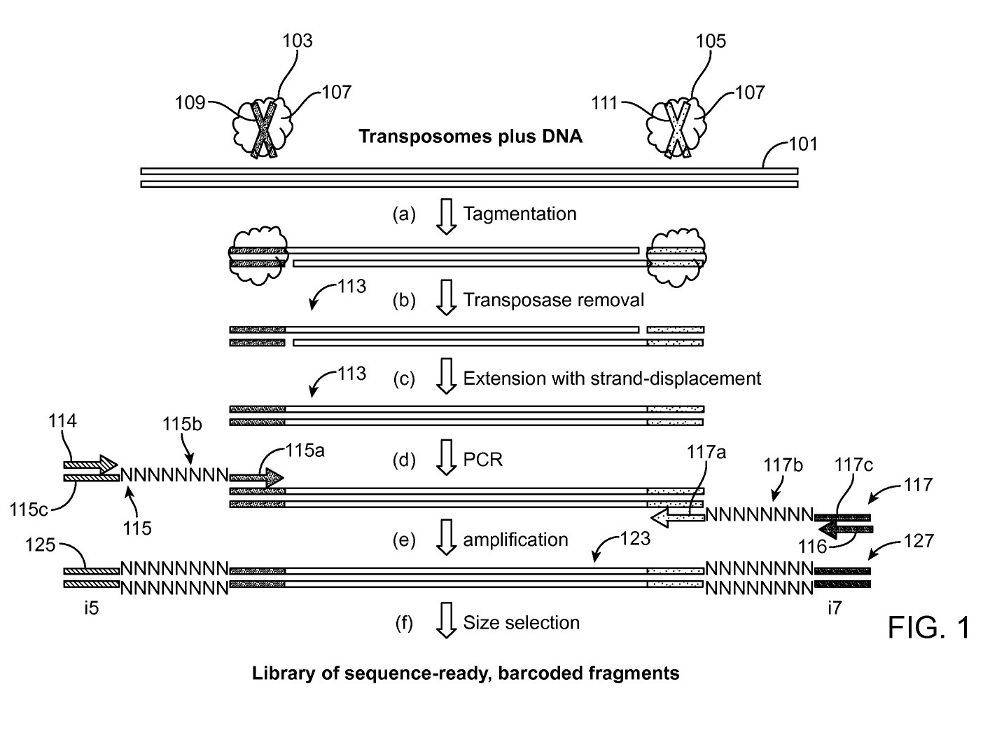

Alternatively, target polynucleotides derived from a sample may be fragmented and adapters may be added to the 5′ and 3′ ends using tagmentation or transposition reactions. The methods for tagmentation or transposition reactions are well-known and available in the art. Exemplary methods are described in, for example, U.S. Publication Application No. 2010/0120098, which is incorporated herein by reference in its entirety. This technology is illustrated in FIG. 1, which is also provided by the commercially available Illumina Nextera platform.

As shown in FIG. 1, target polynucleotide 101 is incubated with transposomes 103 and 105 (also referred to as transposition complexes). Each transposition complex can include a transposase and DNA oligonucleotides that exhibit the nucleotide sequences of a transposon, including the transferred transposon sequence and its complement (i.e., the non-transferred transposon end sequences) as well as other components to form a functional transposome or transposition complex. See, e.g., US Patent Application Publication No. 2010/0120098. The DNA oligonucleotides can further comprise additional sequences (e.g., primer binding sequences) as desired. The DNA oligonucleotides that exhibit the nucleotide sequences of a transposon and those DNA oligonucleotides that further comprise additional sequences (e.g., primer binding sites, restriction sites, etc.) are collectively referred to as transposon end sequences. As shown in FIG. 1, the transposition complex 103 includes transposon end sequences 109 and transposase 107, and the transposition complex 105 includes transposon end sequences 111 and transposase 107.

Step (a) of FIG. 1 illustrates a tagmentation reaction. Tagmentation is similar to transposon insertion, except a transposition complex cuts the target polynucleotide and appends or tags transposition end sequences to the resulting polynucleotide fragments. Thus, during tagmentation, the transposition complexes 103 and 105 bind to the target polynucleotide 101 and simultaneously fragment and tag the target polynucleotide, adding transposon end sequences 109 and 111 to the fragmented target polynucleotide, thereby generating tagged polynucleotide fragment 113. Then, transposases are removed from the tagged polynucleotide fragment 113 in step (b).

The previous tagmentation step leaves a short single stranded sequence gap in the tagged polynucleotide fragments. As shown in step (c), fragmented ends of the tagged polynucleotide fragment 113 are repaired and extended with a strand-displacing DNA polymerase. These extended fragments are also referred to as the tagged polynucleotide fragments in embodiments of the present invention. As shown in step (d), limited-cycle PCR can be performed with four primers: a terminal primer 114, a barcoded adapter primer 115, a terminal primer 116, and a barcoded adapter primer 117. This limited-cycle PCR reaction adds the barcoded adapters 125 and 127 to the tagged polynucleotide fragment 113.

As shown in FIG. 1, each of the barcoded adapter primers 115 and 117 comprises three regions. The barcoded adapter primer 115 comprises a transposon end sequence 115a, a barcode sequence 115b, and a support sequence 115c. The barcoded adapter primer 117 comprises a transposon end sequence 117a, a barcode sequence 117b, and a support sequence 117c. As shown in FIG. 1, the barcoded adapter primers are capable of hybridizing to the transposon end sequences located at terminal ends of the tagged polynucleotide fragment 113. The support sequences 115c and 117c comprise sequences that can either hybridize or are complementary to capture oligonucleotides immobilized on the surface of a sequencing support (e.g., a flow cell). A unique set of barcoding sequences 115b and 117b is incorporated into polynucleotide fragments during PCR, allowing them to be distinguishable from other polynucleotide fragments comprising a different set of barcoding sequences. However, transposon end sequences (115a and 117a) and support sequences (115c and 117c) may be universal for all samples. In other words, unlike barcoding sequences, the conserved regions (e.g., transposon end sequences and support sequences) of adapter primers used for a plurality of samples may have the same nucleotide sequences.

The terms, i5 and i7, shown in FIG. 1 are nomenclatures used in the Illumina sequencing platform. In the Illumina platform, the terminal primer 114 and the terminal primer 116 are referred to as i5 and i7 terminal primers, respectively, and the barcoded adapter primer 115 and the barcoded adapter primer 117 are referred to as i5 index primer and i7 index primer, respectively. In Illumina MiSeq instrument, the i7 index is adjacent to the P7 sequence (i.e., capture oligonucleotide), and the i5 index is adjacent to the P5 sequence (i.e., capture oligonucleotide) on the sequencing support (e.g., flow cell).

The primers in the Illumina Nextera sample preparation kit have the following sequences:

i5 terminal primer 116:
(SEQ ID NO: 193)
5′-AATGATACGGCGACCACCGA
i7 terminal primer 118:
(SEQ ID NO: 194)
5′-CAAGCAGAAGACGGCATACGA
i5 index primer (barcoded adapter primer 115):
(SEQ ID NO: 195)
5′ AATGATACGGCGACCACCGAGATCTACAC[i5]TCGTCGGCAGCGTC
i7 index primer (barcoded adapter primer 117):
(SEQ ID NO: 196)
5′ CAAGCAGAAGACGGCATACGAGAT[i7]GTCTCGTGGGCTCGG

In the i5 and i7 index primers shown above, the positions of the barcode sequences are shown as [i5] and [i7], respectively. As shown in FIG. 1, the barcode positions [i5] and [i7] are noted as “NNNNNNNN” in FIG. 1, where each “N” is equivalent to one unknown nucleotide for the barcode sequences.

After PCR amplification in step (e), barcoded polynucleotide fragments 123 are generated. As shown in FIG. 1, the barcoded polynucleotide fragment 123 is flanked by a set of barcoded adapters 125 and 127. Each of the barcoded adapters 125 and 127 includes three regions of sequences as the barcoded adapter primers 115 and 117, respectively. After the PCR reaction, polynucleotide fragments having a small size are removed from the resulting PCR products in step (f).

In the flowchart illustrated in FIG. 1, primer sequences, transposases, sequencing platforms, and other specific components discussed above are merely exemplary. One of ordinary skill in the art would recognize many variations, modifications, and alternatives in generating a library of sequence-ready, barcoded DNA fragments.

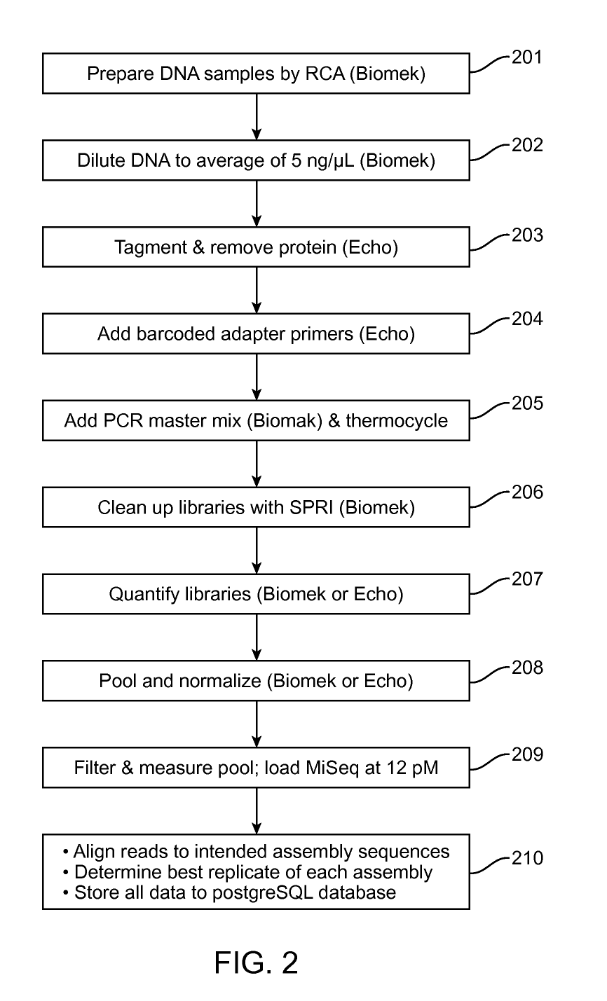

FIG. 2 is a high level flowchart illustrating a method of preparing and simultaneously sequencing a plurality of DNA samples (e.g., input polynucleotides) according to an embodiment of the present invention. While the flowchart shown in FIG. 2 incorporates some of the steps shown in FIG. 1, there are several differences and advantages of the embodiment illustrated in FIG. 2. First, as described above, compositions and method provided herein are capable of highly multiplexed sequencing of a greater number of samples (e.g., over 4000 samples) as compared to commercially available kits which are commonly limited to preparing and simultaneously sequencing up to only 96 samples. Highly multiplexed sequencing is enabled in methods and compositions provided herein, partly due to hundreds of novel barcode sequences generated by the present method, which allow thousands of DNA samples to be tagged and resolved during sequencing. Second, the tagmentation reaction volumes have been reduced by several orders of magnitude as compared to commercial kits (e.g., 100-fold less), thereby reducing cost and increasing efficiency of the sequencing process. Third, many commercially available kits require pure input DNA for tagmentation, an accurate assessment of its concentration, and a column clean-up that are labor intensive and cost prohibitive for high-throughput sample preparation. To overcome these problems, as shown in the exemplary workflow shown in FIG. 2, the sample preparation has been simplified. For example, in some embodiments, samples are prepared by rolling circle amplification, which simplifies the DNA quantitation and dilution process prior to tagmentation. In another example, transposases can be deactivated after tagmentation without using column cleanup or other solid phase extraction methods (e.g., binding matrix beads) to remove transposases. One or more combinations of these features can increase efficiency of the overall sample preparation and sequencing process. These features and other advantages of the compositions and methods provided herein are further described in detail with reference to FIG. 2.

In the exemplary workflow shown in FIG. 2, one or more process steps are optimized for sequencing a large number of samples per sequencing run. For all samples to achieve similar average coverage and threshold coverage (e.g., 15×) during sequencing, it is desirable that each sample in the pool has a similar molar concentration of sequenceable fragments. To pool according to molar concentration, it is desirable that the average fragment size of thousands of samples is determined in a reliable manner, which can be time-consuming and labor-intensive. One or more process steps shown in FIG. 2 contribute in minimizing the variation in average polynucleotide fragment size across the libraries so that pooling in step (208) can be based on a mass concentration of polynucleotides for each sample. In other words, the pooling of libraries in step (208) can be achieved without determining the distribution of fragment sizes for every library, which can be time-consuming for a high throughput operation. In certain embodiments, the libraries of sequenceable fragments from different libraries can be pooled together in step (208) without quantifying the libraries in step (207) or normalizing the libraries in step (208).

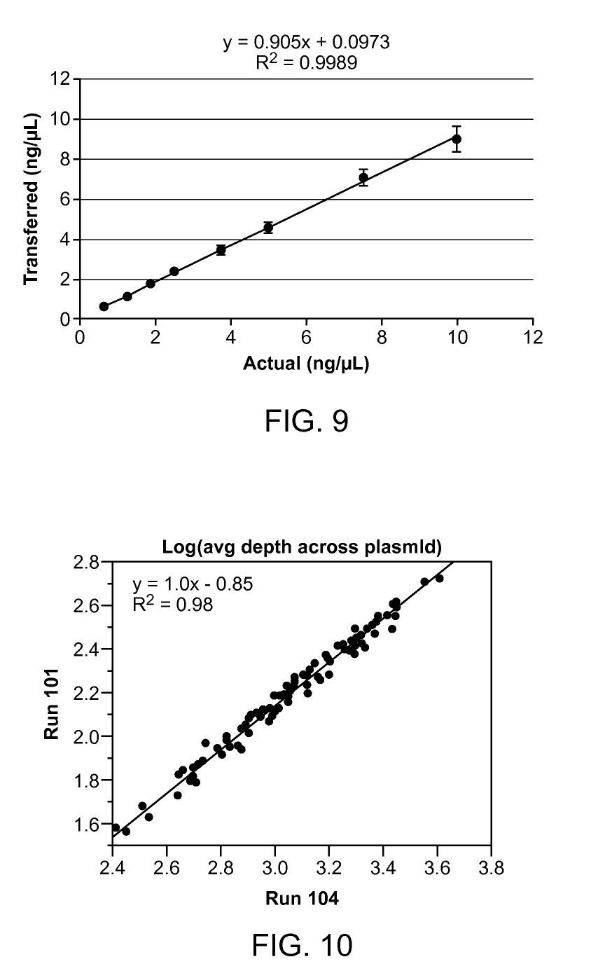

In the exemplary embodiment shown in FIG. 2, some of the steps in the flowchart require transferring a very small volume of liquid (e.g., less than 2 μL). Such steps may be performed by an acoustic liquid transfer system such as an Echo 550 plus Access robotics (Labcyte, Sunnyvale, Calif.). For transferring a larger volume of liquid (e.g., 2 μL or greater), a manual or robotic liquid handling system, such as Biomet FX or NX robots, may be used. In transferring certain range of volumes (e.g., 2 μL to 50 μL), either type of liquid transfer devices may be used. When handling a solution containing high molecular weight polynucleotides (e.g., RCA polynucleotides having a concentration greater than 10 ng/μL), a conventional liquid handler, such as Biomek, may be used instead of an acoustic liquid transfer system. See, e.g., step (202) of FIG. 2. It was found that an acoustic liquid transfer system can reliably transfer solutions comprising polynucleotides at concentrations of 10 ng/μL or less. See, e.g., FIG. 9. It is noted that the liquid transfer devices indicated in the parentheses in FIG. 2 are merely exemplary, and other suitable liquid transfer devices may be used.

Referring to FIG. 2, in one embodiment, the input polynucleotide from a sample can be prepared by rolling circle amplification (201). Rolling circle amplification is an isothermal process for generating multiple copies of a sequence, and it can be adopted in vitro for DNA amplification. See, e.g., Fire et al., Proc. Natl. Acad. Sci. USA, 1995, 92:4641-4645; Lui et al., J. Am. Chem. Soc. 1996, 118:15897-1594; U.S. Pat. No. 7,714,320. In some embodiments, commercially available kits, such as Illustra Templiphi kit (GE Healthcare Life Sciences, Piscataway, N.J.), may be used for rolling circle amplification of a DNA sample. In an embodiment, a DNA sample may include a plasmid DNA which can be replicated and amplified in an RCA solution comprising a suitable DNA polymerase (e.g., phi29) and other reagents to generate a target polynucleotide. For all samples, the RCA reaction is generally performed in an equal volume of the same RCA solution so that an approximately same amount of target polynucleotides can be generated for each of the samples.

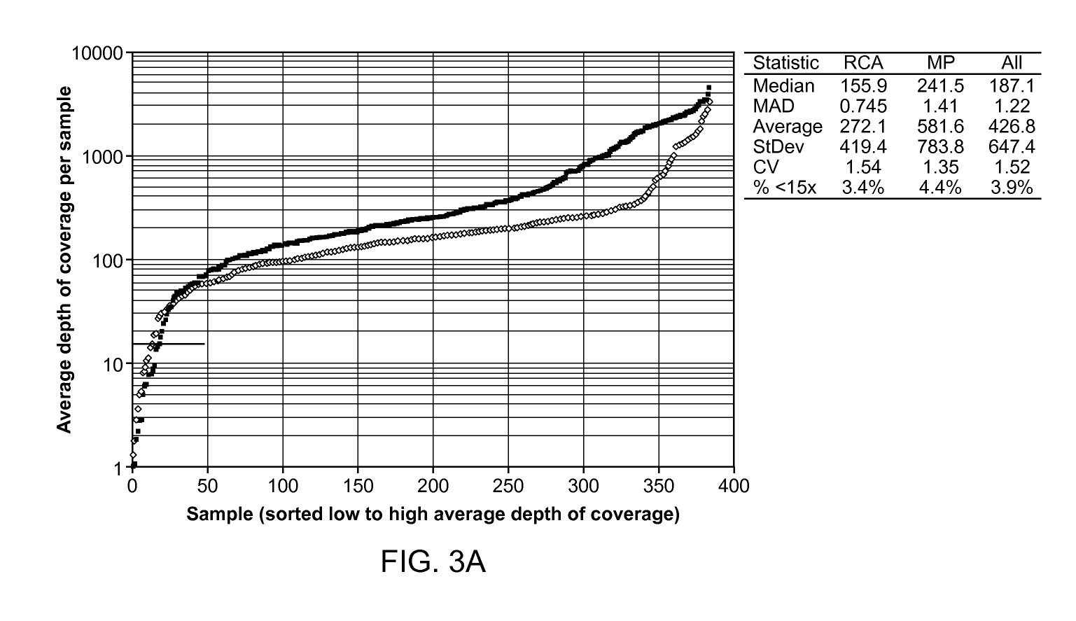

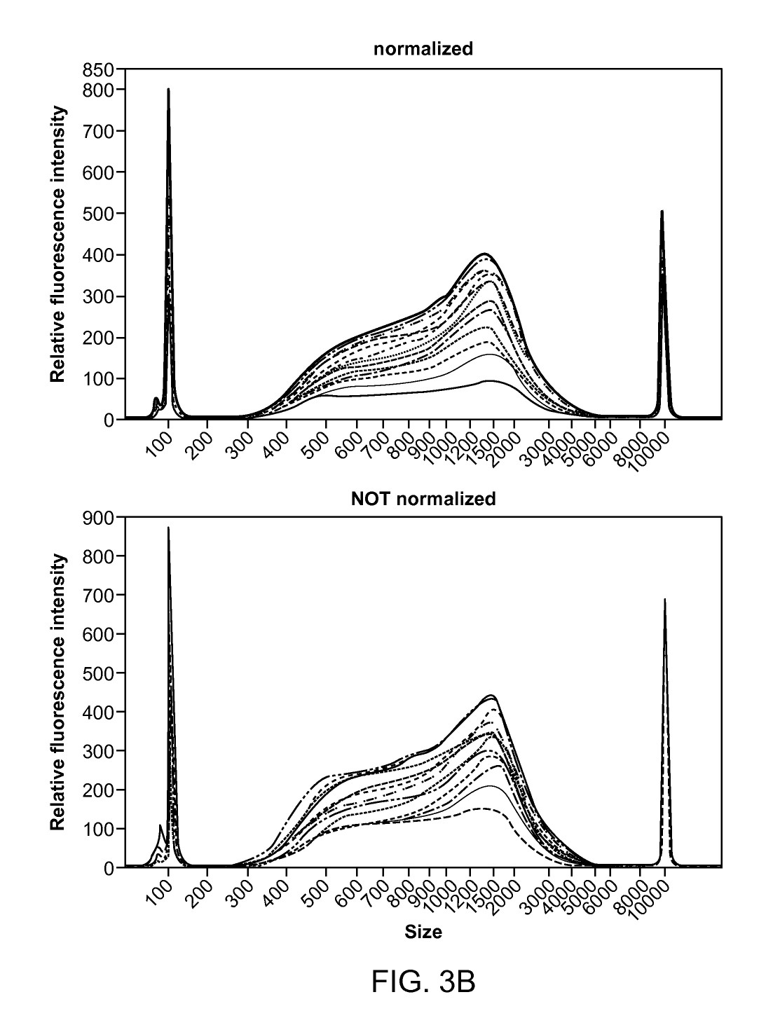

When RCA prepared target polynucleotides are used in tagmentation reactions, it was discovered by the present inventors that the size distributions of RCA prepared target polynucleotides that had been normalized before tagmentation were very similar to those that had not been normalized. See, e.g., FIG. 3B. It was also discovered that the pre-tagmentation normalization did not appear to affect the overall variation in depth of sequencing coverage across many samples (results not shown). These results suggest that target polynucleotides amplified by RCA is of even concentration across many samples, and that RCA prepared target polynucleotides can be used for tagmentation without normalizing each individual sample.

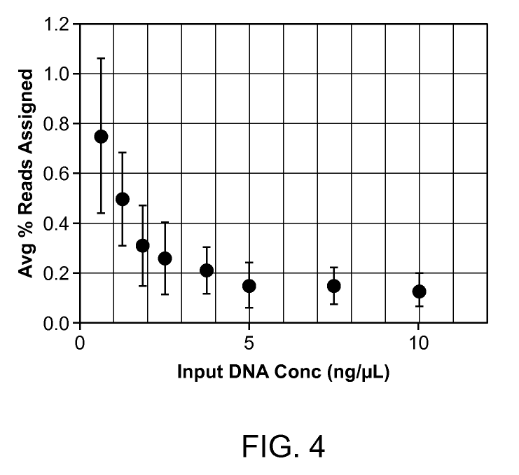

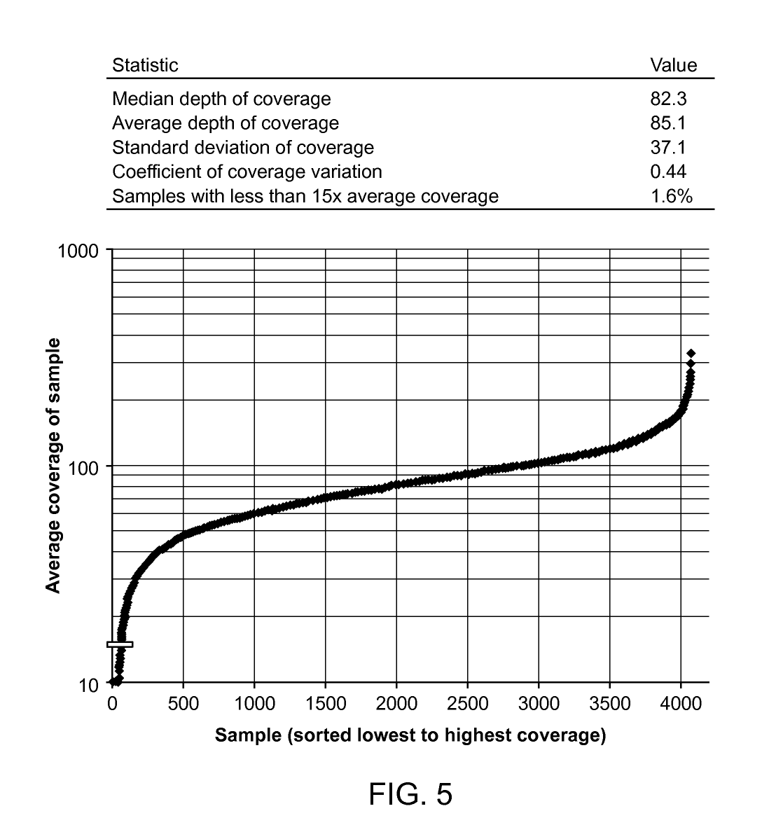

It was also discovered by the present inventors that when the polynucleotide concentration in the RCA solution is diluted to about 3 ng/μL to about 10 ng/μL (e.g., average of about 5 ng/μL) prior to the tagmentation step, then the quality of sequencing improves for pooled samples. See, e.g., FIG. 4. More specifically, if the target polynucleotide concentration for all samples is between about 3 ng/μL to about 10 ng/μL prior to their transfer for tagmentation reactions, then the resulting polynucleotide fragments render relatively consistent sequencing coverage and less coverage variability across all samples as shown in FIG. 5.

Referring to step (202) of FIG. 2, each RCA solution comprising a target polynucleotide can be diluted by a standard dilution factor (i.e., same for all samples), prior to the next tagmentation step, since RCA produces a relatively consistent final concentration of target polynucleotides across all samples. A standard dilution factor of 1 to 12 may be used in certain embodiments (see, e.g., Examples section) to dilute RCA solutions across all samples because it was empirically determined that this standard dilution factor provides a target polynucleotide concentration of about 5 ng/μL on average for all samples. Once a suitable standard dilution factor is empirically determined for a set of experimental conditions, the standard dilution factor may be used to dilute all RCA solutions without quantifying target polynucleotides and diluting each sample individually. The dilution of RCA solutions by a standard dilution factor can lead to a significant amount of savings in terms of time and cost.

A suitable standard dilution factor may be determined in a number of different ways. In one embodiment, a standard dilution factor may be determined by quantifying target polynucleotides in at least a portion of a plurality of RCA solutions. For example, if there are 4000 RCA solutions comprising target polynucleotides, then the polynucleotide concentration may be quantified for each of 4000 RCA solutions. In some embodiments, the polynucleotide concentration in a portion of the samples (e.g., a single 384-well plate instead of all plates) may be measured since RCA provides a relatively consistent final concentration of target polynucleotides. Based on the measured concentration of target polynucleotide in each RCA solution, an average concentration of target polynucleotides in all or at least a portion of RCA solutions may be calculated. The standard dilution factor to dilute each RCA solution can then be determined by dividing the average concentration by any number selected from 3 ng/μL to 10 ng/μL, as this range was found to provide relatively consistent sequencing coverage and less variability during sequencing. In an embodiment, a number in the middle of the range (e.g., 5, 6, or 7 ng/μL) can be selected for determining a standard dilution factor. In an embodiment, the standard dilution factor is calculated by dividing the average concentration by 5 ng/μL. Thus, in certain embodiments, an average of about 1.5 ng to about 5 ng of polynucleotides is used in a tagmentation reaction volume of 0.5 μL. In another embodiment, an average of about 3 ng to about 10 ng of polynucleotides is used in a tagmentation reaction volume of 1 μL. In another embodiment, an average of 6 ng to 20 ng of polynucleotides is used in a tagmentation reaction volume of 2 μL.

In another embodiment, a standard dilution factor may be determined by measuring a concentration of target polynucleotides in a mixed RCA solution. For example, an equal volume of RCA solutions derived from all samples (or at least a portion thereof) can be mixed together, thereby generating a mixed RCA solution comprising target polynucleotides. Thereafter, an average concentration of target polynucleotides in the mixed RCA solution can be determined. This requires quantification of only a single “mixed” RCA solution. Based on the concentration of polynucleotides in the mixed RCA solution, a suitable standard dilution factor may be determined.

In step (202), any suitable methods can be used to quantify a concentration of polynucleotides in a solution. For example, a fluorescent dye, PicoGreen dsDNA quantitation reagent (Quant-iT PicoGreen dsDNA assay kit, Life Technologies, Foster City), may be used. The method utilizes the increased fluorescent intensity that is observed when PicoGreen binds to dsDNA. The fluorescent intensity of the PicoGreen dye is measured with a spectrofluorometer capable of producing the excitation wavelength of about 480 nm and recording at the emission wavelength of about 520 nm.

While steps (201) and (202) in FIG. 2 illustrate preparing samples by RCA, embodiments of the present invention are not limited to using RCA for sample preparation. Other suitable sample preparation methods such as plasmid mini-preparation or PCR amplicons may be used if desired. In some embodiments, if desired, each individual sample may be quantified and/or diluted based on the individually measured DNA concentration prior to the tagmentation step so that the dilution may be adjusted as necessary.

Referring to FIG. 2, the diluted DNA sample can be fragmented and tagged in a tagmentation reaction with transposomes or transposition complexes, and subsequently, transposases can be removed from the tagged DNA fragments (203). As described in relation to FIG. 1, target polynucleotides can be incubated with transposases pre-loaded with transposon end sequences to fragment and tag the target polynucleotides with transposon end sequences. The method for inserting transposon end sequences into the target polynucleotides can be carried out in vitro.

Any suitable transposomes or transposition complexes may be used in the present method. Some of them are known in the art and available as commercially available kits. For example, the Ez-Tn™ hyperactive Tn5 Transposase and the HyperMu™ Hyperactive MuA Transposase are available from Epicentre Technologies, Madison, Wis. See, also, U.S. Patent Application Publication No. 2010/0120098, which is incorporated herein by reference in its entirety. In an embodiment, the transposition complexes may include transposases such as Tn5 or MuA and their respective transposon terminal end sequences. See, e.g., Goryshin and Reznikoff, J. Biol. Chem., 237: 7367, 1998; and Mizuuchi, Cell, 35: 785, 1983; Savilahti et al., EMBO J., 14: 4893, 1995; which are incorporated by reference in their entireties. Other transposition complexes including transposases, such as Tn552, Ty1, Tn7, and Tn3, may be used in some embodiments of the present invention. Transposomes or transposition complexes are also commercially available as kits and can be purchased from, for example, Illumina Inc. (Nextera DNA library preparation kit), KAPA Biosystems (Kapa DNA library preparation kits), Molecular Cloning Laboratories (Next DNA sample kit), New England Laboratory (NEB Next kits), and the like.

A suitable ratio of transposomes to target polynucleotides for tagmentation reaction can be determined based on knowledge in the art and the present disclosure. Generally, it is desirable to have a relatively precise transposomes to target polynucleotide ratio during tagmentation. The ratio can affect the quality of tagmentation as well as coverage during sequencing. The extent of the fragmentation and/or the size of fragments can be controlled using appropriate reaction conditions such as by using the suitable concentration of transposomes and controlling the temperature and time of incubation. In an embodiment, suitable reaction conditions can be obtained using known amounts of a test library of nucleic acids and titrating the transposomes and time to build a standard curve for actual sample libraries. Exemplary tagmentation reaction conditions are also described in detail in the Examples section.

In an embodiment, any suitable tagmentation reaction volumes may be selected to fragment and tag target polynucleotides. In some embodiments, a suitable tagmentation reaction volume may include 10, 9, 8, 7, 6, 5, 4, 3, 2, 1, 0.5, 0.1, 0.01, 0.005 μL or any number in between these numbers. For highly multiplexed sequencing, tagmentation reactions are generally performed in a small volume. A small tagmentation volume requires a reduced amount of transposases and other tagmentation reagents, which can save cost. Furthermore, if an acoustic liquid transfer system (e.g., Echo 550, Labcyte, Sunnyvale, Calif.) is used, it does not require pipettes for liquid transfer, reducing potential contamination between samples. In some embodiments, a suitable tagmentation reaction volume may include between about 0.005 μL to about 2 μL. In certain embodiments, the tagmentation reaction is performed at a volume of about 2 μL or less, typically about 1 μL or less, and more typically at about 0.5 μL. For a small reaction volume of 0.5 μL, typically 200 nL of DNA (having a concentration between about 3 ng/uL to about 10 ng/uL, typically about 5 ng/μL) can be added to 300 nL of a tagmentation enzyme solution which includes transposition complexes and reagents. In other words, about 0.6 ng to about 2 ng (typically about 1 ng) of target polynucleotide is generally used in a tagmentation reaction having a volume of about 0.5 μL.

In some embodiments as shown in the Examples section, the tagmentation reaction is performed at 0.5 μL, which is 100-fold less than the tagmentation reaction volume required in the Illumina Nextera kit. It was discovered by the present inventors that the 100-fold reduction in tagmentation volume does not change the quality of sequencing coverage or variability. For example, as shown in FIG. 5, when more than 4000 samples are prepared at a tagmentation volume of 0.5 μL, less than 2% of samples had less than 15× average coverage. In an embodiment, the 15× coverage can be set as a threshold as part of quality control to determine the rate of sample loss. For example, in FIG. 5, the rate of sample loss for over 4000 samples is only 1.6%.

Referring to FIG. 2, transposases bound to the tagged polynucleotide fragments can be removed using any suitable removal methods so that the enzymes do not interfere with the subsequent PCR reaction (203). In certain embodiments, the transposases may be removed without column spins, other solid phase extraction methods (e.g., using DNA binding matrix beads), or centrifugation. These physical separation means are typically required in some tagmentation kits, which can be labor intensive and costly for high-throughput process. In an embodiment, the transposases may be removed under a dissociation condition, such as application of heat to dissociate transposases or the addition of a dissociation solution. For example, a dissociation solution, when added to the tagmentation reaction mixture, may change the ionic strength of the resulting tagmentation reaction solution and promote removal of transposases from tagged polynucleotide fragments. In some embodiments, the dissociation solution may include a detergent, a denaturing salt, a high pH, or any combination thereof. After dissociating and removing transposases from the tagged polynucleotide fragments, adapter primers can be added directly to the tagmentation reaction mixture. The present transposase removal methods can save a significant amount of time and cost for high-throughput process.

In an embodiment, a dissociation solution may comprise an ionic surfactant, such as sodium dodecyl sulfate (SDS). For example, a dissociation solution comprising SDS at a final concentration of about 0.05% to about 0.3%, more typically about 0.1% (weight per volume percent) may be used to remove transposases. The final concentration of SDS may refer to the concentration of SDS when the solution comprising SDS is added to a tagmentation reaction mixture (containing tagged polynucleotide fragments, transposases, and other components used in the tagmentation reaction). For example, 125 nL of 0.5% SDS in TE can be added to 500 nL of the tagmentation mixture, which results in a final SDS concentration of 0.1%. In some embodiments, the dissociation solution consists of SDS as a dissociation or denaturing agent in TE (or other suitable buffers). In some embodiments, other dissociation agents may be used alone or in combination with SDS. For example, Triton X-100 may be used in combination with SDS. In some embodiments, a dissociation solution may comprise 1% Triton X-100 and 0.3% SDS.

While there are advantages to using a dissociation condition without column spins or other solid phase extraction, embodiments of the present invention are not limited to using specific transposase removal methods. Any suitable removal methods, column spin or DNA binding matrix beads, may be used to separate transposases from polynucleotide fragments prior to PCR. For example, commercially available kits, such as Zymo kit (Illumina, San Diego, Calif.), may be used.

Referring to FIG. 2, the adapter primers may be added to the tagged DNA fragments generated by the tagmentation reaction (204). The adapter primers are capable of hybridizing to the tagged polynucleotide fragments generated in step (203) and generating barcoded polynucleotide fragments. As shown in FIG. 1, an adapter primer may include one or more universal sequences that are commonly used for all samples, and a barcode sequence which is unique to each sample and its input polynucleotide. For example, one or more universal sequences in the adapter primer may include a transposon end sequence (e.g., 115a and 117a shown in FIG. 1) that is complementary to the 3′ ends of each of the sense and/or anti-sense strand of a tagged polynucleotide fragment. The one or more universal sequences in the adapter primer may also include support sequences (e.g., 115c and 117c shown in FIG. 1), which can later be used to anchor the barcoded polynucleotide fragments onto the surface of a sequencing support (e.g., a flow cell). In an embodiment, adapter primer sequences may be selected based on the transposon tags (e.g., transposon end sequences) incorporated into tagged polynucleotide fragments. The support sequences in the adapter primers may also be selected based on capture oligonucleotides present on the sequencing support surface. Furthermore, an adapter primer may be any suitable length as long as it can introduce a barcode sequence and other functional sequences (e.g., a terminal primer binding site, sequencing primers, etc.) to the tagged polynucleotide fragments.

In an embodiment, the barcode sequence can be a sequence of synthetic nucleotides or natural nucleotides that allow for easy identification of the polynucleotide fragments to which it is attached in a collection of other polynucleotide fragments. Generally, barcode sequences are of sufficient length and comprise sequences that are sufficiently different from one another. For example, each barcode sequence may include at least 4, 5, 6, 7, 8, 9, 10, 11, 12, 13, 14, 15, 16, or more nucleotides in length. In an embodiment, a barcode sequence may include 8 nucleotides in length. The barcode sequences generated by the present method (see section 6.3 below) can be used to uniquely tag polynucleotide fragments from each sample (i.e., input polynucleotide). In some embodiments, the barcode sequences designed according to the present method can be incorporated into any suitable adapter primers. For example, the present barcode sequences can be incorporated into Illumina i5 and i7 index primers if the Illumina MiSeq or other sequence platform is used for sequencing. In this embodiment, any one of barcode sequences SEQ ID NO: 1 through 192 may be inserted into positions [i5] and [i7] of adapter primers having SEQ ID NO: 195 and SEQ ID NO: 196, respectively.

In an embodiment, a pair of unique barcode sequences may be introduced to each polynucleotide fragment. After introducing a pair of barcode sequences into polynucleotide fragments and dually indexing them, a suitable sequencing instrument can be used to read both barcode sequences to identify the source of the polynucleotide fragments (e.g., input polynucleotide from a sample). Through dual indexing, sample misidentification inaccuracies can be reduced. For sequencing a smaller number of samples, however, a single barcode sequence may be used if desired.

In step (204) of FIG. 2, any suitable amount of adapter primers can be added to the tagmentation reaction solution generated in step (203). For example, to a tagmentation reaction solution having a volume of 625 nL generated in step (203), 125 nL of each of the adapter primer pairs (at e.g., 100 μM) may be added. See the Examples section for details. The amount or volumes of adapter primers can be readily determined and adjusted by those skilled in the art. While FIG. 2 illustrates adding adapter primers in step (204), which is separate from PCR step (205), all PCR reagents and adapter primers may be added concurrently in step (205).

The PCR reaction can be initiated in a reaction chamber comprising a PCR master mix and a tagmentation reaction solution that includes tagged polynucleotides and adapter primers under a suitable thermocycling condition (205). A PCR master mix may include a solution that contains water, 10× Thermopol buffer, MgSO₄, DNA polymerase, dNTPs, MgCl₂, deoxynucleotide triphosphates, terminal primers, and a DNA polymerase at their optimal concentrations for efficient amplification of template DNA by PCR. As shown in FIG. 1, the adapter primers can hybridize to the tagged polynucleotide fragments to generate barcoded polynucleotide fragments, and the terminal primers can hybridize to terminal ends of barcoded polynucleotide fragments as templates to further amplify these fragments. In an embodiment, the components of the PCR master mix may be added concurrently. In another embodiment, the components may be added at different times before PCR. Additional details of an exemplary PCR master mix and thermocycling conditions are further described in the Examples section.

In an embodiment, the PCR master mix may include a large amount of water or other suitable aqueous solution to dilute the tagmentation reaction solution generated in the previous step (203). The large dilution prevents transposases in the solution from interfering with the PCR reaction. For example, if the tagmentation reaction is performed at a volume of 0.5 μL, then 20.275 μL of water may be added together with other PCR reagents to bring the final volume of PCR reaction to 25 μL. While this exemplary dilution illustrates a 50-fold dilution of the tagmentation mixture (i.e., 0.5 μL diluted to 25 μL), any suitable dilution ratio may be used to prevent transposases from interfering with PCR. For example, the tagmentation mixture may be diluted by at least 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, or more. The reduced amount of template polynucleotide during PCR can be compensated by adjusting the number of PCR cycles. In an embodiment, 8 to 24 cycles of PCR, more typically about 12 cycles, may be used to generate and amplify barcoded polynucleotide fragments.

While FIG. 2 illustrates an embodiment where adapters or barcode sequences are introduced into polynucleotide fragments using tagmentation and PCR, embodiments of the present invention are not limited to using these reactions for appending adapters and/or barcode sequences. As described above, the adapters and/or barcode sequences may be attached to polynucleotide fragments using any suitable techniques known in the art. For example, blunt end ligation methods may be used to introduce these sequences into polynucleotide fragments.

Referring to FIG. 2, to control the size distribution of polynucleotide fragments, the libraries of PCR products can be cleaned to remove unincorporated primers and small fragments (206). Any suitable cleaning methods, such as solid reverse immobilization (SPRI) beads, may be used to remove undesired fragments and primers. In an embodiment, SPRI beads (e.g., Ampure XP paramagnetic beads) can be added to PCR products at any suitable volume ratio (e.g., 0.6 to 1). By selecting suitable SPRI beads and volume ratios, the fragments having a size greater than 300 base pairs may be selected.

In some embodiments, a “double-sided” solid reverse immobilization (DSPRI) purification protocol can be used to clean the libraries of PCR products. Polynucleotide fragments that have a high proportion of larger fragments (e.g., greater than 1000 base pairs) can result in a lower average depth coverage during sequencing. During the DSPRI, a first set of beads may be added to the polynucleotide fragments at a low volume to remove large fragments (e.g., greater than 1000 base pairs), and the supernatant is then collected. A second set of beads can then be added to the supernatant to remove small fragments (e.g., less than 300 base pairs). The DSPRI protocol may enrich DNA fragments having a length between 300 and 800 base pairs, which is desirable for next-generation sequencing. By removing populations of both small fragments and large fragments prior to sequencing, the average depth of sequencing may be improved.

After cleaning the libraries of barcoded polynucleotide fragments by removing undesired fragment sizes, the polynucleotide fragments in the libraries can be quantified if desired (207). To achieve the highest quality of data on sequencing platforms, the barcoded polynucleotide fragments from each sample can be accurately quantified so that they can be combined at equal molar ratios with barcoded polynucleotide fragments from other samples. This process can improve even depth of coverage across the combined pool of polynucleotide fragments. The DNA quantification of libraries can be performed using any suitable methods, such as PicoGreen assay. The details of an exemplary protocol for the PicoGreen assay are further described in the Examples section. In some embodiments, other dsDNA-specific fluorescent dye method, such as Qubit, may be used to quantify the library.

Each of steps (201) through (207) shown in FIG. 2 can be repeated for the plurality of input polynucleotides derived from different samples to generate libraries of barcoded polynucleotide fragments. Thus, each library has barcoded polynucleotide fragments that are tagged with one or more barcode sequences that are unique to each library. If the barcoded polynucleotide fragments are tagged with a pair of barcode sequences, then different combinations of the barcode sequences can be used to distinguish polynucleotide fragments derived from different sources or samples (e.g., input polynucleotides).

Referring to FIG. 2, in certain embodiments, the libraries of barcoded polynucleotide fragments can be normalized and pooled together prior to sequencing (208). In an embodiment, the volume of each library to combine into a pool for sequencing is determined based on the library quantification in step (207), assuming that the average fragment size of the library is 500 base pairs, and normalizing for the input polynucleotide length (e.g., plasmid length). It was empirically determined that the average fragment size of each library at this stage prior to pooling is about 500 base pairs. It is believed that the prior steps of the workflow shown in FIG. 2 (e.g., dilution of polynucleotides, tagmentation reaction, transposase deactivation, PCR reactions, cleaning up libraries with SPRI beads, and the like) result in a relatively uniform polynucleotide fragment size at this stage. Thus, instead of measuring the average fragment size of thousands of samples using a Bioanalyzer, which is time-consuming and labor-intensive, the molar concentration of each library can be calculated assuming that the average fragment size of each library is 500 base pairs.

Furthermore, in step (207), the libraries can be normalized for the input polynucleotide length prior to pooling in certain embodiments. As an illustration, if all the libraries are derived from a plasmid having the same length, then all the libraries are pooled together at an equal volume (assuming that the libraries have the same concentration of DNA). On the other hand, if the first library is derived from a plasmid which has twice the length as the second library, then the volume of the first library added into a pool will be twice as large as the second library (assuming that both libraries have the same DNA concentration). This way, the entire length of both plasmids will be equally presented to a sequencer for even coverage of all the libraries.

While steps (207) and (208) can improve the depth of sequencing coverage across the combined pool of polynucleotide fragments, these steps are optional and can be omitted for expediency without greatly reducing the quality of sequence data.

Referring to FIG. 2, in some embodiments, the pool of combined libraries of barcoded polynucleotide fragments can be filtered and concentrated using a filter to remove small fragments having a size less than 300 base pairs (209). This additional filtering process can improve sequencing coverage for the majority of barcoded polynucleotide fragments. Any suitable filters may be used for removing small fragments. Exemplary filters include a Microcon Fast-Flow filter unit (EMD Millipore, Billerica, Mass.).

In certain embodiments, the filtered pool of polynucleotide fragments can then be further characterized before sequencing in step (209). For example, the distribution of fragment sizes of the pooled polynucleotide fragments can be measured using a Bioanalyzer, Fragment Analyzer, or by integrating the signal intensity along an agarose gel. The molar concentration of the pooled DNA sample can be calculated using PicoGreen value and the measured average fragment size as further described in the Examples section. For example, the molar concentration of the pooled polynucleotide fragments can be calculated as follows:

Molar concentration (nM)=PicoGreen value (ng/μL)×1,000,000/(660×avg fragment size)

Any suitable sequencer (e.g., MiSeq) can be used to load a combined pool of barcoded polynucleotide fragments at a suitable molar concentration (e.g., 12 pM) as recommended by the sequencer. The sequence reads generated from the sequencer can be sorted or demultiplexed based on the barcode sequences using the software provided with the sequencer.

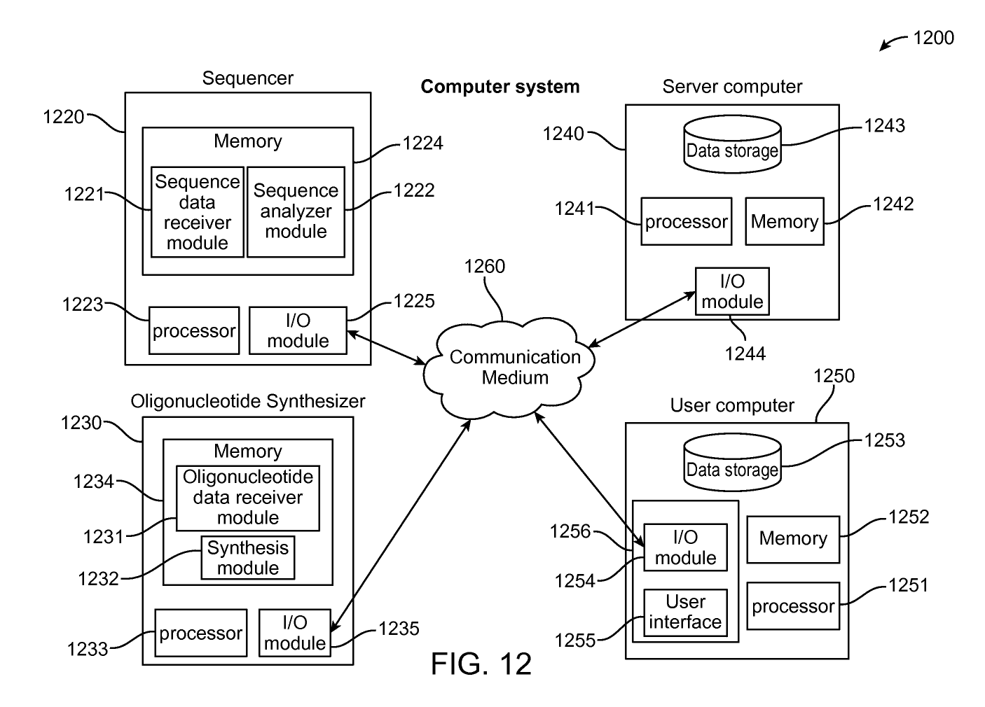

The workflow shown in FIG. 2 can further include aligning sequence reads generated from the sequencer to its corresponding reference sequence (e.g., the intended assembly sequences in the plasmid) (210). For samples containing DNA assemblies stitched from several DNA components, it may be desirable to sequence replicates (e.g., multiple clones) as part of quality control. In these embodiments, the sequence reads from each replicate can be compared against its reference sequence stored in a database. The aligned sequences for each replicate can then be compared, and the best replicate (e.g., with read sequences with no deletions, mutations, or substitutions compared to the reference sequence) may be determined. All data generated by the sequence reads can then be stored in any suitable data storage, such as those exemplified in the computer system of FIG. 12.

It should be appreciated that the specific steps illustrated in FIG. 2 provide particular methods of generating and/or sequencing a plurality of polynucleotides according to an embodiment of the present invention. Other sequences of steps may also be performed according to alternative embodiments. For example, alternative embodiments of the present invention may perform the steps outlined above as multiple sub-steps as appropriate to the individual step. Furthermore, additional steps may be added or removed depending on the particular applications. Additionally, the features described in other figures or parts of the application may be combined with the features described in FIG. 2. One of ordinary skill in the art would recognize many variations, modifications, and alternatives.

### Generating Barcode Sequences and Synthesizing Oligonucleotides

In another aspect, provided herein are barcode sequences, adapter primers comprising barcode sequences, and methods of generating these sequences suitable for highly multiplexed sequencing. In some embodiments, unique barcode sequences can be incorporated into adapters, which are appended to polynucleotide fragments to generate barcoded polynucleotide fragments for sequencing. In some embodiments, unique barcode sequences may be appended or ligated directly to the tagged polynucleotide fragments. The specific sequence or “index” used as a barcode sequence is unrestricted. It can be any suitable length, such as 6, 7, 8, 9, 10, 11, 12, or the like. Generally, barcode sequences are of sufficient length and comprise sequences that are sufficiently different from other barcode sequences to allow the identification of samples to which they are associated.

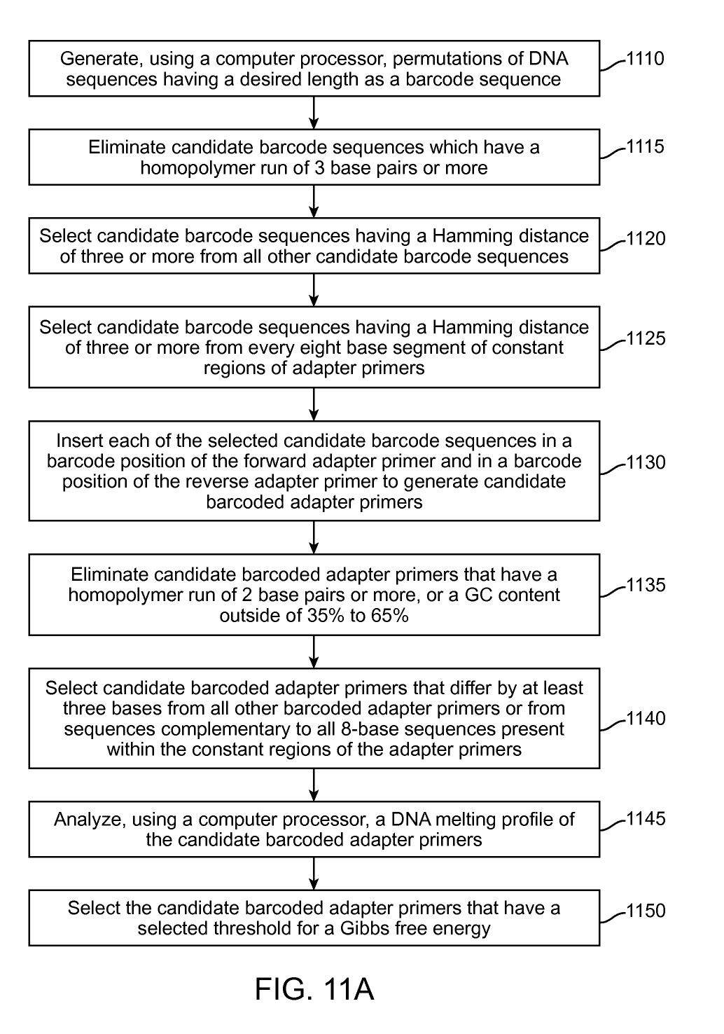

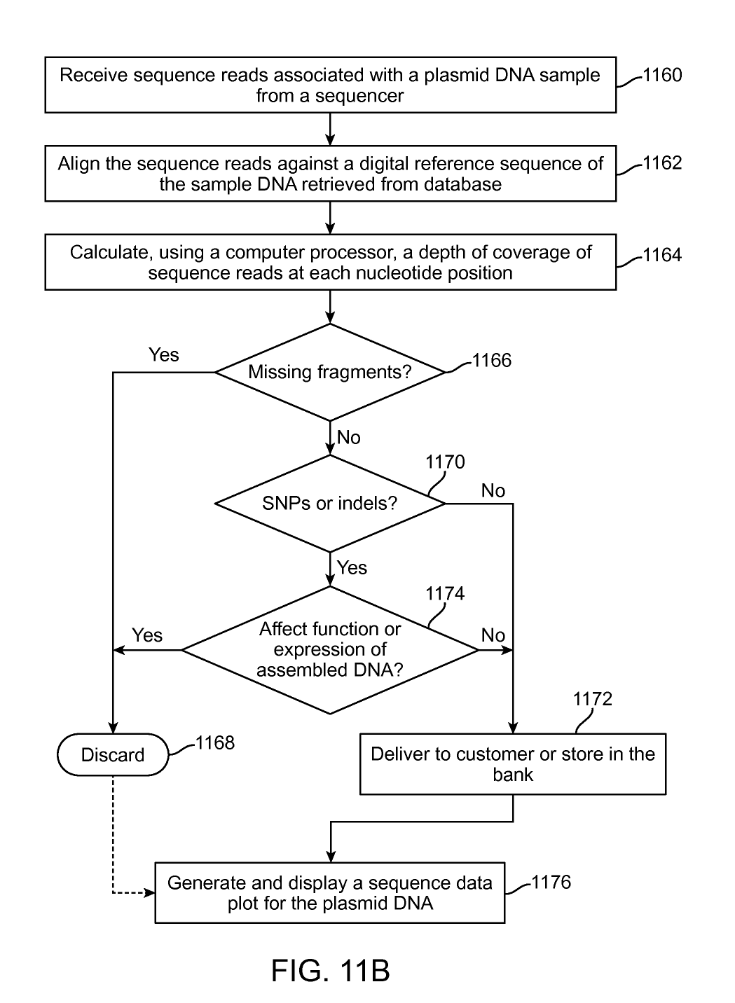

FIG. 11A is a high level schematic diagram illustrating the generation of a set of novel barcode sequences and barcoded adapter primers according to an embodiment of the present invention. In an embodiment, the method of generating a set of suitable barcode sequences and barcoded adapter primers may be performed using one or more processors operated by one or more computer apparatuses such as those illustrated in FIG. 12.

In FIG. 11A, the method includes selecting a desired length for a barcode sequence, and generating, using a computer processor, all permutations of four standard DNA nucleosides (G, A, T, and C) for the desired length (1110). For example, if a barcode sequence of 8 bases in length (L) is desired, then the permutations of 4^{L} (in other words 4⁸) oligonucleotide sequences are generated by considering all permutations of the four standard DNA nucleobases. In an embodiment, Barcrawl algorithm may be used to generate potential barcode sequences. See Frank, BMC Bioinformatics, 2009, 10:362. After generating the 4⁸ permuted oligonucleotide sequences of length 8, the generated sequences are then filtered based on several criteria. For example, it is determined, using the computer processor, whether any candidate index or barcode sequence contains a homopolymer run of 3 base pairs or more (1115). For example, if a candidate barcode has a sequence of ATGCGTTT (SEQ ID NO: 197), then this candidate will be eliminated since it has a homopolymer run of “TTT.”

If the candidate barcode sequence does not include a homopolymer run of 3 base pairs or more, it is determined, using the computer processor, whether every candidate barcode sequence has a Hamming distance of three or more from all other candidate barcode sequences (1120). By definition, the Hamming distance between two strings of equal length is the number of positions at which the corresponding symbols are different. In other words, it is the number of substitutions required to transform one string into another. For example, in the context of a nucleic acid sequence, the Hamming distance between AAGGTTCG (SEQ ID NO: 198) and AAGGCCCG (SEQ ID NO: 199) is 2 since “TT” in the first sequence needs to be replaced with “CC” to transform it into the second sequence. One of these two candidate barcode sequences will be eliminated since they have a Hamming distance of less than three.

The method of generating barcode sequences further includes determining whether every candidate has a Hamming distance of three or more from every eight base segment of the conserved regions of adapter primers. For example, if adapter primers, SEQ ID NOS: 195 and 196, shown below were selected as adapter primer sequences for amplifying tagged polynucleotides, then every candidate must have a Hamming distance of three or more from every eight base segment shown in SEQ ID NOS: 195 and 196.

(Index read 5′ to 3′)
(SEQ ID NO: 195)
5′ AATGATACGGCGACCACCGAGATCTACAC[i5]TCGTCGGCAGCGTC
(Index read 3′ to 5′)
(SEQ ID NO: 196)
5′ CAAGCAGAAGACGGCATACGAGAT[i7]GTCTCGTGGGCTCGG

As an example, if a candidate barcode has a sequence of TTTGATA in step (1125), then this candidate will be eliminated as a potential barcode sequence because it has a Hamming distance of 2 with the first 8 bases (AATGATA) (SEQ ID NO: 200) of the N′ terminal end of SEQ ID NO: 195.

Based on the above steps (1110) through (1125), a novel set of 826 8-base pair candidate indices have been identified. To further optimize the quality of barcode sequences in the context of adapter primers, each of the candidate barcode sequences is inserted into the barcode position of the adapter primers to be used during PCR. For example, if adapter primers shown in SEQ ID NO: 195 and 196 are to be used during PCR (e.g., step (205) of FIG. 2), then each candidate barcode sequence is inserted into position [i5] of SEQ ID NO: 195 (e.g., forward adapter primer) and position [i7] of SEQ ID NO: 197 (e.g., reverse adapter primer) to generate candidate barcoded adapter primers (1130).

In the next few steps, candidate barcoded adapter primers are further analyzed. For example, candidate barcoded adapter primers generated in step (1130) are filtered out if they have mononucleotide runs longer than two bases or a GC content outside of 35% to 65% (1135). The “GC content” refers to the ratio of the number of guanine and cytosine to the total number of all bases in nucleic acids or deoxyribonucleic acids. Then, sequences differing by at least three bases from all other barcoded adapter primers in the set, or from sequences complementary to all 8-base sequences present within the conserved regions of the adapter primers are then selected (1140).

The candidate barcode sequences selected through step (1140) are further filtered by placing them into the context of the full-length adapter primers. For example, each candidate barcode sequence is inserted into position [i5] of SEQ ID NO: 195 and position [i7] of SEQ ID NO: 196. The resulting barcoded adapter primers are analyzed to determine their melting profile. For this step, any suitable DNA melting prediction software, such as DINAMelt, may be used (1145). See Nicholas R. Markham at Rensselaer Polytechnic Institute, which is downloadable from the DINAMelt web site. See, also, Nuc. Acids Res. 2005, vol. 33, W577-W581. The DNA melting prediction software can be used to simulate oligonucleotide melting, and to select those with the lowest predicted tendency to form inter- or intra-molecular duplexes. For example, an oligonucleotide that satisfies a threshold Gibbs free energy may be selected as a final set of barcoded adapter primers (1150). Generally, oligonucleotides that have a more negative Gibbs free energy tend to form inter- or intra-molecular duplexes. Therefore, the stability (Gibbs free energy) may be set at any suitable threshold level (e.g., ΔG=−5) under a typical PCR reaction and salt conditions to filter out unstable barcoded adapter primer candidates.

Using the steps shown in the flowchart of FIG. 11A, 96 “I5-Amy indices” (optimal as i5 indices shown in FIG. 1) and 96 “I7-Amy indices” (optimal as i7 indices in FIG. 1) have been identified. These I5-Amy and I7-Amy indices are shown as SEQ ID NOS: 1-96 and SEQ ID NOS: 97-192, respectively. These 192 unique barcode sequences are optimally designed to be distinguishable during a single sequencing run, and therefore, potentially up to 36,864 DNA samples can be sequenced together. In some embodiments, I5-Amy indices may be used as i5 indices shown in FIG. 1, and I7-Amy indices may be used as i7 indices, allowing 9216 samples to be pooled together for sequencing. So far, more than 4000 libraries have been sequenced together in a single sequencing run. See the Examples section. While these exemplary barcode sequences shown as SEQ ID NOS: 1-192 were selected using the conserved regions of adapter primers of SEQ ID NOS: 195 and 196, any suitable adapter primer sequences may be used to generate other optimal barcode sequences using the method shown in FIG. 11A.

The barcode sequences or barcoded adapter primers generated using the method shown in FIG. 11A can be synthesized using any suitable oligonucleotide synthesis methods. For example, DNA oligonucleotides can be synthesized using solid phase phosphoramidiate chemistry, deprotected and desalted on NAP-5 columns (Amersham Pharmacia Biotech, Piscataway, N.J.) according to routine techniques. See, e.g. Caruthers et al., 1992, Methods Enzymol, 211:3-20. The oligonucleotides can be purified using reversed-phase high performance liquid chromatography. In an embodiment, a request for the barcode sequences or barcoded adapter primers may be transmitted to an oligonucleotide synthesizer shown in FIG. 12. In another embodiment, the oligonucleotides can be custom ordered through a commercial entity, such as IDT (Integrated DNA Technologies, Inc., Coralville, Iowa).

It should be appreciated that the specific steps illustrated in FIG. 11A provide a particular method of generating barcode and adapter primer sequences according to an embodiment of the present invention. Other sequences of steps may also be performed according to alternative embodiments. For example, alternative embodiments of the present invention may perform the steps outlined above as multiple sub-steps as appropriate to the individual step. Furthermore, additional steps may be added or removed depending on the particular applications. Additionally, the features described in other figures or parts of the application may be combined with the features described in FIG. 11A. One of ordinary skill in the art would recognize many variations, modifications, and alternatives.

### Kits and Compositions

In another aspect of the invention, a kit for generating a sequencing library is provided. A kit may comprise a pair of barcoded adapter primers that includes one or more barcoding sequences generated according to embodiments of the present invention. See section 6.3 above. In some embodiments, the barcoded adapter primers may include barcode sequences of SEQ ID NO: 1 through SEQ ID NO: 192. In another embodiment, these barcode sequences can be inserted into adapter primers of SEQ ID NO: 195 and SEQ ID NO: 196 at position [i5] or [i7] to generate barcoded adapter primers. Each of these barcode sequences and barcoded adapter primers is optimally designed to be distinguishable during sequencing using the Illumina or other sequencing platform. Kit embodiments may also include other additional adapter primer sequences which are generated using the method described with reference to FIG. 11A. In certain embodiments, the kit may comprise at least 10, 20, 30, 40, 50, 60, 70, 80, 90, 100, 110, 120, 130, 140, 150, 160, 170, 180, 190, or more different adapter primers.

In some embodiments, the kits may further include reagents that can be used with the present barcoded adapter primers. These kit embodiments may comprise a PCR master mix including one or more standard dNTPs, a DNA polymerase (e.g., Vent polymerase), terminal primers, buffers, and the like. Some kit embodiments may further include reagents for DNA sample preparation, a tagmentation reaction mix, and a transposase removal agent. The kit can further include instructions for the sample preparation, tagmentation reaction and removal of transposases, PCR reactions, sequencing, and the like.

Some kits may further comprise software for processing sequence data. For example, the software may include sorting sequence reads and assigning them to their source (e.g., sample) using the barcode sequences, and aligning and assembling the sorted sequence reads for each sample to generate a consensus sequence of the template polynucleotide in the sample. The software may further include modules to align the sequence reads and/or the consensus sequence to a reference sequence to identify sequence differences (e.g., deletions, indels, mutations, sequencing errors, etc.). The software may further include modules to correct sequencing errors based on the alignment.

### Sequencing

In another aspect, the barcoded polynucleotide fragments prepared and generated in accordance with the present invention can be sequenced using any suitable methods. In an embodiment, a next-generation sequencer can be used to sequence millions of nucleic acid molecules simultaneously. Some platforms rely on sequencing-by-synthesis approach, while other platforms may use sequencing-by-ligation or other approach.

An example of a sequencing technology that can be used in the present methods is the Illumina platform. The Illumina platform is based on amplification of DNA on a solid surface (e.g., flow cell) using fold-back PCR and anchored primers (e.g., capture oligonucleotides). For sequencing with the Illumina platform, DNA is fragmented, and adapters are added to both terminal ends of the fragments. DNA fragments are attached to the surface of flow cell channels by capturing oligonucleotides which are capable of hybridizing to the adapter ends of the fragments. The DNA fragments are then extended and bridge amplified. After multiple cycles of solid-phase amplification followed by denaturation, an array of millions of spatially immobilized nucleic acid clusters or colonies of single-stranded nucleic acids are generated. Each cluster may include approximately hundreds to a thousand copies of single-stranded DNA molecules of the same template. The Illumina platform uses a sequencing-by-synthesis method where sequencing nucleotides comprising detectable labels (e.g., fluorophores) are added successively to a free 3′hydroxyl group. After nucleotide incorporation, a laser light of a wavelength specific for the labeled nucleotides can be used to excite the labels. An image is captured and the identity of the nucleotide base is recorded. These steps can be repeated to sequence the rest of the bases. Sequencing according to this technology is described in, for example, U.S. Patent Publication Application Nos. 2011/0009278, 2007/0014362, 2006/0024681, 2006/0292611, and U.S. Pat. Nos. 7,960,120, 7,835,871, 7,232,656, and 7,115,200, each of which is incorporated herein by reference in its entirety.

In some embodiments, paired end reads may be obtained on nucleic acid clusters on the substrate, where each immobilized polynucleotide is sequenced from both ends of the fragment. Paired end runs read from one end to the other end, and then start another round of reading from the opposite end. In other words, the sequences of the paired reads are read towards each other on opposite strands. When they are aligned against the genome or reference sequence, one read should align to the forward strand, and the other should align to the reverse strand, at a higher base pair position so that they are pointed towards one another. Paired end sequencing runs can provide additional positioning information about the DNA template. Methods for obtaining paired end reads are described in WO/2007/010252 and WO/2007/091077, each of which is incorporated herein by reference.

Another example of a DNA sequencing technology that can be used with the methods of the present invention is SOLiD technology by Applied Biosystems from Life Technologies Corporation (Carlsbad, Calif.). In SOLiD sequencing, DNA may be sheared into fragments, and adapters may be attached to the terminal ends of the fragments to generate a library. Clonal bead populations may be prepared in microreactors containing template, PCR reaction components, beads, and primers. After PCR, the templates can be denatured, and bead enrichment can be performed to separate beads with extended primers. Templates on the selected beads undergo a 3′ modification to allow covalent attachment to the slide. The sequence can be determined by sequential hybridization and ligation with several primers. A set of four fluorescently labeled di-base probes compete for ligation to the sequencing primer. Multiple cycles of ligation, detection, and cleavage are performed with the number of cycles determining the eventual read length.

Another example of a DNA sequencing technology that can be used with the methods of the present invention is Ion Torrent sequencing. In this technology, DNA is sheared into fragments, and oligonucleotide adapters are then ligated to the terminal ends of the fragments. The fragments are then attached to a surface, and each base in the fragments is resolvable by measuring the H⁺ ions released during base incorporation. This technology is described in, for example, U.S. Patent Publication Application Nos. 2009/0026082, 2009/0127589, 2010/0035252, 2010/0137143, and 2010/0188073, each of which is incorporated herein by reference in its entirety.

While three different sequencing technologies are described above, other sequencing platforms and processes can be easily implemented for use with the methods, compositions, and kits described herein.

### Sequence Data Analysis

In another aspect, provided herein is a method of analyzing sequence reads generated by a sequencer using a set of computer-readable instructions or codes (i.e., software). After the sequencer has generated sequenced reads and assigned them to the proper sample, each batch of reads can be aligned to its template (e.g., a digital reference sequence stored in a database). While these functions can be performed by a sequence analyzer module of a sequencer (e.g., Miseq), in some embodiments, these and other functions can be programmed as separate software and performed by a separate computer apparatus dedicated to a sequencer, a user computer and/or a server computer as shown in FIG. 12.

FIG. 11B illustrates a method of analyzing sequence data according to an embodiment of the present invention. In an embodiment, the sequence reads are generated from a plasmid DNA sample, which may include a DNA assembly (i.e., an assembled polynucleotide) inserted into a cloning vector. A DNA assembly or assembled polynucleotide refers to a polynucleotide comprised of two or more component polynucleotide or DNA component of interest. Each component polynucleotide may include a coding sequence, such as a protein-coding sequence, reporter gene, fluorescent marker coding sequence, promoter, enhancer, terminator, or any other naturally occurring or synthetic DNA molecule. A plasmid DNA may further include a vector portion which contains an origin of replication, a multiple cloning site, and a means for selection of host cells harboring the plasmid. Additional description of DNA assemblies can be found in U.S. Pat. Nos. 8,546,136, 8,221,982, 8,110,360, each of which is incorporated by reference in its entirety. In an embodiment, the method shown in FIG. 11B can be used to determine if a plasmid DNA sample comprises a DNA assembly as designed or intended by comparing sequence reads generated from the sequencer with a digital reference sequence of the DNA assembly stored in data storage of a computer system.

In an embodiment, a computer apparatus or system with a user interface may be provided to upload a sample sheet (e.g., csv file) that includes sample and barcode information for each sequencing run on a sequencer. The sequencer assigns each run to the correct sample based on the barcode sequences, and collects the sequence reads in files in a suitable file format (e.g., FASTQ). In the method shown in FIG. 11B, the sequence reads associated with a sample may be received by one of the computer apparatuses or system (e.g., a user computer shown in FIG. 12) (1160). The sequence reads contained in the FASTQ files may be aligned against the associated digital reference sequences (1162). In an embodiment, BWA, a commonly used software package for aligning reads against reference genomes (bio-bwa.sourceforge.net/) may be used. Read alignments may then be stored in a BAM format file, which is the starting point for several downstream analyses. A suitable file format specification is described at the uniform resource locator (URL) samtools.sourceforge.net/SAMv1.pdf.

Referring to FIG. 11B, the method may include generating a folder for each sample by the software, containing sequence information including a pileup file showing the depth of sequence reads at each position of the sequence as well as a variant call file showing single-nucleotide polymorphism (SNPs) or indels along the length of the plasmid. The method may further include calculating the depth of sequence reads at each position of the sequence (1164). In addition, the method includes determining, using the computer processor, whether there are missing fragments in the DNA assembly (1166). The missing fragments may be determined by analyzing the depth of coverage of sequence reads at each position. For example, if there is a missing fragment of 100 base pairs in the DNA assembly, then the depth of coverage at the missing fragment position will be zero. If there are missing fragments (e.g., 10, 20, 30, 40, 50, or more nucleotides), then the plasmid sample may be discarded (1168).

If all DNA components of the DNA assembly are present, then the method further includes analyzing assembled read sequences and the digital reference sequences for smaller differences, for example, single nucleotide polymorphism (SNPs) or indels (e.g., deletions or insertions) (1170). If all of the DNA components are present, then it can be either delivered to a customer who requested the DNA assembly and/or stored in the bank (e.g., freezer) (1172). If there are only small differences between the sequence reads and the digital reference sequence, then the algorithm determines if those differences are in a portion of the plasmid that may affect the function or expression of the genes in the construct (1174). For example, if a change is observed in a linker (e.g., a region of untranslated DNA between two parts), the plasmid containing the DNA assembly may be considered “safe” and may be delivered to the customer or stored in the bank. However, if the variant (e.g., SNPs or indels) is likely to disrupt the intended function (e.g., a premature stop codon in the coding part), it may be flagged as fatal, and the plasmid may be discarded and/or not delivered to the customer.

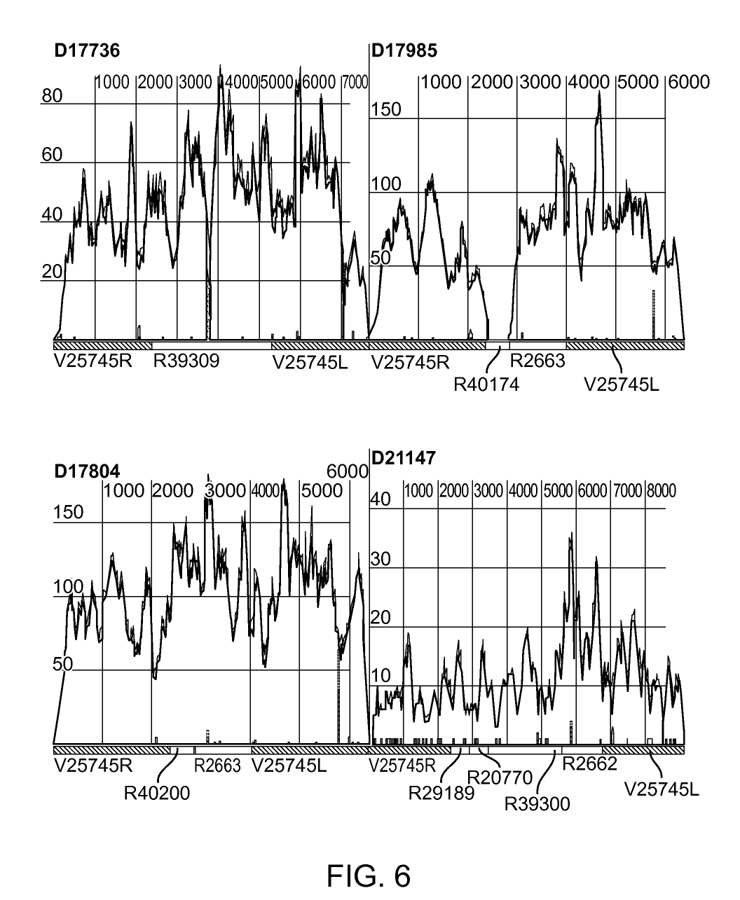

In some embodiments, a sequence data plot for a plasmid DNA can be generated and displayed on a user interface of a computer for each sample (1176). In a sequence data plot, the x-axis may represent the nucleotide position of the plasmid DNA, and the y-axis may represent the depth of coverage for each nucleotide position. Exemplary sequence data plots are illustrated in FIG. 6. As shown in FIG. 6, the spikes or the plotted region show the depth of coverage (e.g., shown in green). A SNP can be represented by colored bars on the plot (e.g., a red bar representing the forward read sequence and a blue bar representing the reverse read). Indels may be represented by different colored bars (e.g., a purple bar indicating an indel in the forward read, and a yellow bar indicating an indel in the reverse read). Also, along the x-axis at a bottom portion of the sequence data plot, DNA assembly parts can be presented in one color (e.g., green), and the vector portion can be presented in another color (e.g., yellow) so that the user can readily recognize if the SNPs or indels are in the vector portion or in the DNA assembly. The color coded sequence data plot allows the user to easily visualize several features associated with the plasmid DNA, such as depth of coverage, positions of missing DNA parts, SNPs, and indels.

In some embodiments, for plasmids containing DNA assemblies stitched from several DNA components, it may be desirable to sequence replicates (e.g., multiple clones) of the plasmid as part of quality control. In these embodiments, the sequence reads from each replicate can be compared against its reference sequence stored in a database. The aligned sequences for each of the replicates can then be compared, and the best replicate (e.g., with read sequences with no deletions, mutations, or substitutions, or the like compared to the reference sequence) may be determined. The method shown in FIG. 11B can also rank the replicates of each assembly based on the number of mutations and their severity, and determine which replicate best matches the digital reference sequence. All data generated by the sequence reads can then be stored in any suitable data storage, such as those exemplified in the computer system of FIG. 12.

In an embodiment, the method shown in FIG. 11B can be used as part of quality control for DNA assembly and sequencing process. For example, when the same SNPs or indels are present in all replicates of a sample (e.g., 4 replicates), or in the same part in different constructs, then they are most likely due to errors in either the digital reference sequence or the template used for PCR amplification of the DNA part. Based on information gathered from the method shown in FIG. 11B, any errors in the digital reference sequence can be corrected, and a source of error in the DNA assembly construct and/or PCR amplification process can be determined and addressed.

It should be appreciated that the specific steps illustrated in FIG. 11B provide a particular method of analyzing sequence data according to an embodiment of the present invention. Other sequences of steps may also be performed according to alternative embodiments. For example, alternative embodiments of the present invention may perform the steps outlined above as multiple sub-steps as appropriate to the individual step. Furthermore, additional steps may be added or removed depending on the particular applications. Additionally, the features described in other figures or parts of the application may be combined with the features described in FIG. 11B. One of ordinary skill in the art would recognize many variations, modifications, and alternatives.

### Computer System

Various methods of the present invention can be performed using one or more computer apparatuses in a computer system. An exemplary computer system 1200 is shown in FIG. 12. One or more computer apparatuses shown in FIG. 12 may be used alone or in combination to perform various methods of the present invention, for example, to generate barcode and adapter primer sequences, and to assemble and analyze sequence data. The computer system 1200 includes a sequencer 1220, which has sequence data receiver module 1221 to obtain sequence read data. The system 1200 also includes an oligonucleotide synthesizer 1230 which includes oligonucleotide data receiver 1231 to receive a request for synthesis of barcode and adapter primer sequences. A server computer 1240 can be used to store or retrieve data, to download software or to execute software remotely. A user computer 1250 can be used by the user to communicate with other computer apparatuses in the computer system 1200 and to transmit, receive, and/or analyze, for example, sequence data or to generate suitable barcode sequences. One or more different entities may operate these computer apparatuses.

All the computer apparatuses shown in FIG. 12 (e.g., the sequencer 1220, the oligonucleotide synthesizer 1230, the server computer 1240, and the user computer 1250) may be operatively linked and can communicate with one another via communication medium 1260. The communication medium 1260 may include wired and/or wireless links. The communication medium 1260 may include the Internet, portions of the Internet, or direct communication links. In some embodiments, the computer apparatuses shown in FIG. 12 may receive data from one another by sharing a hard drive or other memory devices containing the data.

While some of the components of the computer apparatuses are shown in FIG. 12, each computer apparatus may include a number of other components which are not shown in FIG. 12. For example, a PCR chamber in the sequencer 1200 and a reaction chamber in the oligonucleotide synthesizer 1230 are not shown in FIG. 12. In its most basic configuration, a computer apparatus typically includes at least one processor, system memory which may include volatile memory (e.g., random access memory), non-volatile memory (e.g., ROM, flash memory, etc.), or a combination thereof. The memory in any of the computer apparatuses may include computer-readable medium which stores one or more codes or instructions (software) to execute one or more methods or functionalities according to embodiments of the present invention. The codes or instructions for executing the present methods may be stored and/or executed in the same computer apparatus or in more than one computer apparatuses. The codes or instructions may also be transmitted to other computer apparatuses or shared among the computer apparatuses via the communication medium. Each computer apparatus may also include an input device (e.g., keyboard or mouse) and an output device (e.g., a display screen).

The sequencer 1220, in addition to sequence data receiver module 1221 may include sequence analysis module 1222 in memory 1224, a processor 1223, and input/output module 1225. The sequencer data receiver module 1221 may receive a sample sheet (e.g., in csv file) that contains information related to a sample, barcode sequences, and other relevant information for sequence analysis through input/output module 1225 and communication medium 1260. The sequence analysis module 1222 may analyze sequence reads and sort the sequence reads using the barcode sequences and other sample information received in the sequencer data receiver module 1221. The analyzed sequence information may be transmitted to the server computer 1260 and/or the user computer 1250 through the communication medium 1260 for further analysis. Although FIG. 12 illustrates the sequencer 1220 having the sample analysis module 1222, the sequence data may be transmitted to other computer apparatuses, such as the server computer 1240 and/or the user computer 1250 for data analysis.

The oligonucleotide synthesizer 1230, in addition to the oligonucleotide data receiver 1231, may include a synthesis module 1232 in memory 1234, a processor 1233, and input/output module 1235. The oligonucleotide synthesizer 1230 may receive a request to synthesize a barcode sequence, a primer, an adapter, or other nucleotide sequences through the input/output module 1235 and communication medium 1260. The synthesis module 1232 may include software to execute the synthesis of requested oligonucleotides.

The server computer 1240 may include a processor 1241, memory 1242, data storage 1243, and input/output module 1244. The server computer 1240 may interact with other computer apparatuses of the system 1200 and may be used to store data, obtain data, process data, or to output processed and analyzed data to the user computer 1250, sequencer 1220 and/or oligonucleotide synthesizer 1230. For example, reference sequences stored in the data storage 1243 may be retrieved by the user computer 1250 or the sequencer 1220 to compare the digitally stored reference sequences against sequence reads generated by the sequencer 1220.

The user computer 1250 may also include a processor 1251, memory 1252, data storage 1253, and input output device 1256 which may include input/output module 1254 and user interface 1255. The user of the user computer 1250 can communicate with any computer apparatuses of the computer system 1200 via the communication medium 1260. The user of the user computer 1250 may request data or receive data through input/output module 1255 and communication medium 1260. The data, such as sequence alignment and/or sequence coverage data may be analyzed by the server computer 1240 or the user computer 1250, and the analyzed data may be displayed on the user interface 1255 on the user computer. For example, the user computer 1250 may compare sequence reads against a reference sequence for a sample and display sequence data plots as shown in FIG. 6. The user interface 1255 may also illustrate differences between the sequence reads and the reference sequence as well as the depth of coverage for each nucleotide.

Any of the software components or functions described in this application may be implemented as software code to be executed by a processor using any suitable language, such as, for example, Java, C++, or F#. The software code may be stored in a series of instructions, or commands on a computer readable medium, such as random access memory (RAM), a read only memory (ROM), a magnetic medium, such as a hard-drive, or an optical medium such as a CD-ROM. Any such computer readable medium may reside on or within a single computer apparatuses, or may be present on or within different computer apparatuses within a system or network.

### EXAMPLES

### Materials and Methods

### Instrumentation

Liquid transfers were carried out on Biomek FX or NX robots (Beckman Coulter, Brea, Calif.) for volumes greater than 2 μL or on an Echo 550 plus Access robotics (Labcyte, Sunnyvale, Calif.) for volumes less than 2 μL. Sequencing was done on a MiSeq (Illumina, Inc., San Diego, Calif.). Fluorescence was read on an M5 plate reader (Molecular Devices, LLC, Sunnyvale, Calif.). DNA fragment size profiles were determined using either a Bioanalyzer 2100 (Agilent Technologies, Inc., Santa Clara, Calif.) or a Fragment Analyzer (Advanced Analytical Technologies, Inc., Ames, Iowa).

### DNA Assembly and Quantitation

DNA parts with specific linker sequences at each end were assembled in a shuttle vector using yeast homologous recombination, followed by shuttling into Escherichia coli for isolation of DNA, as previously described (Dharmadi et al. (2014) Nucleic Acids Res 42: e22). DNA assemblies built using the ligase cycling reaction (LCR) (de Kok et al. (2014) ACS Synth. Biol. 3: 97-106) were also used in some experiments. Plasmid DNA was prepared by alkaline lysis and silica gel binding (Dharmadi et al., supra) or was amplified using an Illustra Templiphi kit (GE Healthcare Life Sciences, Piscataway, N.J.). DNA concentration was measured using Quant-iT PicoGreen reagent (Life Technologies, Foster City, Calif.) in Costar 3658 or 3677 black 384-well plates (Corning, Inc., Corning, N.Y.). The PicoGreen reagent was diluted with TE (10 mM Tris-HCl, pH 8, 0.5 mM EDTA) containing 0.05% Tween 20.

### Preparing Libraries for Sequencing

As described above, FIG. 2 depicts the chronological workflow for the highly multiplexed plasmid sequencing protocol described here. Using the reagents in an Illumina Nextera kit (FC-121-1031), the tagmentation reaction volume was reduced from 50 μL, as specified in the kit protocol, to 5 μL for the Biomek robots (2 μL of DNA solution and 3 μL of tagmentation master mix containing 0.5 μL tagmentation enzyme and 25 μL tagmentation buffer) or 0.5 μL (200 nL DNA and 300 nL of tagmentation master mix) for the Echo. Rolling circle amplified (RCA) DNA or plasmid DNA prepared by alkaline lysis was diluted with TE to achieve the desired concentration (2.5-10 ng/μL; see Results and Discussion). The transposase was dissociated from the tagmented DNA by adding SDS (sodium dodecylsulfate) to a final concentration of 0.1% (e.g., 125 nL of 0.5% SDS added to 0.5 μL tagmented DNA).

Adapters for the Illumina sequencing process, including 8-base barcodes, were attached to each tagmented DNA sample using 12 cycles of PCR. All primers were obtained from IDT (Integrated DNA Technologies, Inc., Coralville, Iowa) with standard desalting. The barcodes inserted into the Illumina i5 and i7 adapter primer sequences are listed in Table 2. Using the Echo, each sample well received 125 nL of a forward barcode primer and 125 nL of a reverse barcode primer (each at 100 μM). A PCR master mix (24.5 μL) was then added using a Biomek robot. The master mix contained 0.2 units/μL of Vent DNA polymerase (New England Biolabs, Ipswich, Mass.), 1× Thermopol buffer (NEB), 2 mM MgSO₄, 200 μM of each deoxynucleotide triphosphate, and 200 nM of each terminal primer (to mitigate the fact that long oligonucleotides have 5′-end truncations). The thermocycler program was 3 minutes at 72° C., then 12 cycles of 10 seconds at 98° C., 30 seconds at 63° C. and 60 seconds at 72° C. Small fragments and unincorporated primers were removed from the resulting PCR products using 0.6 volume of Ampure XP paramagnetic bead suspension (A63880, Beckman Coulter, Indianapolis, Ind.) per volume of PCR reaction according to the manufacturer's instructions.

Libraries were pooled and normalized based on DNA concentration, and the size of the DNA assembly from which the library was generated. The goal of normalization is to achieve equal molar amounts of the DNA representing each plasmid (see Results and Discussion). The pool was filtered and concentrated using a Microcon Fast-Flow filter unit (EMD Millipore, Billerica, Mass.). The DNA concentration and average fragment size of the pool were determined by Picogreen fluorescence and a high sensitivity DNA chip on a Bioanalyzer 2100, respectively. After diluting the filtered pool to 1.11 nM with water, 18 μL was denatured by adding 2 μL 1N NaOH. After 5 minutes at room temperature, 980 μL ice-cold Illumina Hybridization Buffer was added, followed by 2 μL 1N HCl. The denatured pool was loaded on the MiSeq at 12 pM, which was empirically determined to give the optimum cluster density when following this protocol.

### Sequence Data Processing

A web-based sequencing tracking system was created to manage the many samples and the large amounts of data generated. It facilitates the creation of runs, generation of sample sheets required by the MiSeq, and analysis of multiple data types, including the NGS QC data described here. Reads were demultiplexed using the embedded MiSeq Reporter software. For large numbers of multiplexed samples (greater than 1000), the “File Copy Timeout” setting was increased to avoid premature interruption of the demultiplexing process, which can take several extra hours after a highly multiplexed run appears to have completed. When a sequencing run completes, the system automatically retrieves the FASTQ files from the MiSeqOutput folder. Read mapping to the intended assembly sequences uses BWA v0.6.232 and the “sample” method with default settings. See Li and Durbin (2009) Bioinformatics 25: 1754-1760. Alignments are stored in BAM file format using SAMTOOLS v0.1.19. See Ramirez-Gonzalez et al. (2012) Source Code Biol. Med. 7: 6; Li et al. (2009) Bioinformatics 25: 2078-2079. Mapping statistics are obtained using the SAMTOOLS flagstat utility. A pileup file is generated using SAMTOOLS mpileup with default options to obtain read coverage along the reference sequence.

### Results and Discussions

### Example 1: Reducing Tagmentation Reaction Volume

Table 1 provides an exemplary schematic workflow of next-generation sample preparation. The sample preparation typically has three main phases. In the first phase, tagmentation samples are all normalized to a uniform concentration (1a) and then treated with a fragmentation and labeling enzyme, such as Tn5 transposase pre-loaded with DNA that will flank all template fragments (1b). Once the reaction is complete, the DNA (e.g., tagged polynucleotide fragments) is separated from the tightly-bound transposase in such a way that the template is still competent for PCR (1c). In the second phase, samples are amplified using limited-cycle PCR with primers that contain unique barcodes (2a, b). Once PCR is complete, small high-molarity DNAs that would compete for binding sites on the sequencing surface are removed (2c). In the third phase, the sample concentration and fragment size distribution can be measured and used to normalize the molarity of sequenceable molecules across all samples in certain embodiments (3a).

Tagmentation is like transposon insertion (Reznikoff (2008) Annu Rev. Genet. 42: 269-286), except the transposome cuts the target DNA and appends tags (transposon terminal sequences) to the resulting fragments as shown in FIG. 1. It is a stoichiometric, Poisson process, and the size distribution of the fragments is determined by the ratio of transposome to DNA. An Illumina Nextera kit for preparation of 96 samples costs $7000; therefore, plasmid sequencing with these kits is very expensive and impractical. To reduce cost and establish a manageable workflow, the volume of the tagmentation reaction was reduced in a stepwise fashion, and other steps were modified as necessary to adjust for the reduced sample volume or total DNA mass. The tagmentation step involves combining the DNA template with the transposase, such as Tn5 enzyme, at a suitable protein:DNA ratio. The Tn5 enzyme can be one of the main costs in the sample preparation process. The cost of enzyme ranges from 14 to 19 dollars per microliter at the present value, with 5 microliter of enzymes being recommended per 50 microliters of reaction.

The total amount used per sample can be decreased by scaling down the tagmentation reaction from 50 μL to 0.5 μL. The reduction in volume was performed in a stepwise fashion by modifying other protocol steps as necessary to adjust for reduced samples volume and reduced total mass of DNA.

Since conventional liquid handlers have unacceptable accuracy for handling liquids having a volume of less than 2 μL, a reaction volume of 5 μL (2 μL of DNA and 3 μL of a 1:5 mix of enzyme with 2× reaction buffer) was performed initially. As a first step in reaching this volume, it was determined that dilution of the Tn5 enzyme into 2× reaction buffer prior to addition to the DNA did not significantly affect the sequencing quality. The tagmentation reactions were also performed at a volume of 50 μL, 20 μL, and 10 μL, and no significant difference in sequence quality was observed due to reduction in the tagmentation reaction volume.

As an alternative strategy to overcome the pipetting inaccuracy of conventional liquid handlers (for volumes less than 2 μL), an acoustic liquid handling instrument designed to handle transfers in the nanoliter range was used for the next experiment. Using an acoustic transfer instrument, the tagmentation reaction was performed at 0.5 μL scale.

Early experiments showed that the tagmentation reagents could be used as a master mix and that 5 μL reactions gave sequence data quality equivalent to that obtained using the Nextera kit according to Illumina's protocol (50 μL tagmentation). This remained true upon further reduction of the reaction volume to 0.5 μL using the Echo acoustic liquid dispensing system (Labcyte, Sunnyvale, Calif.).

### Example 2: Removal of Transposases from DNA

After tagmentation, the transposase remains tightly-bound to the DNA (Reznikoff et al. (2008) Annu. Rev. Genet. 42: 269-286) and can inhibit the initial strand-displacing extension required for the PCR. In the Illumina protocol, the tagmented DNA is purified away from the transposase using Zymo Clean and Concentrate columns, but this is impractical for a high throughput process. Thus, other dissociation conditions for removing transposases from nucleic acids were explored. Tagmented DNA fragments or a control reagent (PCR products with ends identical to tagmented fragments after end repair) were subjected to various treatments, and the efficiency of PCR amplification was compared to that using Zymo column purification.

Five treatment possibilities were explored: 1) dilution with TE buffer; 2) dilution with TE buffer and heat; 3) SDS and Triton; 4) high pH and neutralization; and 5) chaotropic salts+dilution. These treatments were compared to Zymo treated samples using a simple experimental system, which compared the post-PCR yield of either plasmid DNA that had been fragmented by Tn5 protein or linear DNA that was not exposed to Tn5 protein but was still flanked by the same terminal primer binding sites.

In the first two treatments, the following conditions were compared with the Zymo kit: 1) dilution with TE buffer; and 2) dilution with TE buffer and heat. Pooled tagmentation reactions were split between the three treatments. The Zymo samples were prepared according the Zymo kit protocol. Samples for the dilution treatments were diluted by adding 90 μL of TE to 10 μL of tagmentation reaction. Samples for the first treatment stayed at room temperature (25-27° C.) for 10 minutes while samples for the second treatment were incubated at 68° C. for 10 minutes. All samples were used in 10-cycle PCR reactions with a common pair of barcode primers and, after cleaning up PCR reaction products with Ampure beads to remove small DNA fragments, the cleaned up PCR reaction products were compared on an Agilent Bioanalyzer.

The results indicated that none of these treatments inhibited the PCR reaction, and the Zymo kit treatment produced the highest PCR yield. Amplification of the linear DNA, which tested for inhibition of the PCR reaction, was statistically indistinguishable for the three conditions (lowest P=0.07): 1) dilution of the tagmentation reaction mixture with TE yielded 0.80 times as much DNA as the Zymo kit; and 2) dilution of the tagmentation reaction mixture with TE and heat yielded 0.92 times as much as the Zymo kit (Data not shown). Amplification of the tagmented plasmid DNA (which tested removal of the Tn5 protein) revealed a doubling in DNA yield for each treatment from the worst treatment to the best treatment: 1) the dilution of the tagmentation reaction mixture with TE resulted in a DNA yield which is 0.28 times as much as that of the Zymo kit; and 2) the dilution of the tagmentation reaction mixture with TE and heat resulted in a DNA yield which is 0.53 times as much as that of the Zymo kit (Zymo kit=1±0.04X). While a simple treatment such as diluting the tagmentation reaction mixture with TE and heat provided 50% as much DNA as the Zymo kit, the better treatment conditions that can yield higher DNA yields were explored in the next set of experiments.

The third treatment explored was the addition of SDS to remove protein followed by addition of Triton X-100 (triton) to sequester the SDS. As before, pooled tagmentation reactions were split between different Tn5-removal treatments. A matrix of 24 SDS/triton treatments was prepared, where each sample received one of 6 different SDS solutions and one of 4 different triton solutions. The Zymo kit samples were processed according to the manufacturer's protocol. Non-Zymo reactions were incubated at 75° C. for 10 minutes after addition of SDS, amended with triton in TE, and mechanically shaken. All reactions were then used in identical PCR reactions and compared by Fragment Analyzer.

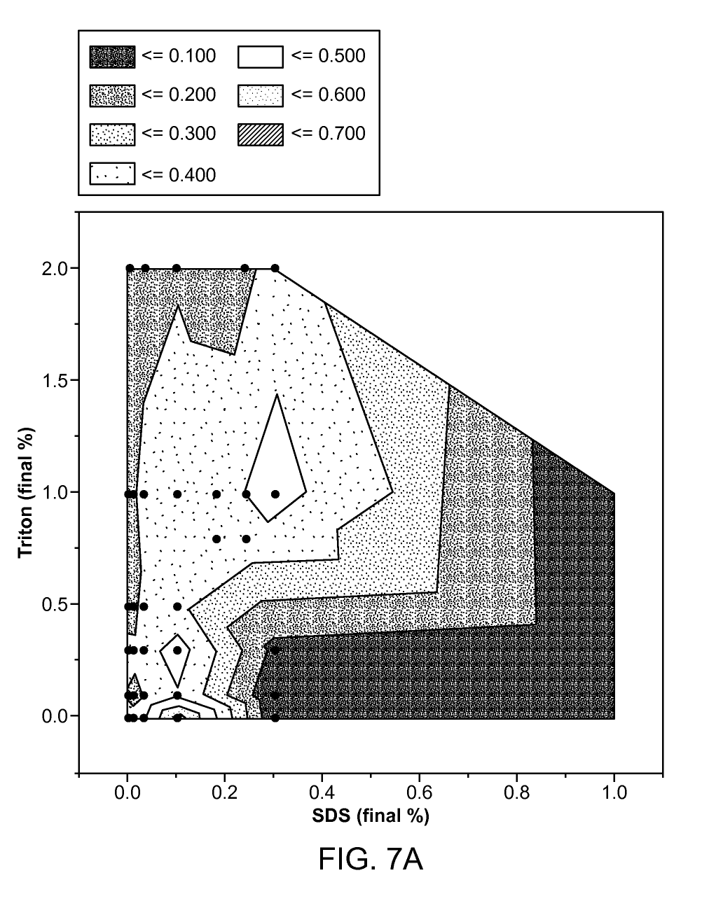

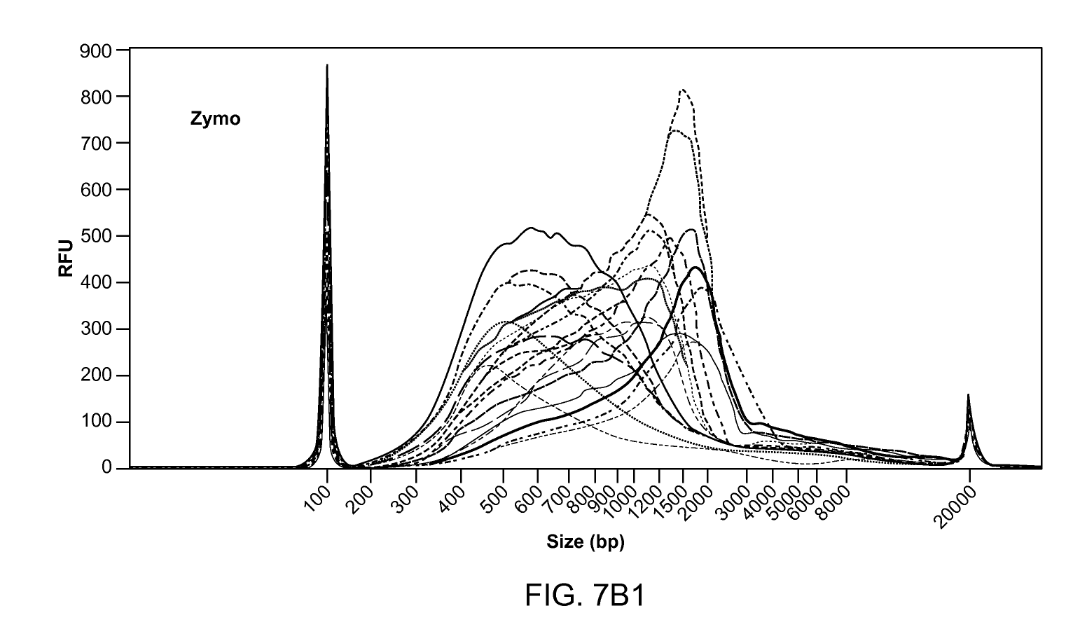

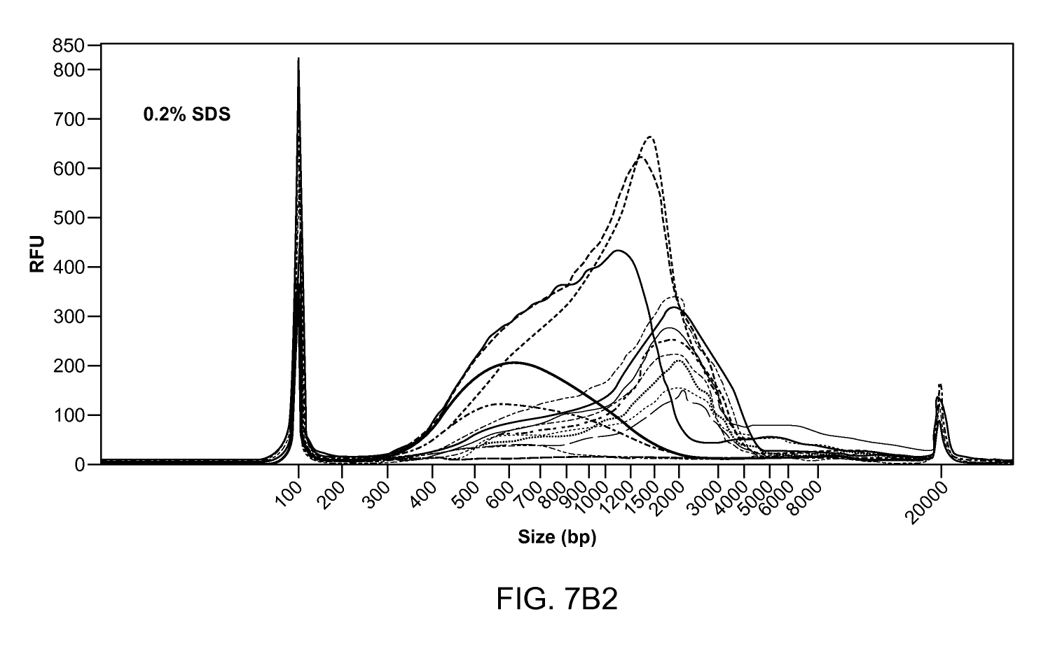

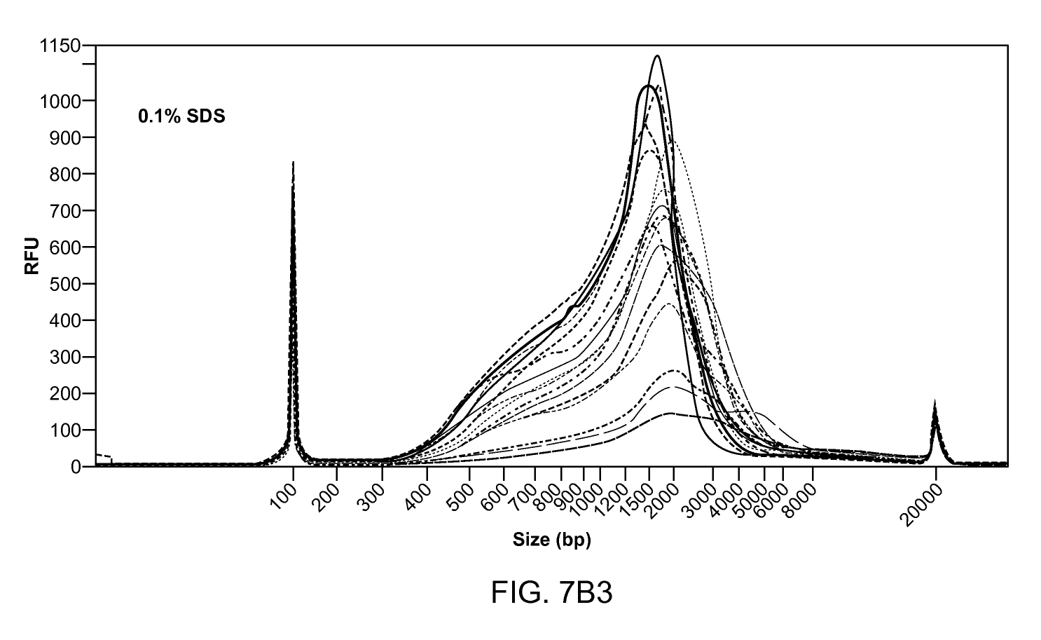

The experimental results of the third treatment are illustrated in FIG. 7A. In FIG. 7A, the operational range and the optimum SDS and Triton X-100 concentrations were identified for removal of the transposase after tagmentation. FIG. 7A shows a response surface plot of the concentration of DNA amplified by PCR relative to that obtained using Zymo column purification. The DNA concentration in a selected size was determined using a Bioanalyzer. SDS was added to the tagmentation reaction to different final concentrations, as shown along the horizontal axis, followed after 10 minutes at 75° C. by dilution with Triton X-100 (“triton”) solutions giving concentrations between 0 and 2%, as shown along the vertical axis. The black dots are the actual data points specified by the design of the experiment using JMP (SAS Institute, Inc. Cary, N.C.).

For the linear DNA (data not shown), the recovery of DNA increased slightly with lower concentrations of SDS: at 0% SDS, the DNA yield was 0.96 times as much as the Zymo treated sample; at 0.1% SDS, the DNA yield was 1.1 times as much as the Zymo treated sample; at 0.2% SDS, however, the DNA yield dropped to 0.1 times as much as the Zymo treatment sample, indicating PCR inhibition. The addition of triton after the SDS treatment ameliorated the inhibition of the PCR reaction even when the SDS concentrations were as high as 0.3%.

For the tagmented plasmid (FIG. 7A), the maximum recovery of DNA observed was at 0.1% SDS, 0% triton. The ability of triton to ameliorate PCR inhibition by SDS was also apparently present for these samples. However, since the total DNA recovery never exceeded that seen with 0% triton, the more operations-friendly treatment condition of 0.1% SDS, 0% triton was adopted in removing transposases in some embodiments. Also, it was later found that heating to a temperature of 75° C. was unnecessary for this treatment condition.

The fourth and fifth treatment conditions, high pH and guanidine isothiocyanate, also resulted in a reasonable amount of DNA recovery. These treatment conditions, however, did not improve recovery of DNA as compared to the SDS treatment. The fourth and fifth treatment conditions were not further explored as they may add operational challenges in some circumstances. As a note, it was discovered that samples incubated with guanidine isothiocyanate at room temperature had statistically indistinguishable recovery of DNA compared to samples incubated at a temperature of 68° C. This result indicated that heating samples, an operationally challenging step, was not necessary. As noted above, it was also later discovered that heating was unnecessary for the SDS treatment conditions for the maximum recovery of DNA.

After completing the five different treatment conditions, the treatment conditions with SDS were further explored. Experimental conditions were designed to further increase DNA recovery. In the designed experiments, a number of different conditions were varied: the SDS concentration was varied; the incubation temperature was varied; the sample was diluted to 50 μL instead of 100 μL to add twice as much DNA to the PCR reaction. The only sample that showed the reduced PCR efficiency was the one containing the highest amount of SDS (0.02% in the PCR). No adverse effect was found from the SDS concentration or dilution in any other samples. However, a large effect was found from the incubation temperature: Incubation at 75° C. returned, as before, 0.53 time as much as the Zymo treatment; incubation at 50° C. returned 0.87 times as much as the Zymo treatment; and incubation at 25° C. returned an average of 0.98 times as much as the Zymo treatment. Therefore, the following conditions were selected as optimum treatment conditions: 0.1% SDS and 25° C.

To verify that this modified sample preparation protocol resulted in high-quality sequence data, a set of 32 plasmids was treated three ways: 1) by Zymo kit; 2) with 0.1% SDS (final concentration); or 3) with 0.2% SDS (final concentration). Samples from all three treatments were uniquely barcoded but otherwise put through identical PCR reactions, purified, analyzed by Fragment Analyzer, normalized, pooled, and sequenced.

It was first verified that samples prepared with these new SDS-based conditions returned as much DNA after barcoding PCR reactions as samples prepared with the Zymo kit. The tagmented SDS-treated plasmid samples in this experiment (n=15) returned an average of 1501±169 ng while the average DNA returned for Zymo column samples (n=16) was 1412±206 ng.

As a note, it was discovered that the distribution of fragment sizes was significantly different between samples treated with SDS and with the Zymo kit. This is illustrated in FIGS. 7B1 through 7B3. FIGS. 7B1 through 7B3 show superimposed fragment analyzer traces of samples treated with 1) Zymo kit; 2) 0.2% SDS (final concentration); 3) 0.1% SDS (final concentration). All samples were incubated at room temperature. The DNA fragment size is shown along the horizontal axis, and the DNA concentration is shown along the vertical axis (RUF=relative fluorescence units). The DNA treated with the Zymo kit was broadly distributed between roughly 400 base pairs and 2000 base pairs (FIG. 7B1). The DNA samples treated with SDS had less than 25% of their DNA mass below 600 base pairs, and the majority in a large peak centered around 2000 base pairs (FIG. 7B3). Because the sequencing process favors molecules in the 300-800 base pair range, it was found that this altered distribution may necessitate adjusting the PCR extension time to favor smaller fragments as well as revising the normalization and dilution calculations so that the same number of sequenceable DNA fragments reaches the sequencer regardless of the shape of the distribution.

The sequence data revealed two groups of statistically significant differences between Zymo-treated and SDS-treated samples. The first group of results is rooted in the insert size. The Zymo-treated samples contained, on average, a larger fraction of fragments that were smaller than 150 base pairs. Because these small fragments are informatically discarded, the final sequence metrics are strongly affected. The second group of results related to how evenly sequence data is distributed across the plasmids. Surprisingly, it was discovered that coverage was significantly more evenly distributed across SDS-treated samples than across Zymo-treated samples (P<0.0001). Specifically, the coefficient of variation (CV) of sequence depth was 25% for Zymo-treated samples but 20% and 18% for the 0.2% and 0.1% SDS-treated samples, respectively. This unexpected difference is valuable because it will allow increased plexity; the reduced variability will in turn decrease the average coverage required to meet the sequence quality specification. Thus, while other dissociation conditions can be used to remove transposases from DNA, the addition of SDS to a final concentration of 0.1% was found to be most effective at removing the transposase without interfering with the subsequent PCR. This discovery and other suitable treatment conditions led to elimination of the cost-prohibitive column spin step during sample preparation for sequencing in certain embodiments.

### Example 3: Barcoding PCR

Unique barcodes can be added to every DNA fragment at one or both ends. The specific sequence or “index” used as a barcode sequence is unrestricted, though the field has established a precedent of 8-bp indices. Each index can be used for either of the two ends, which have slightly different sequences added by the Tn5 protein and are referred to as the i5 and i7 ends.

To enable the required level of multiplexing, a set of barcode adapter primers was designed using previously described algorithms (Bystrykh (2012) PLoS One 7: e36852; Frank (2009) BMC Bioinformatics 10: 362). The structure of the i5 and i7 index primers was maintained, but in order to reach higher plexity, a novel set of 826 8-base pair candidate indices were identified using the following criteria: (1) no index contained a homopolymer run of 3 base pairs or more; (2) every candidate index has a Hamming distance of three or more from all other indices; and (3) every candidate has a Hamming distance of three or more from every eight base segment of the conserved sections of the i5 and i7 sequence. These candidate indices were then used to generate the corresponding candidate i5 and i7 barcode primers. From all possible 8-base sequences generated, those with mononucleotide runs longer than two bases or GC content outside the range of 35% to 65% were removed. The following sets of sequences were then selected: sequences differing by at least three bases from all other barcodes in the set, or from sequences complementary to all 8-base sequences present within the conserved regions of the i5 and i7 adapter primers. These sequences (approximately 800) were then placed into the context of the full-length Illumina adapter primer, and the resulting adapter primers were analyzed using DINAMelt (Markham (2005) Nucleic Acids Res. 33: W577-581) to predict the stability (Gibbs free energy) of each folded polynucleotide. In other words, the resulting adapter primers were examined to find those with the lowest predicted tendency to form inter- or intra-molecular duplexes.

Table 2 lists the set of barcode sequences generated by the method described above. These barcode sequences were custom ordered from Integrated DNA Technologies, and were used in highly multiplexed sequencing experiments.

TABLE 2
Barcode Sequences

| | | SEQ | | | SEQ |
|---|---|---|---|---|---|
| Name | Barcode | ID NO: | Name | Barcode | ID NO: |
| I5- | CCATGTTG | 1 | I7- | GCTGTGTT | 97 |
| Amy_3 | | | Amy_1006 | | |
| I5- | ACACCGGC | 2 | I7- | ATGGCGAC | 98 |
| Am _6 | | | Amy_1017 | | |
| I5- | GTATCCTA | 3 | I7- | GGCGAGCA | 99 |
| Amy_11 | | | Amy_1018 | | |
| I5- | GGATGAGC | 4 | I7- | GCTGTCCG | 100 |
| Amy_13 | | | Amy_1019 | | |
| I5- | GAGACTAG | 5 | I7- | CGAGTGAA | 101 |
| Amy_14 | | | Amy_1030 | | |
| I5- | GGCCTCTA | 6 | I7- | CCATCACT | 102 |
| Amy_15 | | | Amy_1033 | | |
| IS- | CAATGATA | 7 | I7- | AGTACACC | 103 |
| Amy_17 | | | Amy_1036 | | |
| I5- | CCTATCCA | 8 | I7- | TCGCTGAT | 104 |
| Amy_21 | | | Amy_1049 | | |
| I5- | TTGATATA | 9 | I7- | GGCGGTAA | 105 |
| Amy_22 | | | Amy_1052 | | |
| I5- | AGCGATAT | 10 | I7- | CCGCCGAA | 106 |
| Amy_23 | | | Amy_1056 | | |
| I5- | CCTACAGT | 11 | I7- | GATTGCGA | 107 |
| Amy_26 | | | Amy_1057 | | |
| I5- | ATGACAGT | 12 | I7- | ACATTCTC | 108 |
| Amy_27 | | | Amy_1058 | | |
| I5- | AGTGTACA | 13 | I7- | CCACTGGT | 109 |
| Amy_30 | | | Amy_1065 | | |
| I5- | CTGGCACG | 14 | I7- | CCTGCCAA | 110 |
| Amy_31 | | | Amy_1078 | | |
| I5- | CGCCTAAC | 15 | I7- | ATACGTCC | 111 |
| Amy_37 | | | Amy_1080 | | |
| I5- | CTCGTCGT | 16 | I7- | TCAACTCT | 112 |
| Amy_38 | | | Amy_1091 | | |
| I5- | TACAGACA | 17 | I7- | ACCGCTAC | 113 |
| Amy_40 | | | Amy_1095 | | |
| I5- | CAGTACCA | 18 | I7- | GCAATGCT | 114 |
| Amy_41 | | | Amy_1097 | | |
| I5- | AAGGTATC | 19 | I7- | GGACCGCG | 115 |
| Amy_45 | | | Amy_1100 | | |
| I5- | AATTGAAT | 20 | I7- | CCTACTTA | 116 |
| Amy_46 | | | Amy_1101 | | |
| I5- | CGCAAGAG | 21 | I7- | GATGATCT | 117 |
| Amy_47 | | | Amy_1102 | | |
| I5- | CTCGATAA | 22 | I7- | CAGTGGAA | 118 |
| Amy_48 | | | Amy_1112 | | |
| I5- | TTGTTCTC | 23 | I7- | GTTGACAT | 119 |
| Amy_49 | | | Amy_1115 | | |
| I5- | TGACATCT | 24 | I7- | GCCATAGA | 120 |
| Amy_50 | | | Amy_1125 | | |
| I5- | TTCTGTTC | 25 | I7- | TCTGGAAT | 121 |
| Amy_52 | | | Amy_1134 | | |
| I5- | TCAGCACC | 26 | I7- | TGCCGATC | 122 |
| Amy_54 | | | Amy_1160 | | |
| I5- | GTTATCAC | 27 | I7- | ATGTAGCA | 123 |
| Amy_56 | | | Amy_1173 | | |
| I5- | ACGTGTCC | 28 | I7- | GTCACCAA | 124 |
| Amy_57 | | | Amy_1174 | | |
| I5- | TGGCTCCT | 29 | I7- | CTAAGAGT | 125 |
| Amy_60 | | | Amy_1193 | | |
| I5- | ATGCGAAG | 30 | I7- | CCTCTCTC | 126 |
| Amy_61 | | | Amy_1195 | | |
| I5- | AACTACCT | 31 | I7- | GCTAATGA | 127 |
| Amy_62 | | | Amy_1199 | | |
| I5- | TGGTCATA | 32 | I7- | CTGGTGAT | 128 |
| Amy_65 | | | Amy_1203 | | |
| I5- | ACATAACA | 33 | I7- | CTGATAGC | 129 |
| Amy_68 | | | Amy_1209 | | |
| I5- | ACAGGCAT | 34 | I7- | AATGCCGG | 130 |
| Amy_69 | | | Amy_1214 | | |
| I5- | ACCTCTCT | 35 | I7- | GCACATTG | 131 |
| Amy_72 | | | Amy_1230 | | |
| I5- | AGATGATT | 36 | I7- | TGTTGCAC | 132 |
| Amy_88 | | | Amy_1233 | | |
| I5- | AGACTCTT | 37 | I7- | GCCTATCG | 133 |
| Amy_92 | | | Amy_1234 | | |
| I5- | ACTAGCAG | 38 | I7- | GCCTTCGG | 134 |
| Amy_93 | | | Amy_1244 | | |
| I5- | ATACACGT | 39 | I7- | TCGGTGTC | 135 |
| Amy_95 | | | Amy_1249 | | |
| I5- | ACAGCATT | 40 | I7- | GCGACGTA | 136 |
| Amy_96 | | | Amy_1258 | | |
| I5- | ATAATTAG | 41 | I7- | GCACGATT | 137 |
| Amy_102 | | | Amy_1269 | | |
| I5- | TCTAGACC | 42 | I7- | CTGCTACT | 138 |
| Amy_108 | | | Amy_1272 | | |
| I5- | CTACAGAC | 43 | I7- | GCTTACAA | 139 |
| Amy_109 | | | Amy_1274 | | |
| I5- | CTAGTTGC | 44 | I7- | TGAACAAC | 140 |
| Amy_111 | | | Amy_1276 | | |
| I5- | CATTGTAC | 45 | I7- | ACTTGTAA | 141 |
| Amy_115 | | | Amy_1281 | | |
| I5- | AGTATGAT | 46 | I7- | TCTGCGAC | 142 |
| Amy_116 | | | Amy_1310 | | |
| I5- | AGAATCAA | 47 | I7- | GGCCGAGT | 143 |
| Amy_117 | | | Amy_1311 | | |
| I5- | TTCATTGA | 48 | I7- | CACATTAC | 144 |
| Amy_118 | | | Amy_1312 | | |
| I5- | ATCACTTA | 49 | I7- | GATTCCAG | 145 |
| Amy_120 | | | Amy_1321 | | |
| I5- | TCAATCAT | 50 | I7- | TCCGCGGT | 146 |
| Amy_121 | | | Amy_1331 | | |
| I5- | AGATGTCA | 51 | I7- | ACTGACGA | 147 |
| Amy_125 | | | Amy_1343 | | |
| I5- | GTAATATG | 52 | I7- | GCACACAT | 148 |
| Amy_127 | | | Amy_1351 | | |
| I5- | CCACAGCA | 53 | I7- | CCTAGGAT | 149 |
| Amy_129 | | | Amy_1354 | | |
| I5- | CATCCACC | 54 | I7- | CTTAACGA | 150 |
| Amy_131 | | | Amy_1356 | | |
| I5- | CAGACTCA | 55 | I7- | CCACCATC | 151 |
| Amy_133 | | | Amy_1357 | | |
| I5- | ACTTCATA | 56 | I7- | TGAGCCGC | 152 |
| Amy_135 | | | Amy_1359 | | |
| I5- | CCAACGGA | 57 | I7- | TACTCCAC | 153 |
| Amy_137 | | | Amy_1366 | | |
| I5- | ACCAATCC | 58 | I7- | GGCAGCCG | 154 |
| Amy_141 | | | Amy_1369 | | |
| I5- | TAGCATAA | 59 | I7- | TTCGACTC | 155 |
| Amy_145 | | | Amy_1375 | | |
| I5- | TGACAGGA | 60 | I7- | TACGAATA | 156 |
| Amy_146 | | | Amy_1386 | | |
| I5- | CATGAAGT | 61 | I7- | AGGTCCTT | 157 |
| Amy_147 | | | Amy_1392 | | |
| I5- | TATAGTAG | 62 | I7- | CAGCGAGG | 158 |
| Amy_150 | | | Amy_1397 | | |
| I5- | ACCACATC | 63 | I7- | GACCTCAG | 159 |
| Amy_152 | | | Amy_1398 | | |
| I5- | AATTATAG | 64 | I7- | GCTAGGCG | 160 |
| Amy_153 | | | Amy_1408 | | |
| I5- | TTCCACAT | 65 | I7- | TGCACGGA | 161 |
| Amy_156 | | | Amy_1414 | | |
| I5- | CAGGCATA | 66 | I7- | TAACGACC | 162 |
| Amy_158 | | | Amy_1427 | | |
| I5- | TAGTTAAC | 67 | I7- | AACGGTTC | 163 |
| Amy_162 | | | Amy_1436 | | |
| I5- | CCGCATCT | 68 | I7- | TGCAATGC | 164 |
| Amy_163 | | | Amy_1437 | | |
| I5- | ATGAATCT | 69 | I7- | ATTCGAGC | 165 |
| Amy_164 | | | Amy_1439 | | |
| I5- | TGTGACTT | 70 | I7- | TGCGTTCC | 166 |
| Amy_168 | | | Amy_1440 | | |
| I5- | AGGCTTAC | 71 | I7- | ATGATCCA | 167 |
| Amy_169 | | | Amy_1447 | | |
| I5- | CTGTCCTG | 72 | I7- | GGAACGAT | 168 |
| Amy_170 | | | Amy_1448 | | |
| I5- | GATACATT | 73 | I7- | TCCGAAGC | 169 |
| Amy_171 | | | Amy_1451 | | |
| I5- | ACCGGAGT | 74 | I7- | CTGCCAAC | 170 |
| Amy_172 | | | Amy_1453 | | |
| I5- | TGACCTTC | 75 | I7- | AACCGCGG | 171 |
| Amy_173 | | | Amy_1462 | | |
| I5- | AGGACTAA | 76 | I7- | AGAGCGAG | 172 |
| Amy_177 | | | Amy_1466 | | |
| I5- | TCATTGAC | 77 | I7- | TCGTATGT | 173 |
| Amy_183 | | | Amy_1470 | | |
| I5- | CAGGACAT | 78 | I7- | CTCGCTTC | 174 |
| Amy_184 | | | Amy_1473 | | |
| I5- | TAATACTC | 79 | I7- | TGGAGCGC | 175 |
| Amy_185 | | | Amy_1490 | | |
| I5- | TATGCTTC | 80 | I7- | GTGGCCGT | 176 |
| Amy_187 | | | Amy_1491 | | |
| I5- | TTAGGAGA | 81 | I7- | TGGCCACC | 177 |
| Amy_195 | | | Amy_1493 | | |
| I5- | GGCTAAGA | 82 | I7- | GCGCAGTT | 178 |
| Amy_199 | | | Amy_1506 | | |
| I5- | TAGTGAGT | 83 | I7- | TCTCCGTA | 179 |
| Amy_201 | | | Amy_1507 | | |
| I5- | CCATCACT | 84 | I7- | GCGTTGCG | 180 |
| Amy_207 | | | Amy_1509 | | |
| I5- | TTATAGTT | 85 | I7- | GATAGCAT | 181 |
| Amy_208 | | | Amy_1511 | | |
| I5- | AGTACACC | 86 | I7- | AACCAGGT | 182 |
| Amy_210 | | | Amy_1512 | | |
| I5- | CACTTGAG | 87 | I7- | CATGACTA | 183 |
| Amy_211 | | | Amy_1536 | | |
| I5- | AGTCCAAG | 88 | I7- | GTCTCGGA | 184 |
| Amy_213 | | | Amy_1541 | | |
| I5- | TCACTACA | 89 | I7- | CTCTAAGT | 185 |
| Amy_215 | | | Amy_1543 | | |
| I5- | AGAATTCC | 90 | I7- | CATCGTGT | 186 |
| Amy_216 | | | Amy_1560 | | |
| I5- | AATTAAGC | 91 | I7- | GCAACCTT | 187 |
| Amy_218 | | | Amy_1574 | | |
| I5- | ACACCTAT | 92 | I7- | GAGATTCT | 188 |
| Amy_219 | | | Amy_1577 | | |
| I5- | ATTGCAAT | 93 | I7- | CACTGCTT | 189 |
| Amy_221 | | | Amy_1586 | | |
| I5- | TGGATAAT | 94 | I7- | AGGTACGA | 190 |
| Amy_225 | | | Amy_1621 | | |
| I5- | CAATCGTC | 95 | I7- | ACCGAGTC | 191 |
| Amy_250 | | | Amy_1635 | | |
| I5- | TAGAAGTC | 96 | I7- | CACAAGTA | 192 |
| Amy_256 | | | Amy_1645 | | |

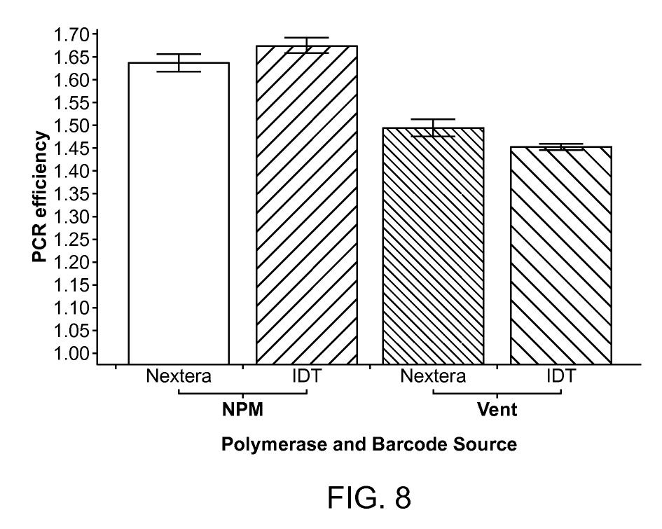

FIG. 8 illustrates that the custom barcode primers ordered from Integrated DNA Technologies and barcode primers ordered from Illumina gave equivalent PCR efficiencies. At least 192 forward and 192 reverse barcode sequences (providing 36,864 unique barcode combinations) pass the filtering process described above. More specifically, PCR efficiency was compared using Vent polymerase and custom primers ordered from IDT, or the Nextera kit reagents NPM (Nextera PCR master mix) and PPC (PCR primer cocktail). The template for the PCR reaction was tagmented DNA which was generated following the Illumina Nextera kit protocol. PCR efficiency is defined as ([DNA]_{final}/[DNA]ᵢₙᵢₜᵢₐₗ)^{(1/N)}, where N is the number of cycles of PCR. Perfect efficiency is 2, and no amplification is 1. The concentration of DNA in a chosen size range before and after PCR was measured with a Bioanalyzer 2100 and a high sensitivity chip.

For the experiments shown in FIG. 8, the barcoded adapters are attached to the ends of Nextera library fragments using a non-standard PCR protocol (shown in FIG. 1) requiring initial end repair with a strand-displacing polymerase. The volume of this PCR cannot be reduced too much. Otherwise, the subsequent size-selection by solid phase reversible immobilization may not be operationalized. By reducing the tagmentation reaction volume, the PCR reagents in the Nextera kit may become limiting. As a potential replacement reagent to carry out this PCR, Vent polymerase was chosen from New England Biolabs, which is reported to have strand displacement activity and a relatively high fidelity (Kong et al. (1993) J. Biol. Chem. 268: 1965-1975). FIG. 8 shows that Vent polymerase can replace the NPM reagent in the Illumina Nextera kit with only a slight decrease in PCR efficiency, which could be remedied by a compensatory increase in the number of PCR cycles.

The performance of Vent-based master mix according to the present invention was compared to the Illumina Nextera PCR Mastermix (NPM). It was found that there were two differences. The first difference was that NPM samples tend to have a larger fraction of DNA smaller than 400 base pairs while Vent samples tend to have a larger fraction between 500 base pairs and 1000 bp (P=0.025). The second difference was that NPM samples had roughly double the DNA concentration of Vent samples. (Data not shown). A two-fold difference after 8 cycles suggests that, in each cycle, NPM is 10% more efficient than Vent (i.e., 1.1⁸=2.1). Further experiments showed that this difference in DNA yield could be ameliorated by adding one or two PCR cycles to reactions using Vent polymerase.

It was also found that the concentration of barcode primer also had a large effect on the DNA yield for Vent-based master mix. Experiments that used Vent-based master mix and 0.1 μM barcode primer yielded less than 5% as much as the equivalent NPM reaction (data not shown). When barcode primers were used at or above 0.5 μM, the DNA yield of Vent-based master mix reached a plateau of 45% as much as the equivalent NPM reaction. The yield of NPM reactions remained unchanged across this concentration range (data not shown). It was found that there was no statistical difference in DNA yield or in the fragment size distribution between NPM reactions using the Illumina barcode primers and NPM reactions using the barcode primers according to the present invention.

It was tested whether the Vent-based PCR master mix would adversely affect sequence quality by preparing and sequencing a set of 42 recently-constructed plasmids using either NPM or Vent and using both presently designed and Illumina-provided barcode primers. Because of the difference in polymerase efficiency, NPM samples were given 8 cycles of PCR, and Vent samples were given 10 cycles of PCR. No statistically significant difference was found in any of the sequence quality metrics, including the number or quality of mutations identified, between samples prepared with NPM and sample prepared with the Vent-based master mix. Similarly, the origin of barcode primer resulted in no statistically significant difference in any sequence quality metric. Based on this data, it was concluded that the Vent-based master mix according to the present invention performs at least as well as a commercially available alternative, Illumina NPM, as long as additional PCR cycles compensate for the lower DNA yield.

### Example 4: Source of DNA for the Library Preparation

For preparing plasmid DNA, rolling circle amplification (RCA) takes less than a third the hands-on time and produces more consistent final DNA concentrations compared to plasmid minipreps (Dean et al. (2001) Genome Res 11: 1095-1099). In particular, rolling circle amplification (RCA) of plasmids using Phi29 polymerase generates large amounts of linear high molecular weight concatamers of the plasmid. This is a much less labor intensive way to obtain DNA than plasmid minipreps, which involve multiple centrifugation steps. Furthermore, RCA gives good Sanger sequence data (Dean et al. (2001) Genome Res 11: 1095-1099), good restriction digest banding (Dharmadi et al. (2014) Nucleic Acids Res. 42: e22), and whole genome-amplified DNA provides good Illumina sequence data (Indap et al. (2013) BMC Genomics 14: 468).

A set of 384 DNA assemblies ranging in size from 4 kb to 20 kb were used to prepare both RCA DNA and plasmid DNA, and the 768 DNA samples were used to prepare a pool of 768 Nextera libraries for the MiSeq. FIG. 3A illustrates distribution and statics of average depth of coverage per sample (sorted from low to high average depth of coverage) for 768 samples prepared from DNA of 384 plasmids prepared by RCA (blue diamonds) or miniprep (MP; green squares). The horizontal line that meets the y-axis indicates the 15× coverage threshold. MAD is the median absolute deviation.

Although the average depth of coverage for the 768 samples spanned over three orders of magnitude and displayed wide statistical variation (FIG. 3A), only 4% of the samples had an average coverage below 15×, an empirically determined point below which the sequence data is generally unreliable. Since the total yield of data in a MiSeq run is divided between the samples in the pool, it is most significant that the plasmid DNA samples had about twice the coverage variation compared to the RCA DNA samples. This implies that a greater percentage of samples will have reliable data if the pool contains only RCA DNA samples instead of plasmid DNA samples. The sequence data for each DNA assembly was identical whether prepared by RCA or plasmid miniprep, with three exceptions where the samples prepared from plasmid DNA apparently lost the insert, perhaps because cells containing empty plasmid swept the population. It was concluded that although both amplification methods can be used, plasmid DNA prepared by RCA is superior (e.g., in terms of generating less coverage variation) to that prepared by alkaline lysis for highly multiplexed plasmid sequencing on the MiSeq.

FIG. 9 illustrates how accurately RCA DNA can be transferred by Echo acoustic liquid system. During experiments, it was found that solutions of phage λ DNA at concentrations over about 20 ng/μL was not transferred by the Echo, apparently because long polymers can prevent ejection of emerging droplets. Since RCA DNA, like phage λ DNA, has a high molecular weight (≥50 kb), it was investigated how accurately RCA DNA was transferred by the Echo. A 384-well source plate was filled with precise concentrations of DNA generated from pure plasmid DNA using an Illustra Templiphi kit. More specifically, a source plate containing precise concentrations of DNA prepared by RCA of a single plasmid construct (actual ng/μL) was used to transfer one μL to the same wells of a low volume black assay plate (Costar 3677) on the Echo. The amount of transferred DNA was then assayed by Picogreen fluorescence. For each data point N=48 and the error bars are standard deviation. As shown in FIG. 9, the Echo accurately (>90%) and reliably transferred this DNA at concentrations up to 10 ng/μL.

### Example 5: Normalizing DNA Concentration Before Tagmentation not Necessary for RCA Prepared DNA

Since tagmentation reaction involves combining the DNA template with the Tn5 enzyme at a relatively precise protein to DNA ratio, the Echo acoustic liquid transfer system was considered for diluting the RCA preps to 2.5 ng/μl. However, since normalizing DNA concentration for each sample individually for many samples is time and labor intensive, other options were explored for this step. After quantifying RCA DNA using PicoGreen, the BiomekFX robot was used to normalize DNA. This normalization process took about an hour for 4 plates. The normalized DNA was then used on the Echo to set up our tagmentation reactions. In parallel, one of the four plates was taken, and the DNA was uniformly diluted to the same volume (e.g., 5 μL of DNA to 35 μL water) across all samples on the plate. This method was chosen because the DNA generated by RCA tends to be relatively constant in concentration, more so than DNA prepared by minipreps. From the calculations of how much DNA was to be added to water using the BiomekFX robot, the ratio of 5 μL DNA to 35 μL water was the average dilution required for that plate in some implementations. FIG. 3B illustrates that the DNA size ranges for both treatments are similar. This result indicates that the size distributions of RCA DNA that had been normalized before tagmentation were very similar to those that had not been normalized. This suggests that DNA amplified by RCA is of even concentration across many samples. Therefore, to save time, this non-normalized plate can be used on the Echo to set up the tagmentation reactions.

### Example 6: Increasing the Number of Samples Receiving Sufficient Sequence Data

For a robust QC process, the samples should receive similar average read coverage and few should have less than 15× coverage. To achieve this, each sample in the pool should have a similar molar concentration of sequenceable fragments such that each forms a similar number of clusters on the MiSeq flow cell. When the same pool of Nextera libraries derived from the same set of plasmid constructs was sequenced in separate MiSeq runs, coverage was highly correlated between the runs (FIG. 10), indicating that coverage variation arises during preparation and pooling of the libraries, not during the Illumina sequencing process. The sequence of each sample obtained from the two runs was identical, verifying the reliability of the sequence data itself (data not shown).

The large deviation in average coverage across the sample population in FIG. 3 was observed early in the development of this method. Subsequently, the protocol was optimized, as described below, and the number of samples sequenced per run was steadily increased. To pool according to molar concentration, the average fragment size of thousands of samples must be determined in a reliable manner, which is time-consuming and labor-intensive. Therefore, here, the ways to minimize the variation in average fragment size across the libraries were explored so that pooling could be based on mass concentration. The effect of input DNA concentration on coverage variability was studied using a plate of precise concentrations of RCA DNA to generate Nextera libraries. This revealed that input DNA concentrations of 3-10 ng/μL gave relatively consistent coverage, whereas coverage variation, and coverage itself, increased significantly as input DNA concentration fell below 2.5 ng/μL. See FIG. 4. Thus, coverage variation could be reduced by using RCA DNA at 3-10 ng/μL for tagmentation. In addition, the workflow could be streamlined, because all samples could be diluted by a standard factor, instead of diluting each sample individually.

Samples at the edges of a plate sometimes had low concentrations, which were thought to be due to droplets veering to the sides such that reagents were not completely mixed at the bottom of wells. To mitigate this, plates were centrifuged at 1,000 g immediately after dispensing on the Echo in some implementations. Also, the entire volume of any sample with a low concentration was decided to be added to the pool, because such samples then had a chance of receiving coverage without significantly affecting the coverage of other samples.

The protocol changes discussed above were implemented for the parallel sequencing of 4078 plasmids. FIG. 5 shows that the coverage variation and statistics for this MiSeq run were significantly improved over the run shown in FIG. 3A, with 98.4% receiving over 15× average coverage. Of the 1.6% samples with low coverage, most were found to be empty wells that had failed at the RCA step and would fail any QC method. Without wishing to be bound any theory, it was hypothesized that the slightly higher ratio of DNA to transposome during tagmentation reduced variation because the subsequent PCR to append the barcode adapter sequences uses a 30 second extension time that will not amplify fragments too large to form clusters. In other words, the higher DNA to protein ratio during tagmentation and the short PCR extension time may act to hold the variation within limits.

In the above QC of 4078 plasmids, the consumables cost was $2.68 at present value per MiSeq sample, which breaks down as shown in Table 3.

TABLE 3
Consumables costs at present value per sample when 4000 samples
are sequenced in parallel

| Item | Cost per sample |
|---|---|
| RCA reagent | $0.53 |
| Tagmentation | $0.90 |
| reagent | |
| PCR reagents | $0.36 |
| SPRI beads | $0.01 |
| MiSeq run kit | $0.23 |
| PicoGreen | $0.14 |
| Plates & tips | $0.51 |
| TOTAL | $2.68 |

Although this is almost $11 per assembly at present day value (because four replicates of each are sequenced), achieving only 1× coverage by Sanger sequencing of this same set of DNA assemblies would be about 10-fold more expensive and would include the need to order and track many primers to distribute the reads across the assemblies appropriately.

### Example 7: Analyzing the NGS QC Data

Aligning reads to a digital reference and choosing the best replicate of an assembly is conceptually simple, but requires rapid, parallel analysis of many datasets. The SAMTOOLS and BCFTOOLS (Ramirez-Gonzalesz et al. (2012) Source Code Biol. Med. 7:6) were initially tested to identify single-nucleotide polymorphism (SNPs) and indels, but it was difficult to find appropriate settings to reliably call all mutations found in the plasmids. A possible cause for this could be the high read coverage seen in some samples (approaching 1000×), which may hinder some part of the mutation calling algorithm. Subsampling the sequencing data in these cases would not be ideal as this reduces resolution of SNP frequency and complicates base calling in regions of low coverage. Another possible cause is that the DNA samples may be mixed populations that do not resemble the diploid genomic samples against which these algorithms and tool sets were developed. For example, a SNP at 10% frequency does not match a heterozygous or homozygous situation. Interestingly, it was found that the features were identified correctly at the level of read alignment but sometimes missed by the calling algorithms.

Given the small size of the plasmids that were sequencing (compared to genomes), in certain embodiments of the present invention, a simple feature detection method was implemented based on the pileup file. Software was written in F# (fsharp.org) to call mutations and assign severity scores to features (e.g., SNPs and indels) based on their sequence context (e.g., part type and the probability that they could impair function). The software ranks the replicates of each assembly based on the number of mutations and their severity and reports which replicate best matches the digital template. In addition, the software stores all sequence variants found, along with other relevant information, in a postgreSQL database.

Finally, the software generates a graphic for each sample (FIG. 6) showing coverage and variant calls, which facilitates the investigation of specific cases when the algorithmic decision is in question. In FIG. 6, the top two show samples with differences between the reads and the reference, while the bottom two show samples that match the reference perfectly (not counting the vector). The green region (an area underneath jagged lines) shows the depth of coverage. Red and blue vertical bars along the x-axis indicate a SNP in the forward and reverse reads. Purple and yellow vertical bars along the x-axis indicate an indel in the forward and reverse reads. Note that even with less than 15× average coverage (bottom right), it is sometimes possible to obtain reliable QC data. At the bottom of each plot are the DNA parts in green (e.g., blank horizontal bars along the x-axis—R39309, R40174, R2663, R40200, R2663, R29189, R20770, R39300, and R2662) and the vector portions in yellow (e.g., hatched horizontal bars along the x-axis—V25745R and V25745L). The uneven coverage in these examples is mostly due to Poisson sampling during the sequencing process. Some of the uneven coverage might also be due to bias for or against certain sequence motifs by either the transposome (Ason (2004) J. Mol. Biol. 335: 1213-1225) or the polymerase used for the PCR (Aird et al. (2011) Genome Biol. 12: R18). On the other hand, it might also be an indication of sequence discrepancies that should be more closely investigated.

In the run with 4078 samples described herein, 4056 were four replicates of 1014 constructs assembled by yeast homologous recombination. The remaining 22 samples were internal process controls, which were not used for data analysis. Table 4 shows the statistics for the sequence differences between the samples and the digital reference sequences.

TABLE 4
Sequence difference statistics for the four replicates of 1014
assemblies assembled by yeast homologous recombination.

| | Percent of 4056 |
|---|---|
| | samples or 1014 |
| Statistic | constructs |
| Samples exactly matching the reference | 54% |
| Samples with only one SNP or one indel | 23% |
| Samples with more than one SNP or indel | 16% |
| Samples misassembled (zero coverage for >200bp) | 5.8% |
| Constructs having at least one replicate matching | 73% |
| reference | |
| Constructs having at least one replicate correctly | 99% |
| assembled | |

The importance of replicates is highlighted by the fact that although 5.8% of the samples were misassembled, only 1% of the constructs had no correctly assembled replicate.

When a SNP or indel is present in only one replicate of a construct, this is likely due to errors in the primers or errors by the polymerase during PCR amplification of parts. Alternatively, errors may arise during RCA for MiSeq sample preparation. The frequency of this type of mutation appears consistent with the known fidelity of the polymerases (McInerney et al. (2014) Mol. Biol. Int. 2014: 287430), or with the reported frequency of errors in oligonucleotide primers (Hecker and Rill (1998) Biotechniques 24: 256-260). Many indels were located at homopolymers, which are known to be susceptible to contraction during replication and are also prone to sequencing artefacts even on the Illumina platform. When the same SNPs or indels are present in all four replicates, or in the same part in different constructs, they are most likely due to errors in either the digital reference sequence (i.e. data entry) or the template used for PCR amplification of the part. Several errors were due to the use of a physical part for the PCR template that was not the same as the part specified in the digital request. The frequency of this type of mutation was higher than anticipated, which can be further reduced. Since the run with 4078 samples described here, this NGS QC process has been used in more than ten assembly cycles, thus accumulating a large amount of NGS QC data. A comprehensive analysis of this data can be used to identify how the assembly process generates the different types of mutations, which can illustrate areas of improvement for the DNA assemblies.

### Exemplary Protocol for Preparing Plasmids for Sequencing Quality Control

All liquid transfers are accomplished using automation. All transfers less than 2 μL were accomplished using the Echo and all transfers greater than 2 μL were accomplished using a BiomekFX or NX.
1) Pick E. coli colonies into LB and grow overnight to saturation
2) Prepare DNA using a rolling circle amplification assay (methods here reflect the protocol from the GE kit)
   a. Dilute culture 1:15
      i. Add 23 mL of water to a 384-well PCR plate (BioRad)
      ii. Add 2 mL of culture to the water
   b. Seal plates very well, boil 3 minutes at 95° C., then hold at 10° C.
   c. Add 2 mL of denature buffer to a new 384-well PCR plate (BioRad)
   d. Add 2 mL of boiled culture to denature buffer
   e. Add 4 mL of reaction buffer to culture
   f. Incubate at 30° C. overnight
3) Quantify DNA concentration using PicoGreen assay
   a. Dilute RCA 1:12 (but the dilution should verify with Picogreen assay and DNA concentration needs to be adjusted, if needed)
      i. add 16 mL water to 8 mL of RCA reaction
      ii. add 30 mL water to Echo qualified source plate
      iii. add 10 mL of diluted RCA reaction to Echo plate
   b. Mix PicoGreen buffer according to following recipe:
   c. Add 1 mL of diluted RCA product or DNA standard to low volume black plates (Costar)
   d. Add 19 mL buffer to low volume black plates (Costar)
   e. Read fluorescence on an M5 reader with Picogreen protocol, 384-well plate, medium sensitivity.
   f. Record DNA concentration
4) Tagmentation of DNA samples in 0.5 μL reactions
   a. Add 200 nL of DNA to a 384-well PCR plate (BioRad)
   b. Premix enzyme into tagmentation buffer.
      i. Each reaction will receive 250 nL of buffer and 50 nL of tagmentation enzyme. Be sure to make enough to account for dead volume and pipetting error
   c. Add 300 nL of premix to DNA
   d. Incubate 10 min at 55° C.
5) Remove protein from DNA
   a. Add 125 nL (0.25 tagmentation volumes) of 0.5% SDS in TE to tagmented DNA, mix gently
   b. Incubate at room temperature (25-27° C.) for 5 min
6) Add unique primer barcode combinations to each sample
   a. Generate a worklist for the liquid handler to add 125 nL each of i5 forward and i7 reverse primers (100 mM). Ensure each combination is unique and is recorded for the MiSeq sequencer.
7) Prepare and Perform PCR reaction
   a. Prepare master mix including enough for dead volume and pipetting error, according to this table:

PCR Composition (per well)

| mL | Reagent |
|---|---|
| 20.275 | Water |
| 2.5 | 10x Thermopol buffer |
| 0.5 | MgSO₄ |
| 0.5 | dNTPS |
| 0.05 | i5 terminal primer (100 mM) |
| 0.05 | i7 terminal primer (100 mM) |
| 0.25 | Vent polymerase |
| 0.875 | DNA + SDS + primers |
| 25 | Total |

b. Add 24.325 μL master mix to each PCR tube, mixing gently
c. Cycle as follows:
   i. 72° C. 3 minutes, [98° C. for 10 seconds, 63° C. for 30 seconds, 72° C. for 30 seconds]×12 cycles, hold at 10° C.
8) Clean up PCR reactions
   a. Mix PCR reactions with 0.6 volumes of SPRI beads (i.e. 15 μL of slurry to 25 μL of PCR)
   b. Follow the manufacturer's protocol, including washing twice with 70% ethanol, drying the beads, eluting with 30 μL TE, and transferring 27 μL to destination plate
9) Quantify DNA concentration using PicoGreen assay
   a. Make 1/300 dilution of Picogreen reagent in TE+0.05% Tween20. Make 7.5 mL per plate to be assayed, plus 5 mL for step 11 below.
   b. Add 15 μL buffer to black plate (save about 5 mL for step 11 below)
   c. Add 5 μL sample to each plate, 5 μL ladder to appropriate wells, mix
   d. Read fluorescence on M5 reader with Picogreen protocol, 384-well plate, medium sensitivity.
   e. Analyze DNA concentrations
10) Pool samples
   a. Determine volume of each sample to add to pool, assuming average fragment size of 500 base pairs and normalizing for plasmid length
   b. A lower limit of 2 μL and an upper limit of 25 μL are used for volume transfers
11) Concentrate and quantify sample pool
   a. Add 500 μL to each of 2 Microcon spin filters and centrifuge 10 minutes at 1000 g
   b. Mix 75 μL buffer+5 μL sample, determine concentration
12) Characterize size of pool.
   a. Measure the distribution of fragment sizes using a Bioanalyzer, Fragment Analyzer, or by integrating the signal intensity along an agarose gel.
   b. Calculate concentration (nM) using PicoGreen value and size
      i. nM=ng/μL×1,000,000/(660×avg size)
13) Load MiSeq
   a. Dilute pool to 1.1 nM with water
   b. Denature: mix 18 μL of pool+2 μL of 1M NaOH, incubate RT 5 minutes
   c. Add 980 μL ice cold HT buffer, mix
   d. Neutralize: add 2 μL 1M HCl to size of tube, mix thoroughly and immediately
   e. Dilute pool to 12 pM with HT buffer, mix
   f. Load 600 μL into MiSeq cartridge
   g. Follow manufacturer's instructions

It should be appreciated that the specific steps illustrated in the exemplary protocol provides a particular method of preparing plasmids. Other sequences of steps may be performed according to alternative embodiments. For example, alternative embodiments of the present invention may perform the steps outlined above as multiple sub-steps as appropriate to the individual step. Furthermore, additional steps may be added or removed depending on the particular applications. For example, step 9) of quantifying DNA concentration using PicoGreen assay can be omitted. In another example, the DNA samples can be pooled without normalizing the concentration in step 10).

One or more features from any embodiment described herein may be combined with one or more features of any other embodiment without departing from the scope of the invention.

All publications, patents and patent applications cited in this specification are incorporated herein by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of preparing a plurality of polynucleotides for simultaneous sequencing, the method comprising:
for each input polynucleotide of a plurality of input polynucleotides,
(a) amplifying the input polynucleotide by rolling circle amplification (RCA) in an RCA solution to generate a target polynucleotide;
(b) diluting the RCA solution comprising the target polynucleotide by a standard dilution factor;
(c) generating a reaction mixture having a volume of about 0.005 μL to about 2 μL and comprising tagged polynucleotide fragments by contacting the diluted RCA solution comprising the target polynucleotide with transposases pre-loaded with transposon end sequences to fragment and tag the target polynucleotide;
(d) removing the transposases from the tagged polynucleotide fragments, thereby generating a reaction solution; and
(e) performing a polymerase chain reaction (PCR) with the reaction solution comprising the tagged polynucleotide fragments, wherein the PCR utilizes adapter primers comprising barcode sequences that are capable of hybridizing to the tagged polynucleotide fragments to generate barcoded polynucleotide fragments.

2. The method of claim 1, further comprising:
(f) combining the barcoded polynucleotide fragments generated for each input polynucleotide of the plurality of input polynucleotides;
(g) sequencing the combined barcoded polynucleotide fragments in step (f) in a single sequencing run to generate sequence reads;
(h) sorting the sequence reads from the sequencing run using the barcode sequences associated with the each input polynucleotide; and
(i) aligning and assembling the sequence reads for the each input polynucleotide to generate a consensus sequence of the input polynucleotide.

3. The method of claim 1, wherein the barcode sequences are selected from the group consisting of SEQ ID NO: 1 through SEQ ID NO: 192.

4. The method of claim 1, wherein the plurality of input polynucleotides is at least 1000.

5. (canceled)

6. The method of claim 4, wherein the input polynucleotide is a plasmid DNA.

7. The method of claim 6, wherein the plasmid DNA comprises a DNA assembly of a plurality of DNA components.

8. The method of claim 6, wherein the input polynucleotide is a plasmid and the combined barcoded polynucleotide fragments are generated from at least 1000 plasmids.

9. (canceled)

10. The method of claim 8, wherein less than 2 percent of the plasmids have less than 15 times average sequencing coverage.

11. The method of claim 10, wherein the reaction mixture has a volume of about 0.5 μL.

12. The method of claim 1, wherein the standard dilution factor is determined by:
(a) measuring a concentration of the target polynucleotide in the RCA solution for at least a portion of the plurality of input polynucleotides;
(b) determining an average concentration of the target polynucleotide in the RCA solution for the at least the portion of the plurality of input polynucleotides;
(c) calculating the standard dilution factor by dividing the average concentration by 5 ng/μL.

13. The method of claim 1, wherein the diluted RCA solution comprises the target polynucleotide at a concentration between about 3 ng/μL and about 10 ng/μL.

14. The method of claim 1, wherein the transposases are removed from the tagged polynucleotide fragments by treating the reaction mixture from step (c) under a dissociation condition.

15. (canceled)

16. The method of claim 14, wherein the dissociation condition comprises adding a dissociation solution comprising sodium dodecyl sulfate (SDS).

17-20. (canceled)

21. The method of claim 1, further comprising, after the PCR,
(f) removing small polynucleotide fragments from PCR products;
(g) quantifying a concentration of the barcoded polynucleotide fragments from step (f) for each input polynucleotide; and
(h) determining a volume of the barcoded polynucleotide fragments in step (f) to add to a pool assuming an average polynucleotide fragment size of 500 base pairs and normalizing for a length of the input polynucleotide.

22. The method of claim 21, further comprising filtering the combined barcoded polynucleotide fragments to remove small fragments having a size less than about 300 base pairs.

23. A method of preparing a plurality of polynucleotides for sequencing, the method comprising:
(a) generating a reaction mixture having a volume of about 0.005 μL to about 2 μL and comprising tagged polynucleotide fragments by contacting a target polynucleotide with transposases pre-loaded with transposon end sequences to fragment and tag the target polynucleotide; and
(b) performing a polymerase chain reaction (PCR) with a reaction solution comprising the reaction mixture comprising the tagged polynucleotide fragments, wherein the PCR utilizes adapter primers comprising barcode sequences capable of hybridizing to the tagged polynucleotide fragments to generate barcoded polynucleotide fragments.

24. The method of claim 23, further comprising:
(c) repeating steps (a) and (b) of claim 23 to generate barcoded polynucleotide fragments from a plurality of target polynucleotides, wherein the barcoded polynucleotide fragments from each of the plurality of target polynucleotides comprise a unique barcode sequence;
(d) combining the barcoded polynucleotide fragments generated from the plurality of target polynucleotides; and
(e) sequencing the combined barcoded polynucleotide fragments in step (d) in a single sequencing run to generate sequence reads.

25. The method of claim 23, further comprising diluting the reaction solution in step (b) by at least 10-fold with an aqueous solution prior to performing the PCR.

26. (canceled)

27. The method of claim 23, wherein the target polynucleotide is provided by rolling amplification of a plasmid DNA.

28. The method of claim 23, wherein the combined barcoded polynucleotide fragments are generated from at least 1000 plasmid DNA.

29. (canceled)

30. The method of claim 23, wherein the barcode sequences are selected from the group consisting of SEQ ID NO: 1 through SEQ ID NO: 192.

31. The method of claim 23, wherein the reaction mixture has a volume of about 0.5 μL.

32. (canceled)

33. A method of preparing a plurality of polynucleotides for sequencing, the method comprising:
for each input polynucleotide of a plurality of input polynucleotides,
(a) amplifying the input polynucleotide by rolling circle amplification (RCA) in an RCA solution to generate a target polynucleotide;
(b) diluting the RCA solution comprising the target polynucleotide by a standard dilution factor;
(c) generating a reaction mixture having a volume of about 0.005 μL to about 2 μL and comprising tagged polynucleotide fragments by contacting the diluted RCA solution comprising the target polynucleotide with transposases pre-loaded with transposon end sequences to fragment and tag the target polynucleotide;
(d) adding a dissociation solution to the reaction mixture to remove the transposases from the tagged polynucleotide fragments, thereby generating a reaction solution;
(e) diluting the reaction solution with an aqueous solution;
(f) adding to the diluted reaction solution a pair of adapter primers comprising barcode sequences capable of hybridizing to the tagged polynucleotide fragments;
(g) performing a polymerase chain reaction (PCR) with the diluted reaction solution of step (f) and terminal primers to generate barcoded polynucleotide fragments, wherein the terminal primers are capable of hybridizing to the barcoded polynucleotide fragments;
(h) combining the barcoded polynucleotide fragments generated in step (g) for each input polynucleotide of the plurality of input polynucleotides;
(i) sequencing the combined barcoded polynucleotide fragments of step (h) in a single sequencing run to generate sequence reads;
(j) sorting the sequence reads from the sequencing using the barcode sequences associated with each input polynucleotide to assign each of the sequence reads to each input polynucleotide; and
(k) aligning and assembling the sorted sequence reads for each of the input polynucleotide to generate a consensus sequence of each input polynucleotide.

34.-41. (canceled)
